(19) United States
(12) Patent Application Publication
(10) Pub. No.: US 2024/0157070 A1
(43) Pub. Date: May 16, 2024

(54) DISPLAY UNIT FOR A BREATHING ASSISTANCE APPARATUS

(71) Applicant: Fisher & Paykel Healthcare Limited, Auckland (NZ)

(72) Inventors: Sean Joel BABBAGE, Auckland (NZ); Andrew Paul Maxwell SALMON, Auckland (NZ); Kevin Peter O'DONNELL, Auckland (NZ); Jonathan James Eric MACE, Auckland (NZ); Michael James NIHOTTE, Auckland (NZ); Kallista Renée PIJNAKER, Auckland (NZ); Paul Fleming BUCKLEY, Auckland (NZ); Peter Alan SEEKUP, Auckland (NZ); Lujia CHEN, Auckland (NZ); Ryan James MARTIN, Auckland (NZ)

(21) Appl. No.: 18/259,164

(22) PCT Filed: Dec. 23, 2021
(86) PCT No.: PCT/IB2021/062281

§ 371 Date: Jun. 23, 2023

Related U.S. Application Data
(60) Provisional application No. 63/131,192, filed on Dec. 28, 2020.

(51) Int. Cl. A61M 16/00; A61M 16/08; A61M 16/16; G06F 3/14; H05K 5/00

(52) U.S. Cl. CPC A61M 16/0051; A61M 16/0816; A61M 16/16; H05K 5/0086; A61M 2205/3561; A61M 2205/502; A61M 2205/583; G06F 3/14

(57) ABSTRACT

A portable display unit 3000, 3500, 4000 for a breathing assistance apparatus, the portable display unit comprising: a display unit housing 3001, 3501, 4000; a screen 3051, 3551, 4051; and a tethering component for tethering the portable display unit 3000, 3500, 4000 with abase unit 50 of the breathing assistance apparatus.

### TECHNICAL FIELD

The present disclosure relates to a display unit for a breathing assistance apparatus. The present disclosure also relates to a breathing assistance apparatus.

### BACKGROUND ART

Breathing assistance apparatuses are used in various environments such as hospital, medical facility, residential care, retirement facilities, hospices or home environments to deliver a flow of gas to users or patients. The breathing assistance apparatuses come in various forms, such as a standalone humidifier apparatus, a continuous positive airway pressure (CPAP) apparatus, a high flow apparatus, or a ventilator.

The breathing assistance apparatuses typically have one or more accessories such as a breathing conduit and a patient interface such as a cannula or mask for delivering gases to a patient. The conduit enables gases to be delivered from the housing of the breathing assistance apparatus to the patient. For example, the apparatus may be placed on a floor or other support surface, and the patient may be in a bed. The breathing assistance apparatus may have a recess for receipt of a humidification chamber. The humidification chamber will receive liquid from, for example, a flexible liquid bag that delivers liquid to a humidifier humidification chamber via one more tubes. Alternatively, the humidification chamber can be removed and refilled as required. The recess will contain a heater plate to heat the humidification chamber, to humidify gases passing through the humidification chamber. The humidified gases are then delivered to the patient.

The breathing assistance apparatus may have a display, to display information relating to the use of the apparatus and/or to enable a user to control functioning of the apparatus.

### SUMMARY

In a first aspect of the disclosure, in accordance with certain features, aspects and advantages of at least one of the embodiments disclosed herein, a portable display unit for a breathing assistance apparatus is disclosed, the portable display unit comprising: a display unit housing; a screen; and a tethering component for tethering the portable display unit with a base unit of the breathing assistance apparatus.

In some configurations, the tethering component is configured for physically tethering the portable display unit with the base unit of the breathing assistance apparatus.

In some configurations, the tethering component comprises a cable.

In some configurations, the cable is resiliently flexible and/or is retractable.

In some configurations, the cable is for communicating power to the portable display unit from the base unit and/or for communicating data between the portable display unit and the base unit.

In some configurations, the portable display unit further comprises at least one wire for communicating power to the portable display unit from the base unit and/or for communicating data between the portable display unit and the base unit

In some configurations, the tethering component comprises a component of a wireless communication interface for communicating data between the portable display unit and the base unit.

In some configurations, the portable display unit further comprises at least one connection feature for removably connecting the portable display unit to the base unit.

In a further aspect of the disclosure, in accordance with certain features, aspects and advantages of at least one of the embodiments disclosed herein, a portable display unit for a breathing assistance apparatus is disclosed, the portable display unit comprising: a display unit housing; a screen; and a cable for physically tethering the portable display unit with a base unit of the breathing assistance apparatus.

In some configurations, the cable is resiliently flexible and/or is retractable.

In some configurations, the cable is for communicating power to the portable display unit from the base unit and/or for communicating data between the portable display unit and the base unit.

In some configurations, the portable display unit comprises at least one wire for communicating power to the portable display unit from the base unit and/or for communicating data between the portable display unit and the base unit.

In some configurations, the portable display unit comprises a wireless communication interface for communicating data between the portable display unit and the base unit.

In some configurations, the portable display unit further comprises at least one connection feature for removably connecting the portable display unit to the base unit.

In a further aspect of the disclosure, in accordance with certain features, aspects and advantages of at least one of the embodiments disclosed herein, a portable display unit for a breathing assistance apparatus is disclosed, the portable display unit comprising: a display unit housing; a screen; a communication interface for communicating data between the base unit and the portable display unit; and at least one connection feature for removably connecting the portable display unit to the base unit.

In some configurations, the at least one connection feature is arranged to removably connect the portable display unit to a housing of the base unit.

In some configurations, the at least one connection feature is arranged to removably connect the portable display unit to the housing of the base unit by a sliding action of the portable display unit relative to the base unit.

In some configurations, the portable display unit comprises at least one support feature for supporting the portable display unit on a support surface.

In some configurations, at least one support feature and at least one connection feature are provided by the same structural element.

In some configurations, the at least one support feature is arranged to support the portable display unit on a substantially horizontal support surface or on a medical pole.

In some configurations, the at least one support feature is arranged to enable the angle of the screen to be adjusted relative to the support surface.

In some configurations, an angular orientation of the screen can be adjusted relative to the display unit housing or wherein an angular orientation of the screen can be adjusted relative to the at least one connection feature.

In some configurations, the communication interface comprises at least one wire or wire connector for communicating data and/or control signals between the portable display unit and the base unit.

In some configurations, the communication interface comprises a wireless communication interface for communicating data and/or control signals between the portable display unit and the base unit.

In a further aspect of the disclosure, in accordance with certain features, aspects and advantages of at least one of the embodiments disclosed herein, a portable display unit for a breathing assistance apparatus is disclosed, the portable display unit comprising: a display unit housing; a screen; a communication interface for communicating data between the base unit and the portable display unit; and at least one support feature for supporting the portable display unit on a support surface.

In some configurations, the at least one support feature is arranged to support the portable display unit on a substantially horizontal support surface or on a medical pole.

In some configurations, the at least one support feature is arranged to enable the angle of the screen to be adjusted relative to the support surface.

In some configurations, the communication interface comprises at least one wire for communicating data signals between the portable display unit and the base unit.

In some configurations, the communication interface comprises a wireless communication interface for communicating data between the portable display unit and the base unit.

In some configurations, the screen is a touch screen to enable the user to interact with the screen to control one or more functions of the breathing assistance apparatus.

In a further aspect of the disclosure, in accordance with certain features, aspects and advantages of at least one of the embodiments disclosed herein, the combination of a breathing assistance apparatus and a portable display unit is disclosed, the combination comprising: a breathing assistance apparatus comprising a base unit with a housing having a gas port; and the portable display unit as outlined in relation to any of the above embodiments or aspects.

In some configurations, the portable display unit is connectable to and separable from the base unit.

In some configurations, the portable display unit is tethered with the base unit.

In some configurations, the portable display unit is physically tethered with the base unit.

In some configurations, the portable display unit is wirelessly tethered with the base unit for communicating data between the portable display unit and the base unit.

In some configurations, the base unit comprises a first display fixed to the housing of the base unit, and wherein the portable display unit provides a second display for the breathing assistance apparatus.

In a further aspect of the disclosure, in accordance with certain features, aspects and advantages of at least one of the embodiments disclosed herein, a breathing assistance apparatus is disclosed, the breathing assistance apparatus comprising:
a humidifier;
a base unit;
and a portable display unit comprising:
   a display unit housing;
   a screen;
   a communication interface for communicating data between the base unit and the portable display unit;
   and at least one connection feature for removably connecting the portable display unit to the base unit.

In some configurations, the humidifier is provided in the base unit.

In some configurations, the communication interface comprises at least one wire for communicating data and/or control signals between the portable display unit and the base unit.

In some configurations, the communication interface comprises a wireless communication interface for communicating data and/or control signals between the portable display unit and the base unit.

In a further aspect of the disclosure, in accordance with certain features, aspects and advantages of at least one of the embodiments disclosed herein, a breathing assistance apparatus is disclosed, the breathing assistance apparatus comprising:
a base unit;
a heating element positioned on or within the base unit;
a humidification chamber associated with the heating element, such that the heating element is in thermal communication with the humidification chamber;
a controller in electronic communication with the heating element; and
a display unit, wherein the display unit is separable from the base unit, such that the display unit can be separated from the base unit and reconnected to the base unit,
wherein the controller is in electronic communication with the display unit when the display unit is connected to and separated from the base unit, such that data and/or power signals can be transmitted from the controller to the display unit and data can be transmitted from the display unit to the controller.

In some configurations, the breathing assistance apparatus is configured to receive gases from an external source.

In some configurations, the breathing assistance apparatus comprises a flow generator in the base unit to generate a flow of pressurised gases.

In some configurations, the humidification chamber is removable from the base unit.

In some configurations, the display unit is physically tethered to the base unit to enable transmission of power signals from the controller to the display unit.

In some configurations, the display unit is physically tethered to the base unit to enable transmission of data from the controller to the display unit and to enable transmission of data from the display unit to the controller.

In some configurations, the display unit is wirelessly tethered to the base unit to enable transmission of data from the controller to the display unit and to enable transmission of data from the display unit to the controller.

In a further aspect of the disclosure, in accordance with certain features, aspects and advantages of at least one of the embodiments disclosed herein, a breathing assistance apparatus is disclosed, the breathing assistance apparatus comprising:
a flow generator to generate a flow of pressurised gases;
a humidifier to humidify the gases; and
a display unit including a display unit housing, a screen, and a tethering component for tethering the portable display unit with at least one of the components of the breathing assistance apparatus,
wherein the components are modular such that at least one of the components is separable from at least one other of the components.

In some configurations, the display unit is separable from at least one other component of the breathing assistance apparatus.

In some configurations, the humidifier and flow generator are separable from each other.

In some configurations, the humidifier is in a humidifier housing, the flow generator is in a separate flow generator housing, and the humidifier housing can be removably coupled to the flow generator housing.

In some configurations, the display unit is separable and removably coupled to the flow generator housing.

In some configurations, the display unit is separable and removably coupled to the humidifier housing.

In some configurations, the humidifier comprises a first display unit, the flow generator comprises a second display unit, and both display units are removable and portable.

In some configurations, the humidifier and the flow generator are in a common housing.

In some configurations, the humidifier comprises a heater plate that is attached to the common housing and a humidification chamber that can be removably positioned within a portion of the common housing such that the humidification chamber is in thermal communication with the heater plate.

In some configurations, the display unit is separable and removably couplable to the common housing.

In a further aspect of the disclosure, in accordance with certain features, aspects and advantages of at least one of the embodiments disclosed herein, a portable display unit for a breathing assistance apparatus is disclosed, wherein the portable display unit can be positioned in an operative position that is separate from a base unit of the breathing assistance apparatus and vertically higher than the base unit.

In a further aspect of the disclosure, in accordance with certain features, aspects and advantages of at least one of the embodiments disclosed herein, a breathing assistance apparatus is disclosed, the breathing assistance apparatus comprising: a base unit; and a portable display unit, wherein the portable display unit can be positioned in an operative position that is separate from the base unit and vertically higher than the base unit.

In some configurations, the breathing assistance apparatus comprises a humidifier.

In some configurations, the humidifier can be positioned below patient head height while the portable display unit can be positioned in the operative position so as to be readily viewable/usable.

In some configurations, the breathing assistance apparatus further comprises a breathing circuit in fluid communication with the humidifier, the breathing circuit comprising a patient interface.

In some configurations, when the when the humidifier is placed below patient head height, any condensate which accumulates in the breathing circuit is encouraged to return to the humidifier rather than collecting at the patient interface.

In some configurations, the humidifier may be placed under patient hip height in order to let condensate drain into a humidification chamber of the humidifier.

In some configurations, the method comprises placing the portable display unit above the base unit and the humidifier.

In some configurations, when the portable display unit is above the base unit and/or the humidifier, the portable display unit enables user interaction and control of the breathing assistance apparatus from an advantageous position.

In a further aspect of the disclosure, in accordance with certain features, aspects and advantages of at least one of the embodiments disclosed herein, a method of using a portable display unit of a breathing assistance apparatus is disclosed, the breathing assistance apparatus comprising a base unit including a humidifier, the portable display unit being tethered to the base unit, the method comprising the steps of: disconnecting the portable display unit from the base unit, and positioning the portable display unit above the base unit such that the display unit is visible to a user.

In some configurations, the method comprises extending the tether or manipulating the tether to position the portable display unit in a location that is at eye level of the user or adjacent a patient.

In some configurations, the breathing assistance apparatus comprises a humidifier.

In some configurations, the method comprises positioning the humidifier below patient head height and positioning the portable display unit in the operative position so as to be readily viewable/usable.

In some configurations, the breathing assistance apparatus further comprises a breathing circuit in fluid communication with the humidifier, the breathing circuit comprising a patient interface, and wherein when the when the humidifier is placed below patient head height, any condensate which accumulates in the breathing circuit is encouraged to return to the humidifier rather than collecting at the patient interface.

In some configurations, the method comprises placing the humidifier under patient hip height in order to let condensate drain into a humidification chamber of the humidifier.

In some configurations, the method comprises placing the portable display unit above the base unit and/or the humidifier.

In some configurations, when the portable display unit is above the base unit and/or the humidifier, the portable display unit enables user interaction and control of the breathing assistance apparatus from an advantageous position.

In a further aspect of the disclosure, in accordance with certain features, aspects and advantages of at least one of the embodiments disclosed herein, a breathing assistance apparatus is provided, the breathing assistance apparatus comprising:
a base unit;
a portable display unit comprising:
   a display unit housing;
   a screen; and
   at least one connection feature for removably connecting the portable display unit to the base unit and to a different support component;
and a tethering component for tethering the portable display unit with the base unit.

In some configurations, the tethering component comprises a cable for physically tethering the portable display unit with the base unit.

In some configurations, the cable is for communicating power to the portable display unit from the base unit and/or for communicating data between the portable display unit and the base unit.

In some configurations, the tethering component comprises a component of a wireless communication interface for communicating data between the portable display unit and the base unit.

In some configurations, the breathing assistance apparatus further comprises a humidifier.

In some configurations, the humidifier is integral with the base unit.

In some configurations, the humidifier is configured to be placed below patient head height.

In some configurations, the humidifier is configured to be placed below a patient or below a patient bed level.

In some configurations, the breathing assistance apparatus further comprises a breathing circuit in fluid communication with the humidifier, the breathing circuit comprising a patient interface to deliver gases to a patient.

In some configurations, the breathing circuit and the humidifier are configured such that when the humidifier is placed below patient head height, any condensate which accumulates in the breathing circuit is encouraged to return to the humidifier rather than collecting at the patient interface.

In some configurations, the portable display unit is configured to be placed above the humidifier.

In some configurations, the portable display unit can be positioned in an operative position that is separate from the base unit and the humidifier and vertically higher than the base unit and the humidifier.

In some configurations, the portable display unit and the humidifier are configured such that when the portable display unit is above the humidifier, the portable display unit enables user interaction and control of the breathing assistance apparatus from an elevated position above the humidifier, optionally at or near eye level of a user.

In some configurations, the elevated position is separate from the base unit and vertically higher than the base unit and humidifier.

In some configurations, the at least one connection feature comprises a first connection component.

In some configurations, the first connection component is engageable with a second connection component located on the base unit and/or on the different support component.

In some configurations, the first connection component and the second connection component are components of a sliding connector.

In some configurations, the sliding connector comprises a dovetail connector.

In some configurations, the at least one connection feature further comprises a magnet.

In some configurations, the magnet is configured to cooperate with a magnet or a magnetisable material on the base unit and/or on the different support component.

In some configurations, the portable display unit comprises a retention feature that is configured to positively engage with a complementary retention feature on the base unit upon connection of the portable display unit with the base unit.

In some configurations, the at least one connection feature comprises a first connection feature for removably connecting the portable display unit to the base unit and comprises a second connection feature for removably connecting the portable display unit to the different support component, wherein the first connection feature is different to the second connection feature.

In some configurations, the first connection feature comprises a magnet.

In some configurations, the magnet is configured to cooperate with a magnet or a magnetisable material on the base unit.

In some configurations, the first connection feature comprises a magnetisable material that is configured to cooperate with a magnet on the base unit.

In some configurations, the at least one connection feature comprises a first component of a sliding connector located on the portable display unit.

In some configurations, the first component is engageable with a second component of the sliding connector located on the different support component.

In some configurations, the base unit has any one or more of the features outlined herein.

In a further aspect of the disclosure, in accordance with certain features, aspects and advantages of at least one of the embodiments disclosed herein, a breathing assistance apparatus is provided, the breathing assistance apparatus comprising:
a base unit;
a portable display unit comprising:
   a display unit housing; and
   a screen;
and a tethering component for tethering the portable display unit with the base unit;
wherein the base unit comprises at least one complementary connection feature for cooperating with at least one connection feature on the portable display unit for removably connecting the portable display unit to the base unit.

In some configurations, the tethering component comprises a cable for physically tethering the portable display unit with the base unit.

In some configurations, the cable is for communicating power to the portable display unit from the base unit and/or for communicating data between the portable display unit and the base unit.

In some configurations, the tethering component comprises a component of a wireless communication interface for communicating data between the portable display unit and the base unit.

In some configurations, the breathing assistance apparatus further comprises a humidifier.

In some configurations, the humidifier is integrated with the base unit.

In some configurations, the humidifier is configured to be placed below patient head height.

In some configurations, the humidifier is configured to be placed below a patient or below a patient bed level.

In some configurations, the breathing assistance apparatus further comprises a breathing circuit in fluid communication with the humidifier, the breathing circuit comprising a patient interface.

In some configurations, the breathing circuit and humidifier are configured such that when the humidifier is placed below patient head height, any condensate which accumulates in the breathing circuit is encouraged to return to the humidifier rather than collecting at the patient interface.

In some configurations, the display unit is configured to be placed above the humidifier.

In some configurations, the portable display unit can be positioned in an operative position that is separate from the base unit and the humidifier and vertically higher than the base unit and the humidifier.

In some configurations, the portable display unit and the humidifier are configured such that when the portable display unit is above the humidifier, the portable display unit allows for user interaction and control of the breathing assistance apparatus from an elevated position above the humidifier, optionally at or near eye level of a user.

In some configurations, the elevated position is separate from the base unit and vertically higher than the base unit and humidifier.

In some configurations, the at least one complementary connection feature comprises a second connection component of a connector that is engageable with a first connection component of the connector on the portable display unit.

In some configurations, the first connection component and the second connection component are components of a sliding connector.

In some configurations, the first component is engageable with the second component in a sliding action in a first direction of the first component relative to the second component, and wherein the first component and second component are not disengageable by attempted movement of the first component relative to the second component in a direction transverse to the first direction.

In some configurations, the at least one complementary connection feature comprises a magnet or a magnetisable material that is configured to cooperate with a magnet on the portable display unit.

In some configurations, the breathing assistance apparatus comprises at least one alignment feature on the base unit to assist with aligning the portable display unit with the base unit during connection of the portable display unit to the base unit.

In some configurations, the at least one alignment feature comprises an alignment surface that is configured to interact with a complementary alignment surface on the portable display unit.

In some configurations, the breathing assistance apparatus further comprises a display mounting component that comprises the complementary connection feature.

In some configurations, the display mounting component is removably connected to a main housing of the base unit.

In some configurations, the base unit comprises a complementary retention feature to positively engage with a retention feature of the portable display unit upon connection of the portable display unit with the base unit.

In some configurations, the complementary retention feature is selectively releasable from engagement with the retention feature of the portable display unit to enable disconnection of the portable display unit from the base unit.

In some configurations, the portable display unit has any one or more of the features outlined herein.

In a further aspect of the disclosure, in accordance with certain features, aspects and advantages of at least one of the embodiments disclosed herein, a portable display unit for a breathing assistance apparatus is provided, the portable display unit comprising: a display unit housing; a screen; at least one connection feature for removably connecting the portable display unit to a base unit of the breathing assistance apparatus; and a notification light.

In some configurations, the notification light is provided in or on at least one external surface of the display unit housing.

In some configurations, the notification light is visible from at least a portion of the portable display unit comprising the screen.

In some configurations, the display unit housing comprises a transparent portion in an upper surface of the display unit housing, and wherein the notification light is visible through the transparent portion.

In some configurations, the notification light projects from a rear of the display unit housing, such that the notification light is visible from at least a rear, an upper surface, a left side, a right side, and a base of the portable display unit.

In some configurations, the display unit housing comprises at least one transparent portion, and wherein the notification light is visible through the at least one transparent portion so as to be visible from 360 degrees around the display unit housing.

In some configurations, the notification light is configured to provide a visual alert in response to an alarm condition.

In some configurations, the alarm condition comprises an alarm condition of the portable display unit and/or an alarm condition of the breathing assistance apparatus.

In some configurations, the alarm condition of the portable display unit comprises one or more of: disconnection of a cable between the portable display unit and a base unit of the breathing assistance apparatus, physical or wireless communication disconnection from the breathing assistance apparatus, poor communications latency with the breathing assistance apparatus.

In some configurations, the portable display unit further comprises a notification speaker.

In some configurations, the notification speaker is configured to provide an audio alert in response to an alarm condition.

In some configurations, the alarm condition comprises an alarm condition of the portable display unit and/or an alarm condition of the breathing assistance apparatus.

In some configurations, the alarm condition comprises one or more of: disconnection of a cable between the portable display unit and a base unit of the breathing assistance apparatus, physical or wireless communication disconnection from the breathing assistance apparatus, poor communications latency with the breathing assistance apparatus.

In some configurations, the notification light and the notification speaker are configured to operate in combination to provide alerts.

In a further aspect of the disclosure, in accordance with certain features, aspects and advantages of at least one of the embodiments disclosed herein, the combination of a breathing assistance apparatus comprising: a base unit with a housing having a gas port; and the portable display unit as outlined above or herein is provided.

In some configurations, the base unit comprises a base unit notification light.

In some configurations, the base unit notification light is visible from at least a front of the base unit.

In some configurations, the base unit notification light is configured to provide a visual alert in response to an alarm condition.

In some configurations, the alarm condition comprises an alarm condition of the portable display unit and/or an alarm condition of the breathing assistance apparatus.

In some configurations, the alarm condition comprises one or more of: disconnection of a cable between the portable display unit and a base unit of the breathing assistance apparatus, physical or wireless communication disconnection from the breathing assistance apparatus, poor communications latency with the breathing assistance apparatus.

In some configurations, the portable display unit notification light and the base unit notification light are configured to operate in combination to provide alerts.

In some configurations, the base unit notification light is configured to indicate different colours in response to different notification conditions.

In some configurations, the base unit comprises an interconnect circuit board for providing data communication between the portable display unit and a main circuit board of the base unit, and wherein the base unit notification light is coupled to the interconnect circuit board.

In a further aspect of the disclosure, in accordance with certain features, aspects and advantages of at least one of the embodiments disclosed herein, a portable display unit for a breathing assistance apparatus is provided, the portable display unit comprising: a display unit housing; a screen; a cable for physically tethering the portable display unit to a base unit of the breathing assistance apparatus; a button; and a dial.

In some configurations, the cable is for communicating power to the portable display unit from the base unit and/or for communicating data between the portable display unit and the base unit.

In some configurations, the screen is a touch screen to enable the user to control one or more functions of the breathing assistance apparatus.

In some configurations, the button is a membrane button.

In some configurations, the dial comprises a rotatable component to provide a user input, and wherein the dial comprises a push button to provide a different user input.

In some configurations, the rotatable component surrounds a periphery of the push button, and wherein a pushing surface of the push button is recessed within the rotatable component.

In some configurations, the portable display unit further comprises a proximity sensor to determine user presence, wherein the screen is configured to dim or turn off if a user is not detected for a period of time.

In some configurations, the portable display unit further comprises an ambient light sensor, wherein the screen is configured to adjust in brightness in response to ambient light changes.

In some configurations, the portable display unit comprises at least one connection feature for removably connecting the portable display unit to the base unit and to a different support component.

In some configurations, the at least one connection feature comprises a first connection feature for removably connecting the portable display unit to the base unit and comprises a second connection feature for removably connecting the portable display unit to the different support component, wherein the first connection feature is different to the second connection feature.

In some configurations, an angular orientation of the display unit housing can be adjusted relative to the at least one connection feature and/or relative the base unit.

In a further aspect of the disclosure, in accordance with certain features, aspects and advantages of at least one of the embodiments disclosed herein, a portable display unit for a breathing assistance apparatus is provided, the portable display unit comprising: a display unit housing; a screen; a tethering component for tethering the portable display unit with a base unit of the breathing assistance apparatus; a button; and a dial.

In some configurations, the portable display unit has one or more of the features outlined above or herein.

In a further aspect of the disclosure, in accordance with certain features, aspects and advantages of at least one of the embodiments disclosed herein, a portable display unit for a breathing assistance apparatus is provided, the portable display unit comprising: a display unit housing defining an internal portion; a screen; and a cable for physically tethering the portable display unit to a base unit of the breathing assistance apparatus; wherein the portable display unit is hermetically sealed to prevent water and oxygen ingress into the internal portion of the display unit housing.

In some configurations, the cable is for communicating power to the portable display unit from the base unit and/or for communicating data between the portable display unit and the base unit.

In some configurations, the display unit housing comprises a first housing portion and a second housing portion, and wherein a seal or gasket is provided between the first housing portion and the second housing portion.

In some configurations, the seal or gasket comprises a compressible seal or compressible gasket, and wherein the compressible seal or compressible gasket is compressed between the first housing portion and the second housing portion in the assembled housing.

In some configurations, the portable display unit comprises electrical and/or electronic components housed within the internal portion of the display unit housing.

In some configurations, the electrical and/or electronics components comprises a circuit board.

In some configurations, the circuit board is surrounded by a sealing material to prevent water and oxygen ingress into the circuit board

In some configurations, the sealing material is further configured to provide shock absorption and/or heat dissipation for the circuit board.

In some configurations, the portable display unit further comprises a temperature sensor to determine if temperature in the internal portion of the portable display unit housing exceeds a threshold value.

In some configurations, the portable display unit further comprises an oxygen sensor to determine if oxygen is detected in the internal portion of the portable display unit housing above a threshold value.

In some configurations, the portable display unit comprises at least one connection feature for removably connecting the portable display unit to the base unit and to a different support component.

In some configurations, the at least one connection feature comprises a first connection feature for removably connecting the portable display unit to the base unit and comprises a second connection feature for removably connecting the portable display unit to the different support component, wherein the first connection feature is different to the second connection feature.

In a further aspect of the disclosure, in accordance with certain features, aspects and advantages of at least one of the embodiments disclosed herein, a portable display unit for a breathing assistance apparatus is provided, the portable display unit comprising: a display unit housing defining an internal portion; a screen; and a tethering component for tethering the portable display unit with a base unit of the breathing assistance apparatus; wherein the portable display unit is hermetically sealed to prevent water and oxygen ingress into the internal portion of the display unit housing.

In some configurations, the portable display unit has one or more of the features outlined above or herein.

In a further aspect of the disclosure, in accordance with certain features, aspects and advantages of at least one of the embodiments disclosed herein, a portable display unit for a breathing assistance apparatus is provided, the portable display unit comprising: a display unit housing having an upper surface; a screen; and a tethering component for tethering the portable display unit with a base unit of the breathing assistance apparatus; wherein the screen is recessed from the upper surface of the display unit housing.

In some configurations, at least a portion of the display unit housing comprises an impact absorbent material.

In some configurations, at least a portion of one or more corners and/or edges of the display unit housing comprise or are integrally formed from an impact absorbent material.

In some configurations, the impact absorbent material extends partly over the upper surface and a base of the display unit housing.

In some configurations, the impact absorbent material is integrally formed with the at least portion of the display unit housing.

In some configurations, the display unit housing defines an internal portion and the portable display unit further comprises electrical and/or electronic components housed within the internal portion of the display unit housing.

In some configurations, the electrical and/or electronics components comprises a circuit board.

In some configurations, the circuit board is surrounded by a material that is configured to provide shock absorption.

In some configurations, the portable display unit further comprises a sensor to detect if the portable display unit has been dropped or experiences an impact.

In some configurations, the sensor comprises an accelerometer or a gyroscope.

In some configurations, the portable display unit further comprises a notification light and/or notification speaker to provide a visual and/or audible alert if the sensor detects that the portable display unit has been dropped or experiences an impact.

In some configurations, the portable display unit comprises at least one connection feature for removably connecting the portable display unit to the base unit and to a different support component.

In some configurations, the at least one connection feature comprises a first connection feature for removably connecting the portable display unit to the base unit and comprises a second connection feature for removably connecting the portable display unit to the different support component, wherein the first connection feature is different to the second connection feature.

In a further aspect of the disclosure, in accordance with certain features, aspects and advantages of at least one of the embodiments disclosed herein, a breathing assistance apparatus is provided, the breathing assistance apparatus comprising:
a base unit;
a portable display unit comprising:
   a display unit housing; and
   a screen;
and a cable physically tethering the portable display unit with the base unit, wherein the cable is for communicating power to the portable display unit from the base unit and/or for communicating data between the portable display unit and the base unit, wherein the cable is connected to the portable display unit by an overmoulded portion of the cable which removably connects to a portion of the display unit housing and/or is connected to the base unit by a retention portion of the cable which removably connects to a portion of the base unit.

In some configurations, the cable is connected to the portable display unit by the overmoulded portion.

In some configurations, a shape of the overmoulded portion of the cable conforms to a shape of a slot in the portion of the display unit housing.

In some configurations, the shape of the overmoulded portion of the cable is such that the overmoulded portion of the cable can only be inserted into the slot in the portion of the display unit housing in a single orientation.

In some configurations, the overmoulded portion of the cable is configured to be fastened to the portion of the display unit housing by one or more fasteners to inhibit removal of the cable from the portable display unit.

In some configurations, the overmoulded portion of the cable provides a hermetic seal against water and oxygen ingress at the connection of the cable to the portable display unit.

In some configurations, the cable is connected to the base unit by the retention portion.

In some configurations, a shape of the retention portion of the cable conforms to a shape of a slot in the base unit.

In some configurations, the shape of the retention portion of the cable is such that the retention portion of the cable can only be inserted into the base unit in a single orientation.

In some configurations, the breathing assistance apparatus further comprises a sealing feature that provides a hermetic seal against water and oxygen ingress at the connection of the cable to the base unit.

In some configurations, the sealing feature is integral with the retention portion, is separate from the retention portion, or is both integral with the retention portion and separate from the retention portion.

In some configurations, the sealing feature comprises a grommet.

In some configurations, the cable has a length of up to about 2 m, optionally of between about 1.5 m and about 2 m.

In some configurations, the breathing assistance apparatus comprises at least one security feature to only allow connection of a genuine portable display unit to the base unit.

In some configurations, the at least one security feature is provided at least partly by the connection of the cable to the portable display unit and/or at the connection of the cable to the base unit.

In some configurations, the at least one security feature comprises a hardware and/or software module of the base unit to determine whether the connected portable display unit is genuine.

In some configurations, the hardware and/or software module is provided by a security circuit.

In some configurations, the security circuit comprises one or more encryption modules which allow an encrypted connection to be established through the cable if the portable display unit is genuine.

In some configurations, the cable is retractable.

In some configurations of any of the portable display units outlined in any of the above aspects, the display unit housing is hermetically sealed to prevent water and oxygen ingress into the internal portion of the display unit housing.

In some configurations, the display unit housing comprises a first housing portion and a second housing portion, and wherein a seal or gasket is provided between the first housing portion and the second housing portion.

In some configurations, the portable display unit comprises electrical and/or electronic components housed within the internal portion of the display unit housing.

In some configurations, the electrical and/or electronics components comprises a circuit board.

In some configurations, the circuit board is surrounded by a sealing material to prevent water and oxygen ingress into the circuit board.

In some configurations of any of the portable display units outlined in any of the above aspects, the portable display unit further comprises a dial.

In some configurations, the dial comprises a rotatable component to provide a user input, and wherein the dial comprises a push button to provide a different user input.

In some configurations, the rotatable component surrounds a periphery of the push button, and wherein a pushing surface of the push button is recessed within the rotatable component.

In a further aspect of the disclosure, in accordance with certain features, aspects and advantages of at least one of the embodiments disclosed herein, a breathing assistance apparatus with a portable display unit is disclosed.

In some configurations, the breathing assistance apparatus is a standalone humidifier apparatus, a continuous positive airway pressure (CPAP) apparatus, a high flow apparatus, or a ventilator for example.

In this specification, a ‘breathing assistance apparatus’ may also be referred to as a ‘respiratory assistance apparatus’. The breathing assistance apparatus provides a gases flow to a patient.

Features from one or more embodiments or configurations may be combined with features of one or more other embodiments or configurations. Additionally, more than one embodiment may be used together during a process of respiratory support of a patient.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

It should be understood that alternative embodiments or configurations may comprise any or all combinations of two or more of the parts, elements or features illustrated, described or referred to in this specification.

This disclosure may also be said broadly to consist in the parts, elements and features referred to or indicated in the specification of the application, individually or collectively, and any or all combinations of any two or more said parts, elements or features, and where specific integers are mentioned herein which have known equivalents in the art to which this disclosure relates, such known equivalents are deemed to be incorporated herein as if individually set forth.

The term ‘comprising’ as used in this specification means ‘consisting at least in part of’. When interpreting each statement in this specification that includes the term ‘comprising’, features other than that or those prefaced by the term may also be present. Related terms such as ‘comprise’ and ‘comprises’ are to be interpreted in the same manner.

As used herein the term ‘(s)’ following a noun means the plural and/or singular form of that noun.

As used herein the term ‘and/or’ means ‘and’ or ‘or’, or where the context allows both.

The disclosure consists in the foregoing and also envisages constructions of which the following gives examples only.

### BRIEF DESCRIPTION OF THE DRAWINGS

Specific embodiments and modifications thereof will become apparent to those skilled in the art from the detailed description herein having reference to the figures that follow, of which:

FIGS. 1A through 3D show example breathing assistance apparatuses configured to provide respiratory therapy to a user.

FIG. 4A shows schematically an additional example breathing assistance apparatus configured to provide a respiratory therapy to a user.

FIG. 4B is a front left perspective view of an example breathing assistance apparatus with a humidification chamber in position and a raised handle/lever.

FIG. 4C is an exploded view of upper and lower chassis components of a main housing of the breathing assistance apparatus of FIG. 2.

FIG. 4D is a front left side perspective view of the lower chassis of the main housing showing a heater plate assembly and other internal components.

FIG. 5 is front right perspective view of an alternative example breathing assistance apparatus.

FIG. 6 is a front right perspective view of a shroud and removable elbow of the breathing assistance apparatus.

FIG. 7 is a right side view of the removable elbow of the breathing assistance apparatus.

FIG. 8 is an overhead views of a screen carrier of a breathing assistance apparatus.

FIG. 9A is a front right perspective view of a first configuration portable display unit for use with any of the breathing assistance apparatuses.

FIG. 9B is an overhead view of the portable display unit.

FIG. 9C is a front view of the portable display unit.

FIG. 9D is a right side view of the portable display unit.

FIG. 9E is a rear view of the portable display unit.

FIG. 9F is a rear underside perspective view of the portable display unit.

FIG. 10 is a right side sectional view of the portable display unit.

FIG. 11A is a front right perspective view showing the portable display unit connected to the screen carrier of a base unit of a breathing assistance apparatus.

FIG. 11B is a front right perspective view showing a connection feature on the screen carrier.

FIG. 12 is a view similar to FIG. 11A but showing a cable for tethering the portable display unit with the base unit of the breathing assistance apparatus.

FIG. 13 is a view similar to FIG. 11A but schematically showing a change of the angular orientation of the screen of the portable display unit relative to the portable display unit housing, or schematically showing a change of the angular orientation of the portable display unit housing relative to the connection feature.

FIG. 14A shows a removable cover for the portable display unit.

FIG. 14B shows exemplary securing features of the removable cover.

FIG. 15A is a front right perspective view of an alternative portable display unit.

FIG. 15B is an overhead view of the portable display unit.

FIG. 15C is a right side view of the portable display unit.

FIG. 15D is a front view of the portable display unit.

FIG. 15E is a rear view of the portable display unit.

FIG. 15F is a front underside perspective view of the portable display unit with the support feature in a first position.

FIG. 16A is a front right perspective view showing the portable display unit connected to the screen carrier of a base unit of a breathing assistance apparatus.

FIG. 16B is a front right perspective view showing a connection feature on the screen carrier.

FIG. 17 is an underside perspective view of the portable display unit with the support feature in a second position.

FIG. 18A shows the portable display unit supported on a first support surface.

FIG. 18B shows the portable display unit supported on a second support surface.

FIG. 18C shows the portable display unit supported on a horizontal support surface.

FIG. 19 shows an exemplary use of one of the portable display units.

FIG. 20 shows other exemplary uses of one of the portable display units.

FIG. 21 shows an exemplary configuration of a breathing assistance apparatus with two display units.

FIG. 22 shows an exemplary use of a breathing assistance apparatus with two display units.

FIG. 23 is a front right perspective view of an alternative portable display unit.

FIG. 24 is an enlarged front right perspective view of a dial and buttons of the portable display unit.

FIG. 25 is a cross-sectional view of the dial.

FIG. 26 is a rear perspective view of the portable display unit.

FIG. 27 is an exploded side view of the portable display unit.

FIG. 28 is a rear overhead perspective view of a first configuration of a notification light of the portable display unit.

FIG. 29 is a front overhead perspective view of a second configuration of a notification light of the portable display unit.

FIG. 30 is a perspective sectional view showing the engagement of the notification light with the display unit housing.

FIG. 31 is a front right perspective view of an alternative breathing assistance apparatus base unit and inspiratory conduit.

FIG. 32 is an exploded front right perspective view of components of the base unit and inspiratory conduit.

FIG. 33 is a left front perspective view of part of the base unit including a display mounting component.

FIG. 34 is a rear right perspective view of the conduit and an interconnect circuit board of the base unit.

FIG. 35 is a view of a first configuration interconnect circuit board and light elements.

FIG. 36 is a view of a second configuration interconnect circuit board and light elements.

FIG. 37 is a front left perspective view of the base unit and inspiratory conduit with the portable display unit connected to the base unit.

FIG. 38 is a rear perspective view of an overmoulded portion that connects the cable to the portable display unit.

FIG. 39 is a front perspective view of the overmoulded portion and part of the cable.

FIG. 40 is a perspective view of a slot in the display unit housing for receipt of the overmoulded portion.

FIG. 41 is a side sectional view of part of the base unit and connected portable display unit, showing cable connection features on the base unit.

FIG. 42 is a rear right overhead perspective view of part of the base unit showing the cable connection features.

FIG. 43 is a front right perspective view of a display mounting component of the base unit.

FIG. 44 is a front right overhead perspective view of the base unit showing alternative connection features.

FIG. 45 is a front left overhead perspective view of the base unit showing alternative connection features.

### DETAILED DESCRIPTION

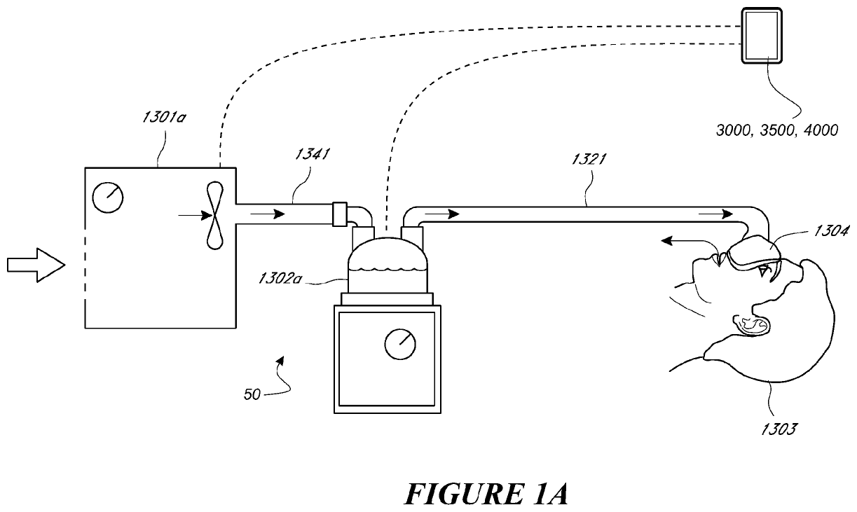

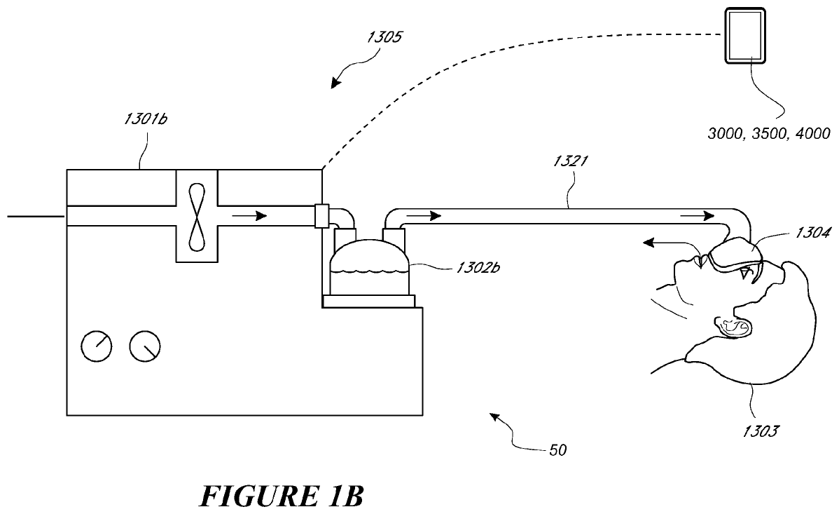

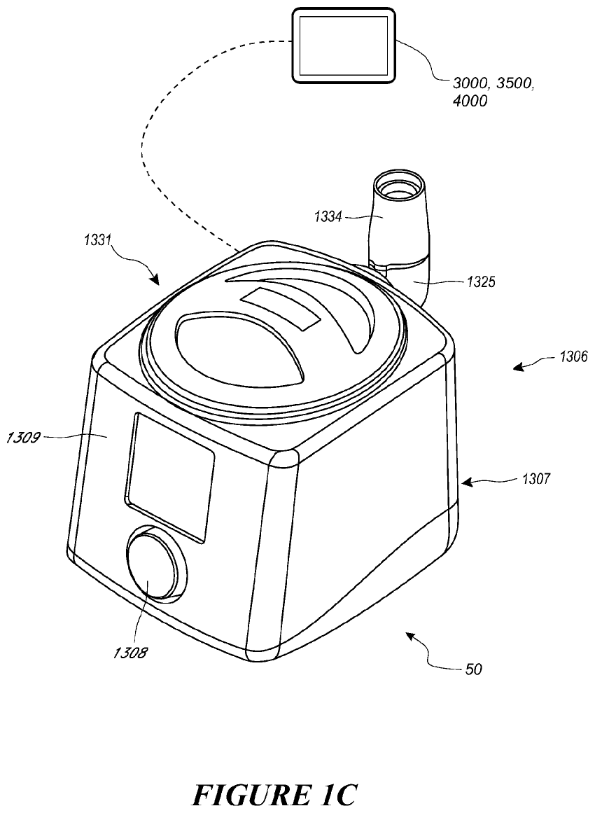

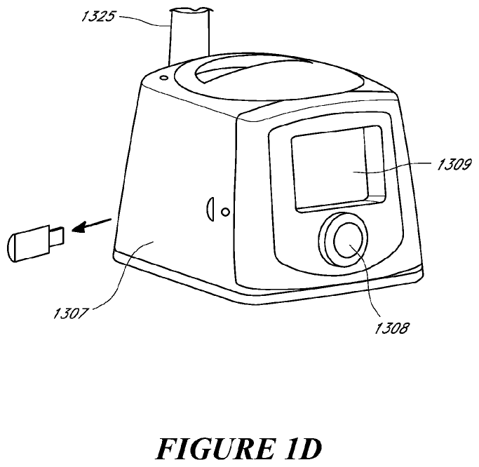

FIGS. 1A through 1D show example breathing assistance apparatuses or respiratory devices that can implement the features described herein. Each of the various systems described in FIGS. 1A-1D can be used with the portable display units 3000, 3500, 4000 described later in this specification.

The exemplary breathing assistance apparatuses can each be considered to have a base unit and other components (e.g. a patient interface), and the base unit is represented in each example by reference numeral 50.

A main usage problem with a conventional breathing assistance apparatus is that that the breathing assistance apparatus has an integrated display that presents various therapy related data and/or patient related data (e.g. respiratory rate of the patient, SpO2 readings, flow rate delivered by the apparatus and/or to the patient, pressure delivered by the apparatus and/or to the patient, humidity level or dew point, temperature of the flow of gases) to a user or clinician, and may allow for the user or clinician to adjust therapy parameters or other settings of the breathing assistance apparatus.

Many conventional breathing assistance apparatuses have an integrated humidifier having a chamber of water. For safe use, the humidifier is positioned below a patient e.g. below the bed level of the patient, or below the head of the patient. This positioning is to ensure any condensate formed in the tubes or conduits runs back into the humidifier, and does not run toward the patient or back into the flow generator. Condensate going toward the patient can cause discomfort due to liquid condensate entering the patient interface and/or patient's airways and/or in the worst case could enter the lungs of the patient. Similarly, liquid condensate running back to the flow generator can damage electronics in the flow generator or cause short circuits or cause irreparable damage to the flow generator, which can interrupt therapy. Interruption in therapy is adverse for a patient.

For safety, the breathing assistance apparatus base unit is positioned below the bed so that the breathing assistance apparatus base unit with integrated humidifier is the lowest point within a breathing flow path (i.e. a breathing circuit). In conventional breathing assistance apparatuses this means the integrated display is often too low to be viewed by a clinician. The information on the screen should be easy to view by a user or clinician, however the positioning of the device, and thus the integrated screen, can make it difficult or challenging for a user or clinician to view the therapy data and/or adjust therapy parameters. This can make usability of the breathing assistance apparatus difficult.

Conventional breathing assistance apparatuses having an integrated display require the clinician to be in close proximity to the patient to monitor and adjust therapy parameters. This can increase the risk of infectious diseases being transmitted from the patient to the clinician. By removing the display unit from the base unit of the breathing assistance apparatus as disclosed herein, the display unit can be placed away from the close proximity of the patient, such as in a separate room, from which the therapy can be monitored and adjusted by the clinician. This allows for the spread of infectious diseases to be controlled within both hospital and home settings.

Another problem with conventional devices is that the display being integrated with the breathing assistance apparatus base unit means the screen orientation is dependent on the position of the breathing assistance apparatus base unit. In a hospital there are often multiple pieces of medical equipment that are used simultaneously. The breathing assistance apparatus is often positioned away/out of the way so other equipment can be used on the patient. Therefore, the integrated display can be hidden or positioned at an angle that is difficult to view for a user or clinician. The data presented on the screen can be missed or not viewed due to the positioning.

Similarly, a conventional at home breathing assistance apparatus with an integrated display needs to be positioned below the patient and somewhere visible so the patient can see information presented on the screen. This limits the usability as the apparatus needs to be located within view of the patient.

In some of the figures, the portable display unit 3000, 3500, 4000 is represented in broken lines as being tethered to the breathing assistance apparatuses. The portable display unit 3000, 3500, 4000 may be tethered to the breathing assistance apparatus physically, physically for data communication, and/or wirelessly for data communication.

The portable display unit 3000, 3500, 4000 may be physically tethered to the breathing assistance apparatus by a cable 3061, 4061.

The portable display unit 3000, 3500, 4000 may be wirelessly tethered for one or two way data communication. The wireless communication may be any short range wireless coupling. Any suitable short range communication protocols can be used to link the base unit 50 with the portable display unit 3000, 3500, 4000. Examples include Bluetooth, ZigBee, or any other suitable protocol.

The portable display unit 3000, 3500, 4000 may be paired to the base unit 50. The portable display unit 3000, 3500, 4000 could be pre-paired with the base unit 50 for example upon manufacture, such as at a factory, such that only that portable display unit 3000, 3500, 4000 and that base unit 50 are able to be connected via the wireless communications. The portable display unit 3000, 3500, 4000 and/or the base unit 50 may have a unique connection identifier which mean that only that portable display unit 3000, 3500, 4000 and that base unit 50 are able to be connected via the wireless communications.

Alternatively, the portable display unit 3000, 3500, 4000 may be selectively/removably pairable with the base unit, 50 such that multiple portable display units 3000, 3500, 4000 may be able to be paired with the base unit 50, and portable display units 3000, 3500, 4000 may be removed from connection with the base unit 50 and paired with other base units 50. The wireless communications may also enable other devices to be connected to the base unit such as smartphones, tablets, or laptop computers for example.

In this description, data is information that has been translated into a form that is efficient for movement or processing. Relative to today's computing devices and transmission media, data is information converted into binary digital form. It is acceptable for data to be used as a singular subject or a plural subject.

The data may comprise or consist of passive information that is acted on in some way, e.g. stored, transmitted, and/or gathered. The data may comprise or consist of control signals. Controls signals are active and cause a change in behaviour of an apparatus, device, or object.

In some configurations, the data can comprise or consist of data signals representing information to be displayed on a screen of the portable display unit 3000, 3500, 4000.

The tethering may provide two way data communication so that a user can control operation of the base unit 50 via the portable display unit 3000, 3500, 4000.

In some configurations, data signals and/or control signals are transmitted from the breathing assistance apparatus to the portable display unit 3000, 3500, 4000. In some configurations, data signals and/or control signals are transmitted from the portable display unit 3000, 3500, 4000 to the breathing assistance apparatus. In some configurations, power is transmitted from the breathing assistance apparatus to the portable display unit 3000, 3500, 4000. Any combination may be possible.

Turning to FIG. 1A, a schematic view of a user 1303 receiving air from a breathing assistance apparatus comprising a modular assisted breathing unit and humidifier system is shown. A conduit 1341 provides pressurized air from an assisted breathing unit, flow generator, or blower unit 1301a to a humidifier comprising a humidification chamber 1302a. A heater plate assembly in the modular assisted breathing unit and humidifier system can be in thermal communication or contact with the humidification chamber 1302a to heat water in the chamber 1302a. Gases passing through the humidification chamber 1302a can be humidified and heated. Humidified, heated and pressurized gases exit the humidification chamber 1302a via an inspiratory conduit 1321, and are provided to the patient or user 1303 via a patient interface 1304. The patient interface 1304 shown in FIG. 1A is a nasal mask, which covers the nose of the user 1303. However, it should be noted that in systems of these types, a full face mask, nasal cannula, tracheostomy fitting, nasal pillows, oral interface, or any other suitable patient interface could be substituted for the nasal mask shown.

FIG. 1B shows a schematic view of the user 1303 receiving air from a breathing assistance apparatus comprising an integrated flow generator or blower/humidifier unit 1305. The system generally operates in the same manner as the modular system shown in FIG. 1A except that a humidification chamber 1302b of the humidifier has been integrated with a flow generator or blower unit 1301b to form the integrated unit 1305. Accordingly, the integrated blower/humidifier unit 1305 can include a heater plate assembly configured to heat water in the chamber 1302b. An example of an integrated unit is described in PCT application WO 2008/056993 and U.S. Pat. No. 8,555,879. The contents of those specifications are incorporated herein in their entirety by way of reference.

An example breathing assistance apparatus comprising an assisted breathing unit or integrated unit 1306 will now be described with reference to FIGS. 1C and 1D.

The integrated unit 1306 can include two main parts: an assisted breathing, flow generator, or blower unit 1307 and a humidifier comprising a humidification unit 1331. When in use, the humidification unit 1331, which can include a humidification chamber, generally is enclosed within an enclosure that is formed in an external casing of the integrated unit 1306. In the illustrated configuration, the top part of the humidification unit 1331 is not enclosed within the enclosure 1342. The blower unit 1307 can include a heater plate assembly in thermal communication or contact with the humidification unit 1331 to heat water inside the humidification chamber.

The flow generator or blower unit 1307 has an outer shell that generally is a rectangular block with substantially vertical side and rear walls, and a front face that is angled slightly rearwards. In the illustrated embodiment, the walls, base and top surface are all manufactured and connected as far as possible to minimise the occurrence of seams. Any necessary seams can be sealed. This outer shell generally encloses the working parts of the blower unit 1307 and forms part of the blower unit 1307.

As shown in FIGS. 1C and 1D, a user interface is located on the lower section of the front face of the illustrated integrated unit 1306 with a control display 1309 located directly above the user interface. The user interface can include a control knob 1308. A patient outlet 1325 is shown passing out of the rear wall of the integrated unit 1306. In the illustrated embodiment, in use the free end of the outlet 1325 faces upwards for ease of connection. However, the patient outlet 1325 can be rotated to one side or to the other side to move or align it in a more convenient position for storage or to provide a more convenient use position.

The illustrated patient outlet 1325 is adapted to allow both pneumatic and electrical connection to one end of a conduit, for example, the conduit (such as the inspiratory conduit 1321 of FIG. 1B), that extends between the unit 1306 and a patient interface, for example, the interface 1304 of FIG. 1B. An example of the type of connector that can be used and the type of dual connection that can be made is described in U.S. Pat. No. 6,953,354, which is hereby incorporated by reference in its entirety. It should be noted that for the purposes of reading this specification, the patient interface generally can be thought of as including both the interface 1304 and the inspiratory conduit 1321 where it would be appropriate to read it in this manner.

The integrated unit 1306 can include an inlet vent or inlet port (not shown) to draw air in from atmosphere. The inlet port or vent could also be a connector adapted to receive gases from a wall source, pressure bottle or the like. The integrated unit 1306 can also include a mechanism for providing a pressurized air flow from the inlet vent to the humidification unit 1331. The pressurized air flow mechanism can include a fan unit. The vent can be located wherever is convenient on the external surface of the integrated unit 1306. The vent can be located on the rear face of the blower unit 1307.

The air is ducted or otherwise directed along an air path through the casing of the blower unit 1307 and delivered to the humidification unit 1331, where it is humidified and heated by the heated water, before passing out of the humidification unit 1331 and onwards to the patient outlet 1325 on the blower unit 1307. The heated humidified gas then passes to the user 1303 via the inspiratory conduit 1321 and a patient interface, which can include any suitable patient interface examples disclosed herein.

The outlet port or patient outlet 1325 is adapted to enable both pneumatic attachment of the inspiratory conduit 1321 and electrical connection via an electrical connector. In FIG. 1C, a conduit connector 1334 that would normally be fitted to the end of the inspiratory conduit 1321 is shown connected to the patient outlet 1325. The outlet port or outlet connection does not have to be via the housing of the integrated unit 1306, as in the illustrated embodiment. Instead, the connection for the inspiratory conduit 1321 could be located directly on an outlet from humidification unit 1331. The illustrated form and variations generally can be referred to as connection mechanisms.

The integrated unit 1306 also contains electronic circuitry enclosed within the casing, which at least partly comprises a controller, such as a microprocessor or the like, and which provides control signals to control the output or outputs of at least the blower unit 1307, and preferably other items such as the humidification unit 1331. The control circuitry also can be adapted to receive signals from sensors in the system (for example, pressure, flow, humidity and temperature signals from these sensors as applicable) and to alter outputs from the control circuitry accordingly. The control circuitry also receives signals from user controls as the user controls are manipulated by a user and alters the output signals accordingly.

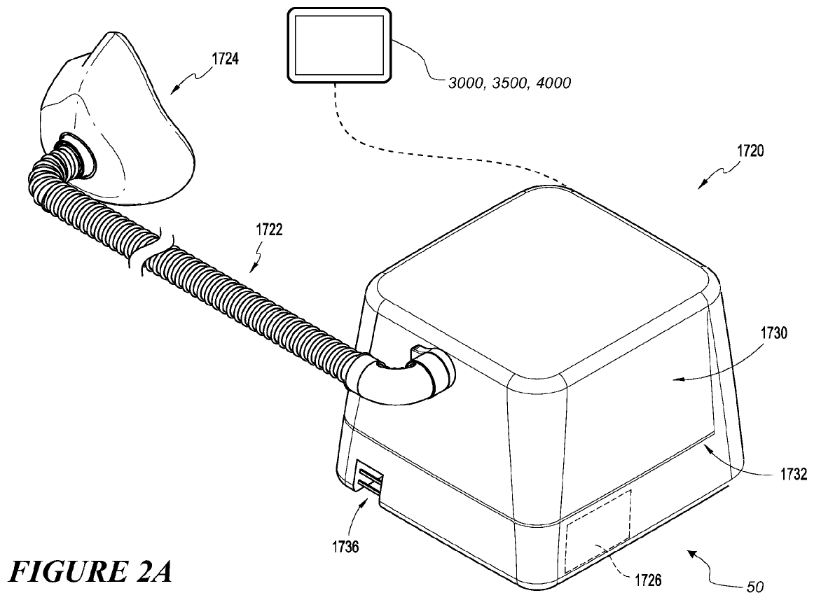

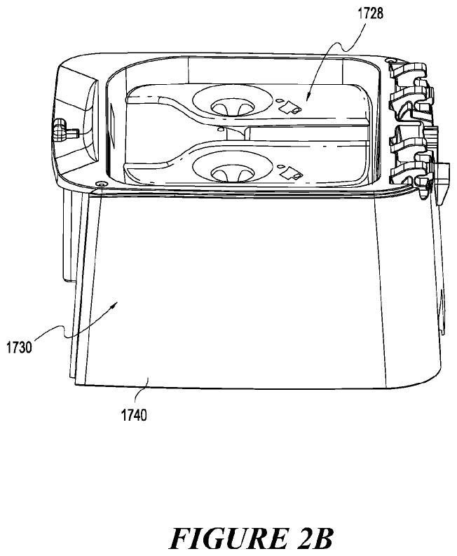

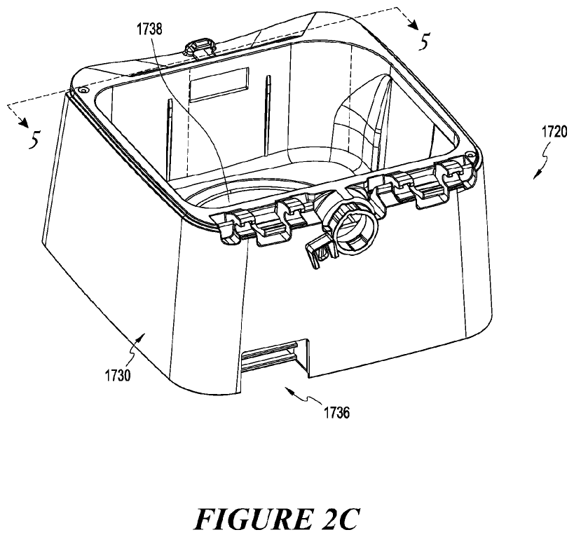

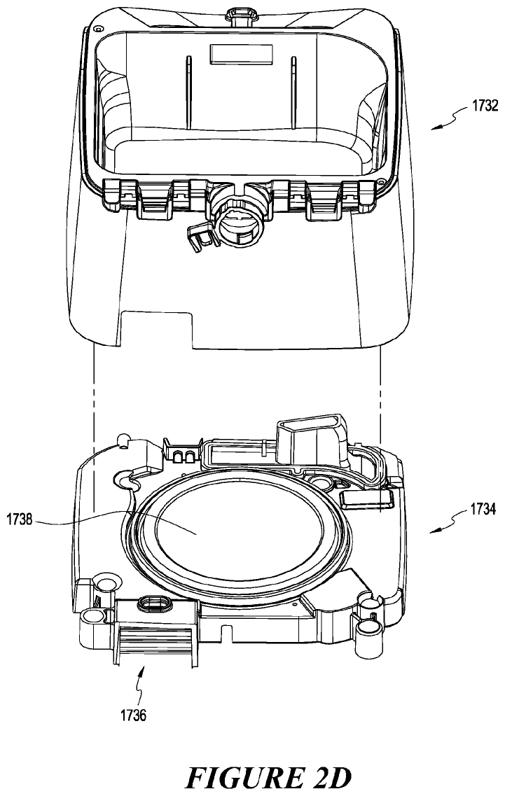

The breathing assistance apparatus shown in FIGS. 1C, 1D, and 2A-2D can be controlled by the controller to provide pressure therapy. The blower can be controlled to provide CPAP therapy or bilevel pressure therapy or any other pressure therapy. The apparatus is used with a sealed mask to provide pressure therapy, for example a nasal mask or a full face mask or other sealed mask.

FIGS. 2A through 2D show additional breathing assistance apparatuses that can implement the features described herein. Again, each of the various systems described in FIGS. 2A-2D can be used with the specific features described later in this application. Turning to FIG. 2A, a breathing assistance apparatus 1720 is shown. In the illustrated configuration, the breathing assistance apparatus 1720 is connected to an inspiratory conduit 1722 and the inspiratory conduit 1722 is connected to a patient interface 1724, such as a breathing mask or the like. Any suitable patient interface 1724 can be used.

The breathing assistance apparatus 1720 is configured to deliver a flow of pressurized breathing gases to the user through the conduit 1722 and the patient interface 1724. Accordingly, the illustrated breathing assistance apparatus 1720 can include a flow generator or blower unit 1726, which has been schematically illustrated in FIG. 2A. The blower unit 1726 can have any suitable construction. The blower unit draws ambient air into the breathing assistance apparatus 1720 and generates the flow of pressurized breathing gases.

The breathing assistance apparatus 1720 also is configured to humidify the flow of pressurized breathing gases prior to deliver to the user. Accordingly, as illustrated in FIG. 2B, the illustrated breathing assistance apparatus 1720 also can include a humidifier comprising a humidification chamber 1728. The humidification chamber 1728 can be removable from the breathing assistance apparatus 1720. Any suitable construction can be used for the humidification chamber 1728. The humidification chamber 1728 can be configured to contain a volume of liquid, such as water. The flow of pressurized breathing gases can pass over the volume of liquid en route to the user such that the flow of pressurized breathing gases can increase in humidity.

As illustrated, the breathing assistance apparatus 1720 generally can include a main body 1730. With reference to FIG. 2D, the main body 1730 can include an upper housing 1732 and a lower housing 1734. The upper housing 1732 and the lower housing 1734 can be secured together in any suitable manner. In some configurations, the bottom of the lower housing 1734 can be enclosed by a further cover.

With continued reference to FIG. 2D, the lower housing 1734 can include an air inlet 1736 through which the blower unit 1726 draws air. The blower unit 1726 can be mounted to or within the lower housing 1734. The lower housing 1734 also can support a heater plate assembly 1738. The liquid within the humidification chamber 1728 can be heated through an interaction with the heater plate assembly 1738. In some configurations, the humidification chamber 1728 can rest on a heating plate of the heater plate assembly 1738.

Other configurations are possible.

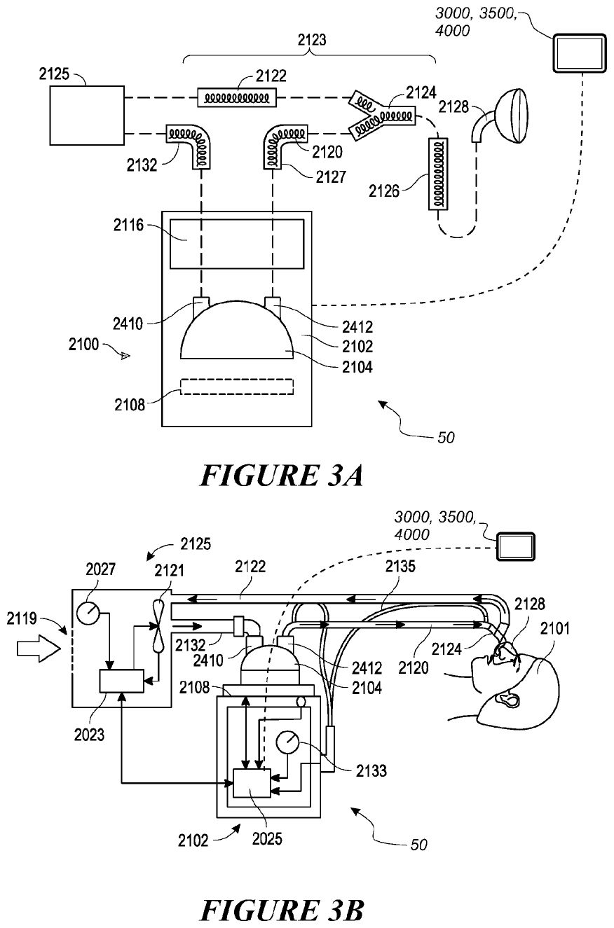

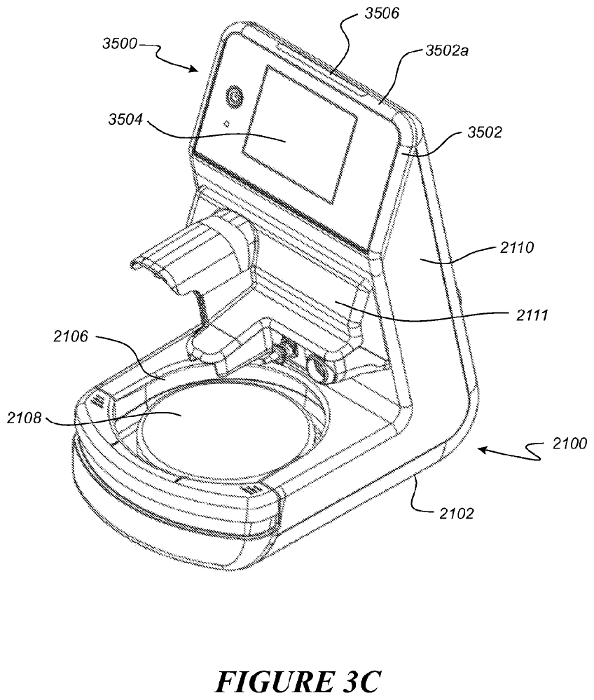

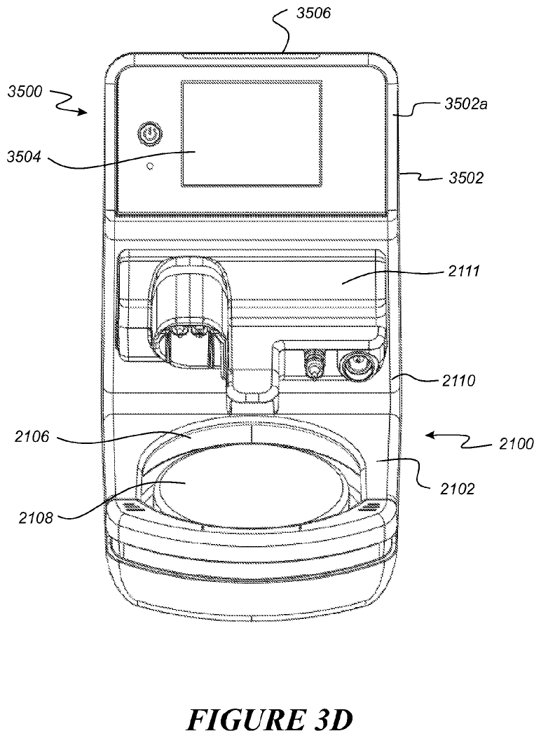

FIGS. 3A and 3B show additional breathing assistance apparatus examples that can implement the features described herein. Each of the various systems described in FIGS. 3A and 3B can be used with the specific features described later in this application. FIGS. 3A and 3B schematically illustrate examples of a humidification system or humidifier 2100 that, in some applications, can be used with breathing therapies, positive pressure apparatus, non-invasive ventilation, surgical procedures including but not limited to laparoscopy, and the like.

Desirably, the humidification system 2100 can be adapted to supply humidity or vapor to a supply of gases. The humidification system 2100 can be used with ventilators, nasal high flow systems, continuous, variable, or bi-level positive airway pressure (PAP) systems or other form of respiratory therapy. In some configurations, the humidification system 2100 can be integrated into a system that delivers any such types of therapy.

An example of the humidification system 2100 can include a heater base unit 2102 and a humidification chamber 2104. The heater base unit 2102 can comprise a heater plate assembly 2108. The humidification chamber 2104 can be configured to hold a volume of a liquid, such as water. The heater plate assembly 2108 can be configured to heat the volume of liquid held within the humidification chamber 2104 to produce vapor, either by being in thermal communication with the humidification chamber or by contacting the humidification chamber.

The humidification chamber 2104 is removable from the heater base 2102 to allow the humidification chamber 2104 to be more readily sterilized or disposed. The body of the humidification chamber 2104 can be formed from a non-conductive glass or plastics material but the humidification chamber 2104 can also include conductive components. For instance, the humidification chamber 2104 can include a highly heat-conductive base (for example, an aluminium base) contacting or associated with the heater plate assembly 2108 on the heater base unit 2102.

The heater base unit 2102 can also include electronic controls. In this example, the heater base unit 2102 includes a master controller 2025. The master controller 2025 can comprise an electronic, analog, or digital processor or controller. Preferably, the master controller 2025 comprises a microprocessor-based controller configured to execute computer software commands stored in associated memory. In response to user-set humidity or temperature values input via a user interface 2133, for example, and other inputs, the master controller 2025 determines when (or to what level) to energize a heating element of the heater plate assembly 2108 to heat the liquid within the humidification chamber 2104.

In some configurations, a humidification system 2100 comprises a humidifier comprising the heater base unit 2102 and humidification chamber 2104, an inspiratory conduit 2120, and optionally a patient interface 2128. In some configurations, the humidification system 2100 can comprise an expiratory conduit.

The humidifier can be used in various types of therapies e.g. invasive ventilation, non-invasive ventilation, nasal high flow, and CPAP for example. The humidifier is a standalone unit that is used with a gases supply e.g. wall gases source or a ventilator or other gases source.

The humidifier will typically be positioned below patient bed level to allow any condensate that may form in the inspiratory conduit and/or patient interface to run down into the humidifier.

The humidification system 2100 also can include a gases supply 2125. In some configurations, the gases supply 2125 can comprise a flow generator, ventilator, blower unit, or any other suitable source of pressurized gases suitable for breathing or use in medical procedures. The gases supply 2125 can be separate from or combined with the heater base 2102. For example, as shown in FIG. 3B, dry or relatively dry gases, or ambient air, enter the gases supply 2125 through a vent 2119. A fan 2121 can improve gas flow into the gases supply by drawing air or other gases through the vent 2119. The fan 2121 can be, for instance, a variable speed fan, where a controller 2023 controls the fan speed. In particular, the function of the controller 2023 can be controlled by the master controller 2025 in response to inputs from the master controller 2025 and a user-set predetermined required value (preset value) of pressure or fan speed via a dial 2027.

The humidification system also can include a breathing circuit 2123 in fluid communication with the humidifier. The breathing circuit 2123 can include an inspiratory conduit 2120. A chamber end of the inspiratory conduit 2120 can be configured to connect to an outlet port 2412 of the humidification chamber 2104. A patient end of the inspiratory conduit 2120 can be configured to connect to the patient, for example, via a patient interface 2128 to deliver gases to a patient. In some configurations, the inspiratory conduit 2120 can be coupled directly to the patient interface 2128. Any suitable type of the patient interface 2128 can be incorporated. Patient interface is a broad term and is to be given its ordinary and customary meaning to a person of ordinary skill in the art (that is, it is not to be limited to a special or customized meaning) and includes, without limitation, masks (such as tracheal masks, face masks and nasal masks), cannulas, and nasal pillows.

A temperature probe 2135 can connect to the inspiratory conduit 2120 near the patient interface 2128, or directly to the patient interface 2128. The temperature probe 2135 monitors the temperature near or at the patient interface 2128.

A heating element (not shown), for example, a heating element that is associated with the temperature probe or a heating element not associated with a temperature probe, can be used to adjust the temperature of the patient interface 2128 and/or the inspiratory tube 2120 to raise the temperature of the inspiratory conduit 2120 and/or the patient interface 2128 above the saturation temperature, thereby reducing the opportunity for unwanted condensation.

In some configurations in which the flow generator or gases supply 2125 is separate from the heater base unit 2102, the breathing circuit 2123 can include a supply conduit 2132. A gases supply end of the supply conduit 2132 can be configured to connect to an output of the gases supply 2125. A chamber end of the supply conduit 2132 can be configured to connect to an inlet port 2410 of the humidification chamber 2104.

In some configurations, such as those used with a ventilator, the breathing circuit 2123 also can include an expiratory conduit 2122. A user end of the expiratory conduit 2122 can be configured to connect to the patient interface 2128, and a gases supply end of the expiratory conduit 2122 can be configured to connect to a return of the gases supply 2125. The expiratory conduit 2122 can have a temperature probe and/or heating element, as described above with respect to the inspiratory conduit 2120, integrated with it to reduce the opportunity for condensation. Furthermore, the expiratory conduit 2122 need not return exhaled gases to the gases supply 2125. In some configurations, exhaled gases can be passed directly to ambient surroundings or to other ancillary equipment, such as an air scrubber/filter (not shown). The expiratory conduit 2122 may be omitted altogether.

As shown in FIG. 3A, the user ends of the inspiratory conduit 2120 and the expiratory conduit 2122 can be connected to each other via a Y-piece 2124. The Y-piece 2124 can be connected to a patient supply conduit 2126. In some configurations, the patient supply conduit 2126 can include a catheter mount, for example but without limitation. The patient supply conduit 2126 can be connected to the patient interface 2128. The Y-piece 2124 may couple to the patient interface 2128 without the patient supply conduit 2126 intervening.

In use, the humidification chamber 2104 is installed onto a heating plate of the heater plate assembly 2108, to be in thermal communication with the heater plate assembly or to contact the heater plate assembly. The heater plate assembly 2108 heats liquid, such as water, in the humidification chamber 2104 to produce vapor. Dry or relatively dry gases flow from the gases supply 2125, through the supply conduit 2132, and into the humidification chamber 2104 through the inlet port 2410. The gases pass over the liquid in the humidification chamber 2104 and become humidified by the vapor. Humidified gases exit the humidification chamber 2104 through the outlet port 2412 and flow through the inspiratory conduit 2120 to a patient 2101. Gases exhaled by the patient 2101 may be returned to the gases supply 2125 through the expiratory conduit 2122. Any or all of the components of the breathing circuit 2123 can include a heating element, for example, a heating wire 2127, to help maintain the gases at a desired temperature and to reduce the likelihood of significant condensation formation in the conduits.

FIGS. 3C and 3D show an example of a heater base 2102 (i.e. base unit). The heater base unit (i.e. base unit) 2102 is used as part of a humidifier or humidification system 2100 as shown in FIGS. 3A and 3B. The heater base 2102 comprises a heater plate assembly 2108, as described earlier. The heater base 2102 comprises a receptacle 2106 to receive a humidification chamber. The humidification chamber is heated by the heater plate assembly to generate humidity, as described herein.

The heater base 2102 comprises a vertical column 2110 (i.e. vertical wall) that extends from the base. A sensing module 2111 (i.e. sensor cartridge), as shown in FIGS. 3C and 3D is located on the vertical column 2110. The sensing module 2111 is removably connectable to the vertical column 2110 via one or more couplers (not shown). The sensing module comprises one or more sensors. The sensors can be inserted into the gases flow and/or interact with the chamber to sense properties of gases. In one example the sensing module 2111 (i.e. sensor cartridge) comprises at least one flow sensor and at least one temperature sensor. The module 2111 may comprise multiple flow and temperature sensors.

The heater base 2102 comprises a display unit 3500. The display unit 3500 comprises a housing 3502. The display unit 3500 comprises a display screen 3504 and one or more physical user interface features 3506 such as for example push buttons or one or more dials etc. The display screen 3504 may be a touchscreen that allows a user to input commands to the humidifier 2100 via the touchscreen display screen 3504. The housing 3502 may be removable by using an appropriate tool, by a technician e.g. a hospital technician or maintenance engineer. The housing 3502 may be removed and replaced with a different housing that engages with the display unit and/or engages with the vertical column. Alternatively the display unit 3500 may be a removable display unit as described. The display unit 3500 may be removable for replacement and/or may be removable so it can be moved to a more visible location.

The housing 3502 comprises a light bar 3506. The light bar 3506 extends along a portion of the housing. The light bar 3506 as shown in FIGS. 3C, 3D is flush with the housing 3502 and follows the shape and contour of the housing 3502. Alternatively the light bar 3506 may project outwardly from the housing. The light bar 3506 is elongate in shape, and in the illustrated example is a substantially rectangular. The light bar 3506 may alternatively be cylindrical or any other suitable shape. The light bar 3506 is preferably integrated into the housing. The light bar 3506 is centrally positioned in housing 3502, and centrally positioned relative to the display unit 3500. As shown in FIGS. 3C-3D the light bar 3506 is positioned in the housing 3502 above the display screen 3504. The light bar 3506 is positioned on a rounded portion of a periphery of the housing 3502. The light bar 3506 preferably defines a portion of an upper edge (i.e. upper peripheral region) of the housing 3502. The display unit 3500 comprises one or more lights e.g. LEDs that are located on a PCB within the display unit. The LEDs are positioned adjacent the light bar so that light from the LEDs projects out of the light bar The light bar may be white in colour or may have a specific colour. The display unit 3500 comprises multiple LEDs of different colours. The various colours are used to denote various alarm states. For example activation of a red LED in the display unit 3500 causes the light bar 3506 to light red, denoting a critical alarm or a high priority alarm. In another example activation of yellow LED in the display unit causes the light bar to light yellow, denoting a medium priority alarm. The light bar is preferably made from a plastics material that allows projection of light or other suitable materials such as glass or quartz or crystal. The LEDs may be mounted on an internal PCB. Optionally the display 3500 may comprise one or more light pipes that direct light from the LED to the light bar such that the light from the LEDs is projected out of the light bar 3506.

In an alternative construction, the display unit 3500 may comprise a plurality of light bars disposed on the housing. The light bars may be equally spaced or may be unequally spaced. The light bars may be positioned at the top or upper section of the housing 3502 or may be positioned at various locations around the housing. In a further alternative the housing 3502 comprises bezel (i.e. a peripheral casing) 3502a that surrounds the display screen 3504. The bezel 3502a may be removable and replaceable. The bezel 3502a (i.e. peripheral casing) may be removed by an appropriate tool. Multiple bezels may be used with the display unit for example, a bezel with an integrated light bar 3506 or a bezel with no light bar or a bezel with multiple light bars.

The light bar 3506 is advantageous because it makes the display unit 3500 more visible and makes communication of information to a user e.g. a clinician simpler. The light bar makes alarm communication simpler. This is useful as humidifiers as positioned below a bed level of the patient i.e. at the lowest point. Further when the removable display unit with the light bar also makes communication of information e.g. alarm information simpler and more clear.

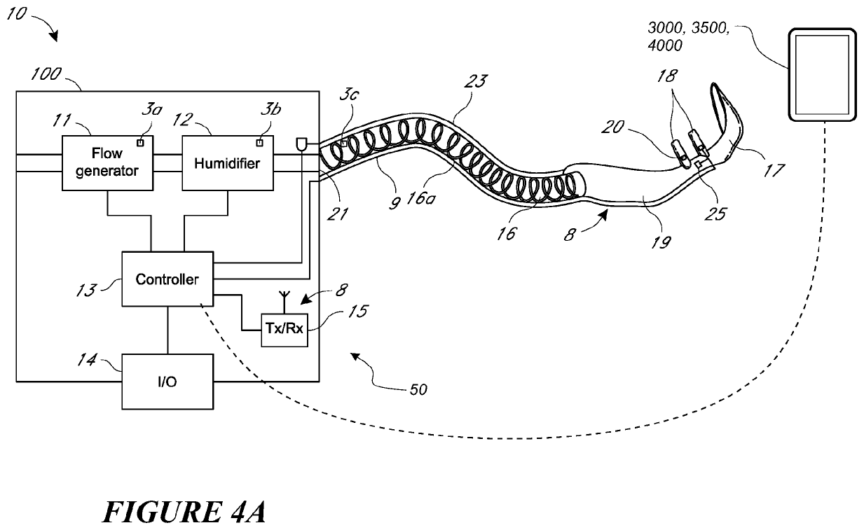

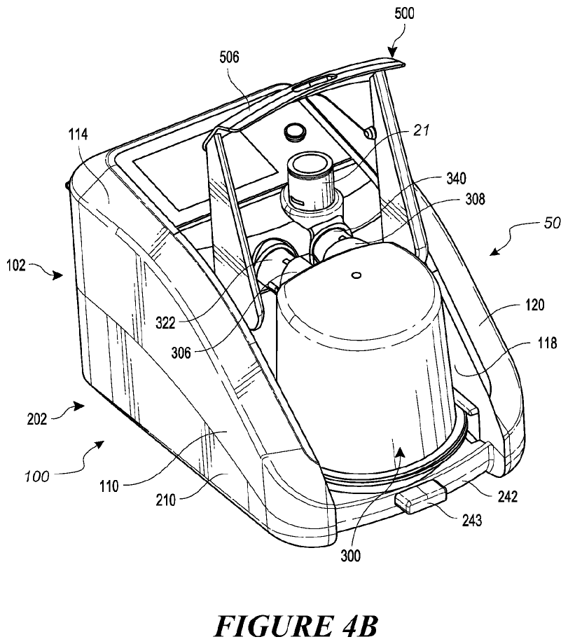

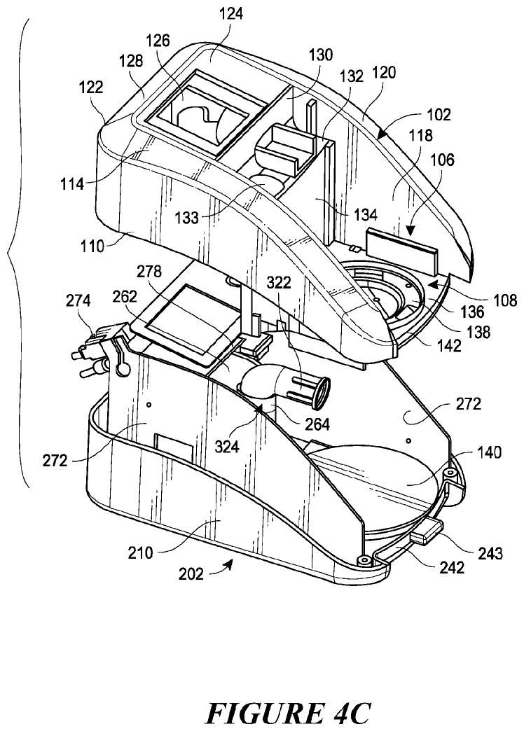

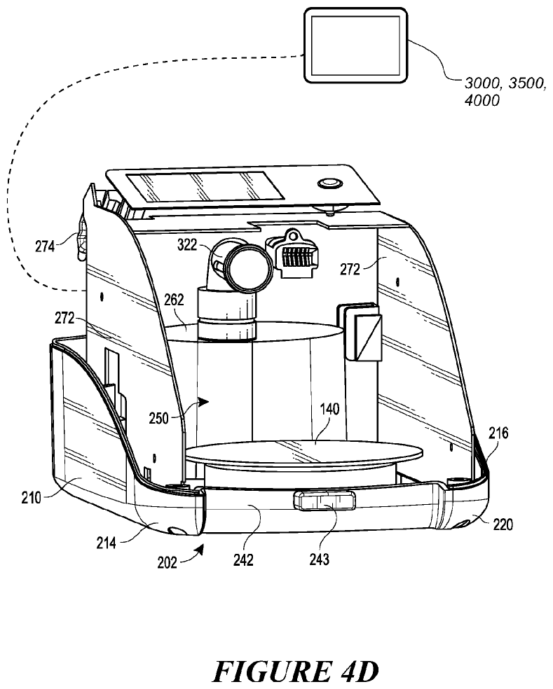

A schematic representation of an example breathing assistance apparatus 10 is provided in FIG. 4A. The apparatus 10 could, for example, be a CPAP apparatus or a high flow apparatus. An exemplary CPAP apparatus is described in WO 2011/056080 and U.S. Pat. No. 11,110,246. The contents of those specifications are incorporated herein in their entirety by way of reference.

A CPAP apparatus is a gases supply and optionally gases humidification apparatus. The apparatus is operable to provide respiratory assistance to patients or users who require a supply of gas (humidified or otherwise) at positive pressure for the treatment of diseases such as Obstructive Sleep Apnea (OSA), snoring, or Chronic Obstructive Pulmonary Disease (COPD) and the like. Bilevel pressure therapy may be used to treat COPD or other respiratory conditions. A CPAP apparatus would typically include a humidification chamber containing liquid, so as to form a combined assisted breathing unit and humidifier.

CPAP apparatuses, when used with a humidifier, typically have a structure where gases at a required pressure are delivered from an assisted breathing unit or blower unit to a humidification chamber downstream from the blower. As the gases pass through the humidification chamber, they become saturated with liquid vapour (e.g. water vapour). A flexible tubular gases conduit delivers the gases to a user or patient downstream from the humidification chamber.

The configuration shown in FIG. 4A may be a high flow apparatus. The controller of the apparatus may be configured to control the blower and humidifier of the apparatus accordingly. A high flow apparatus may be used to deliver a high gas flow or high flow therapy to a patient to assist with breathing and/or treat breathing disorders including chronic obstructive pulmonary disease (COPD), or respiratory distress syndrome or other respiratory conditions where patients struggle to breathe or have respiratory distress. The apparatus may be used for nasal high flow or tracheal high flow.

Further, nasal high flow can also be used in anaesthetic applications for example to pre-oxygenate patients prior to sedation and once patients are woken from sedation. Nasal high flow may be used for post-extubation respiratory support.

A high flow apparatus includes a gases supply and typically includes a humidification apparatus.

The breathing assistance apparatuses typically have one or more accessories such as a breathing conduit and a patient interface such as a cannula or mask for delivering gases to a patient. The conduit enables gases to be delivered from the housing of the breathing assistance apparatus to the patient. For example, the apparatus may be placed on a floor or other support surface, and the patient may be in a bed. The breathing assistance apparatus may have a recess for receipt of a humidification chamber. The humidification chamber will receive liquid from, for example, a flexible liquid bag that delivers liquid to a humidification chamber via one more tubes. Alternatively, the humidification chamber can be removed and refilled as required. The recess will contain a heater plate to heat the humidification chamber, to humidify gases passing through the humidification chamber. The humidified gases are then delivered to the patient.

The breathing assistance apparatus 10 can include a main device housing 100 which can be part of by a base unit of the system. The main device housing 100 can contain a flow generator or blower unit 11 that can be in the form of a motor/impeller arrangement, an optional humidifier comprising a humidification chamber 12, a controller 13, and a user interface 14. As discussed below, the user interface may be provided by a portable display unit 3000, 3500, 4000. The user interface 14 used for this or any other described breathing assistance apparatuses can include a display and input device(s) such as button(s), a touch screen, a combination of a touch screen and button(s), or the like. The controller 13 can include one or more hardware and/or software processors and can be configured or programmed to control the components of the apparatus, including but not limited to operating the blower unit 11 to create a flow of gases for delivery to a patient, operating the humidification chamber 12 (if present) to humidify and/or heat the gases flow, receiving user input from the user interface 14 for reconfiguration and/or user-defined operation of the respiratory system 10, and outputting information (for example on the display) to the user. The user can be a patient, healthcare professional, or others.

With continued reference to FIG. 4A, an inspiratory conduit 16 can be coupled to a gases flow outlet 21 in the main device housing 100 of the respiratory system 10, and be coupled to a patient interface 17, such as a non-sealing interface like a nasal cannula with a manifold 19 and nasal prongs 18. The inspiratory conduit 16 can also be coupled to a face mask, a nasal mask, a nasal pillow mask, an unsealed tracheostomy interface, or others.

The gases flow can be generated by the blower unit 11, and may be humidified, before being delivered to the patient via the inspiratory conduit 16 through the patient interface 17. The controller 13 can control the blower unit 11 to generate a gases flow of a desired flow rate, and/or one or more valves to control mixing of air and oxygen or other breathable gas. The controller 13 can control a heating element beneath the humidification chamber 12 to heat the gases to a desired temperature that achieves a desired level of temperature and/or humidity for delivery to the patient. The inspiratory conduit 16 can have a heating element 16a, such as a heater wire, to heat gases flow passing through to the patient. The heating element 16a can also be under the control of the controller 13.

The apparatus 10 can use ultrasonic transducer(s), thermistor(s), pressure sensor(s), temperature sensor(s), humidity sensor(s), or other sensors, in communication with the controller 13, to monitor characteristics of the gases flow and/or operate the apparatus 10 in a manner that provides suitable therapy. The gases flow characteristics can include gases' concentration, flow rate, pressure, temperature, humidity, or others. The sensors 3a, 3b, 3c, 20, 25, such as flow, temperature, humidity, and/or pressure sensors, can be placed in various locations in the main device housing 100, the inspiratory conduit 16, and/or the patient interface 17. The controller 13 can receive output from the sensors to assist it in operating the respiratory system 10 in a manner that provides suitable therapy, such as to determine a suitable target temperature, flow rate, and/or pressure of the gases flow. Providing suitable therapy can include meeting a patient's inspiratory demand.

The apparatus 10 can include a wireless data transmitter and/or receiver, or a transceiver 15 to enable the controller 13 to receive data 8 in a wireless manner from the operation sensors and/or to control the various components of the apparatus 10. Additionally, or alternatively, the transmitter and/or receiver 15 can deliver data to a remote server. Additionally, or alternatively, the data transmitter and/or receiver 15 can receive data from the portable display unit 3000, 3500, 4000 and/or can transmit data to the portable display unit 3000, 3500, 4000. The data may comprise or consist of control signals. That can enable remote control of the apparatus 10 by the portable display unit 3000, 3500, 4000 and/or can provide data and/or control signals to the portable display unit 3000, 3500, 4000 for display and user feedback.

The apparatus 10 can additionally include a wired connection, for example, using one or more cables or wires, to enable the controller 13 to receive data and/or control signals 8 from the operation sensors and/or to control the various components of the apparatus 10. For example, the apparatus 10 can use the wired connection to transmit power from the base unit of the apparatus to the portable display unit 3000, 3500, 4000 and/or to transmit data from the base unit to the portable display unit 3000, 3500, 4000 and data from the portable display unit 3000, 3500, 4000 to the controller 13 of the base unit. As outlined above, the data may comprise or consist of control signals.

The breathing assistance apparatus 10 can be used in a variety of applications. For instance, the apparatus 10 can be any of the following breathing assistance apparatuses or respiratory apparatuses: a continuous positive air pressure (CPAP) device, a ventilator, a humidifier, a high flow therapy device, a surgical humidifier (for example, an insufflator), combinations of the same, or the like.

CPAP treatment of obstructive sleep apnea involves the delivery of pressurized, breathable gas, usually air, to a user's airways using an inspiratory conduit and a patient interface, such as a mask. The gas pressures employed for CPAP typically range from about 4 cm H2O to about 28 cm H2O at flow rates of up to about 180 L/min (measured at the patient interface), depend upon the requirements of the user. The pressurized gas acts as a pneumatic splint for the airway of the user. As such, the pressurized gas reduces the likelihood of collapsing of the airway.

The breathing assistance apparatus 10 may be a high flow therapy apparatus. High flow therapy as discussed herein is intended to be given its typical ordinary meaning as understood by a person of skill in the art, which generally refers to a respiratory assistance system delivering a targeted flow of humidified respiratory gases via an intentionally unsealed patient interface with flow rates generally intended to meet or exceed inspiratory flow of a patient. Typical patient interfaces include, but are not limited to, a nasal or tracheal patient interface. Typical flow rates for adults often range from, but are not limited to, about fifteen liters per minute (LPM) to about seventy liters per minute or greater. Typical flow rates for pediatric patients (such as neonates, infants and children) often range from, but are not limited to, about one liter per minute per kilogram of patient weight to about three liters per minute per kilogram of patient weight or greater. High flow therapy can also optionally include gas mixture compositions including supplemental oxygen and/or administration of therapeutic medicaments. High flow therapy is often referred to as nasal high flow (NHF), humidified high flow nasal cannula (HHFNC), high flow nasal oxygen (HFNO), high flow therapy (HFT), or tracheal high flow (THF), among other common names. The flow rates used to achieve ‘high flow’ may be any of the flow rates listed below.

High flow therapy may be administered to the nares of a user and/or orally, or via a tracheostomy interface. High flow therapy may deliver gases to a user at a flow rate at or exceeding the intended user's peak inspiratory flow requirements. The high flow therapy may generate a flushing effect in the nasopharynx such that the anatomical dead space of the upper airways is flushed by the high incoming gases flow. This can create a reservoir of fresh gas available for each and every breath, while minimizing re-breathing of nitrogen and carbon dioxide. Meeting inspiratory demand and flushing the airways is additionally important when trying to control the patient's FdO2. High flow therapy can be delivered with a non-sealing patient interface such as, for example, a nasal cannula. The nasal cannula may be configured to deliver breathing gases to the nares of a user at a flow rate exceeding the intended user's peak inspiratory flow requirements.

Nasal high flow provides dynamic pressure to a patient in synchrony to the breathing of the patient. For example, nasal high flow being provided to a patient can increase pressure during the expiratory phase of a patient. This can reduce the respiratory rate of the patient and reduce respiratory effort of the patient. Reduced respiratory effort and respiratory rate are helpful to a patient with respiratory conditions e.g. COPD.

The term ‘non-sealing patient interface’ as used herein can refer to an interface providing a pneumatic link between an airway of a patient and a gases flow source (such as from flow generator 11) that does not completely occlude the airway of the patient. Non-sealed pneumatic link can comprise an occlusion of less than about 95% of the airway of the patient. The non-sealed pneumatic link can comprise an occlusion of less than about 90% of the airway of the patient. The non-sealed pneumatic link can comprise an occlusion of between about 40% and about 80% of the airway of the patient. The airway can include one or more of a nare or mouth of the patient. For a nasal cannula the airway is through the nares.

FIGS. 4B through 4D show an example breathing assistance apparatus embodying the apparatus 10 having a base unit 50 with a main housing 100, which can implement the features described herein. The main housing 100 has a main housing upper chassis 102 and a main housing lower chassis 202. The main housing upper chassis 102 has a peripheral wall arrangement 106 (see FIG. 4C). The peripheral wall arrangement defines a humidification chamber bay 108 for receipt of a removable humidification chamber 300 to form a humidifier. The removable humidification chamber 300 contains a suitable liquid such as water for humidifying gases that can be delivered to a patient.

In the form shown, the peripheral wall arrangement 106 of the main housing upper chassis 102 can include a substantially vertical left side outer wall 110 that is oriented in a front-to-rear direction of the main housing 100, a substantially vertical left side inner wall 112 that is oriented in a front-to-rear direction of the main housing 100, and an interconnecting wall 114 that extends between and interconnects the upper ends of the left side inner and outer walls 110, 112.

The main housing upper chassis 102 can further include a substantially vertical right side outer wall 116 that is oriented in a front-to-rear direction of the main housing 100, a substantially vertical right side inner wall 118 that is oriented in a front-to-rear direction of the main housing 100, and an interconnecting wall 120 that extends between and interconnects the upper ends of the right side inner and outer walls 116, 118. The interconnecting walls 114, 120 are angled towards respective outer edges of the main housing 100 but can alternatively be substantially horizontal or inwardly angled.

The main housing upper chassis 102 can further include a substantially vertical rear outer wall 122. An upper part of the main housing upper chassis 102 can include a forwardly angled surface 124. The surface 124 can have a recess 126 for receipt of a display and user interface module 14. The display can be configured to display characteristics of sensed gas(es) in real time. An interconnecting wall 128 can extend between and interconnect the upper end of the rear outer wall 122 and the rear edge of the surface 124.

A substantially vertical wall portion 130 can extend downwardly from a front end of the surface 124. A substantially horizontal wall portion 132 can extend forwardly from a lower end of the wall portion 130 to form a ledge. A substantially vertical wall portion 134 can extend downwardly from a front end of the wall portion 132 and terminate at a substantially horizontal floor portion 136 of the humidification chamber bay 108. The left side inner wall 112, right side inner wall 118, wall portion 134, and floor portion 136 together can define the humidification chamber bay 108. The floor portion 136 of the humidification chamber bay 108 can have a recess 138 to receive a heater arrangement such as a heater plate assembly 140 or other suitable heating mechanisms for heating liquid in the humidification chamber 300 for use during a humidification process.

The main housing lower chassis 202 can be attachable to the upper chassis 102, either by suitable fasteners or integrated attachment features such as clips for example. The main housing lower chassis 202 can include a substantially vertical left side outer wall 210 that is oriented in a front-to-rear direction of the main housing 100 and is contiguous with the left side outer wall 110 of the upper chassis 102, and a substantially vertical right side outer wall 216 that is oriented in a front-to-rear direction of the main housing 100 and is contiguous with the right side outer wall 116 of the upper chassis 102. The main housing lower chassis 202 can further include a substantially vertical rear outer wall 222 that is contiguous with the rear outer wall 122 of the upper chassis 102.

The lower housing chassis 202 can have a lip 242 that is contiguous with the lip 142 of the upper housing chassis 102, and also forms part of the recess for receiving the handle portion 506 of the lever 500. The lower lip 242 can include a forwardly directed protrusion 243 that acts as a retainer for the handle portion 506 of the lever 500. Instead of the lever 500, the system can have a spring loaded guard to retainer the humidification chamber 300 in the humidification chamber bay 108.

An underside of the lower housing chassis 202 can include a bottom wall 230. Respective interconnecting walls 214, 220, 228 can extend between and interconnect the substantially vertical walls 210, 216, 222 and the bottom wall 230. The bottom wall 230 can include a grill 232 comprising a plurality of apertures to enable drainage of liquid in case of leakage from the humidification chamber 300 (for example from spills). The bottom wall 230 additionally can include elongated forward-rearward oriented slots 234. The slots 234 can additionally enable drainage of liquid in case of leakage from the humidification chamber 300, without the liquid entering the electronics housing. In the illustrated configuration, the slots 234 can be wide and elongate relative to the apertures of the grill 232 to maximize the drainage of liquid.

The lower chassis 202 can have a motor recess 250 for receipt of a motor module. The motor module may comprise one or more sensors for sensing parameters of gas flow through the motor. The motor module can be removable or not removable from the main housing 100 (not shown). All of the walls and the ceiling 262 can be continuous, gas impermeable, and unbroken other than the gases flow passage. Therefore, the entire motor recess 250 can be gas impermeable and unbroken, other than the gases flow passage.

The motor module can be insertable into the recess 250 and attachable to the lower chassis 202. Upon insertion of the motor module into the lower chassis 202, the gases flow passage tube 264 can extend through the downward extension tube 133 and be sealed by the soft seal.

The humidification chamber 300 can be fluidly coupled to the base unit of the apparatus 10 in a linear slide-on motion in a rearward direction of the humidification chamber 300 into the humidification chamber bay 108, from a position at the front of the housing 100 in a direction toward the rear of the housing 100. A gases outlet port 322 can be in fluid communication with the motor.

The humidification chamber gases inlet port 306 can be complementary with the gases outlet port 322, and the humidification chamber gases outlet port 308 can be complementary with the gases inlet port 340. The axes of those ports can be parallel to each other to enable the humidification chamber 300 to be inserted into the humidification chamber bay 108 in a linear movement. The respiratory device can have air and oxygen (or alternative auxiliary gas) inlets in fluid communication with the motor to enable the motor to deliver air, oxygen (or alternative auxiliary gas), or a mixture thereof to the humidification chamber 300 and thereby to the patient.

The main housing 100 of the base unit forms a common housing for the flow generator/motor and the humidifier.

As shown schematically in FIG. 4D, the lower housing chassis 202 can include suitable electronics board(s) 272. The electronic(s) boards 272 will typically be circuit board(s). At least one of the electronics board(s) 272 may comprise a main circuit board of the base unit 50. The, or at least one of, the circuit boards 272 may comprise sensing circuit board(s). The circuit board(s) 272 may comprise printed circuit board(s) (PCB).

In some configurations, the circuit board(s) 272 may be provided between an inner casing portion and an outer casing portion.

The electronics boards can be positioned adjacent respective outer side walls 210, 216 of the lower housing chassis 202. The electronics boards 272 can contain, or can be in electrical communication with, suitable electrical or electronics components, such as but not limited to microprocessors, capacitors, resistors, diodes, operational amplifiers, comparators, and switches. Sensors can be used with the electronic boards 272. Components of the electronics boards 272 (such as but not limited to one or more microprocessors) can act as the controller 13 of the apparatus.

One or both of the electronics boards 272 can be in electrical communication with the electrical components of the apparatus 10, including the display unit and user interface 14, motor, valve and the heater plate assembly 140, to operate the motor to provide the desired flow rate of gases, operate the humidification chamber 12 to humidify and heat the gases flow to an appropriate level, and supply appropriate quantities of oxygen (or quantities of an alternative auxiliary gas) to the gases flow.

The electronics boards 272 can be in electrical communication with a connector arrangement 274 projecting from the rear wall 122 of the upper housing chassis 102. The connector arrangement 274 may be coupled to an alarm, pulse oximetry port, and/or other suitable accessories. The electronics boards 272 can also be in electrical communication with an electrical connector 276 that can also be provided in the rear wall 122 of the upper housing chassis 102 to provide mains or battery power to the components of the device.

As mentioned above, operation sensors, such as flow, temperature, humidity, and/or pressure sensors can be placed in various locations in the respiratory device, the inspiratory conduit 16, and/or cannula 17. The electronics boards 272 can be in electrical communication with those sensors. Output from the sensors can be received by the controller 13, to assist the controller 13 to operate the respiratory system 10 in a manner that provides optimal therapy, including meeting inspiratory demand when the system is a high flow therapy system.

As outlined above, the electronics boards 272 and other electrical and electronic components can be pneumatically isolated from the gases flow path to improve safety. The sealing also prevents water ingress.

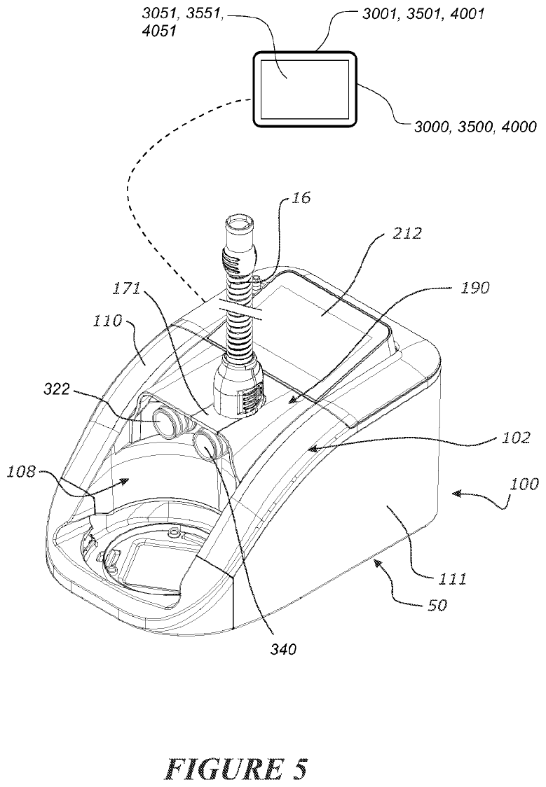

FIG. 5 shows additional details of an alternative configuration of the breathing assistance apparatus of FIGS. 4B-4D. The breathing assistance apparatus may have any one or more features described for the apparatus of FIGS. 4B-4D.

The breathing assistance apparatus 10 may have any one or more of the features and/or functionality of the breathing assistance apparatus described and shown in WO2016/207838A9 and US patents application publication no. 2018/0185606 or U.S. patent application Nos. 62/755,936, 62/890,866, 62/925,971, and PCT/IB2019/059463 (published as WO 2020/095186). The contents of those specifications are incorporated herein in their entirety by way of reference.

FIGS. 5 to 7 show details of the removable elbow 171 that embodies the humidified gas inlet port 340 and the gasflow outlet port 21. Although this section describes the features of the humidified gas inlet port 340 including a seal 173, the features of the gas outlet port 322 of the housing and its interaction with the gas inlet port 306 of the humidification chamber will be the same.

The humidified gas inlet port 340 comprises a generally horizontally oriented extended portion that is configured to insert within the gas outlet port 308 of the humidification chamber. The terminal end 340a of the port has a rounded edge to aid in aligning the gas outlet port 322 with the humidified gas inlet port 340. Additionally, the terminal end 340a is slightly smaller in diameter than the gas outlet port 308.

At least one recessed portion is provided on the port 340. This recessed portion allows a seal 173 to be attached to the port.

The humidified gas inlet port 340 may comprise a plurality of seals or sealing elements located in the recess. The plurality of seals 175 may be a pair of wiper seals, L-seals, X-rings, or O-rings. The wiper seals may have a T-shaped cross-section. In some configurations, the gas inlet port 163 may comprise three or more seals or sealing elements. A similar seal arrangement can also be on the outlet port 322 of the base unit 50. The wiper seals, i.e. double seals, prevent or reduce breathing gas leak and/or condensate from moving towards the electronics in the removable elbow 171 and the electrical connector 178 (described below) of the elbow. Similarly, the seals reduce the chance of, and preferably prevent, liquid, i.e. condensate, from moving and dripping back into the gas outlet port 322 of the base unit 50 to prevent water ingress into the electronics chamber of the base unit.

The seal 173 may be made from silicone rubber. In an alternative configuration, the seal 173 could be made from any suitable elastomer, such as polyurethane. Alternatively, the seal 173 may be made from thermoplastic elastomer(s) and/or thermoplastic vulcanisate(s), particularly if the seal will be overmoulded onto the removable elbow.

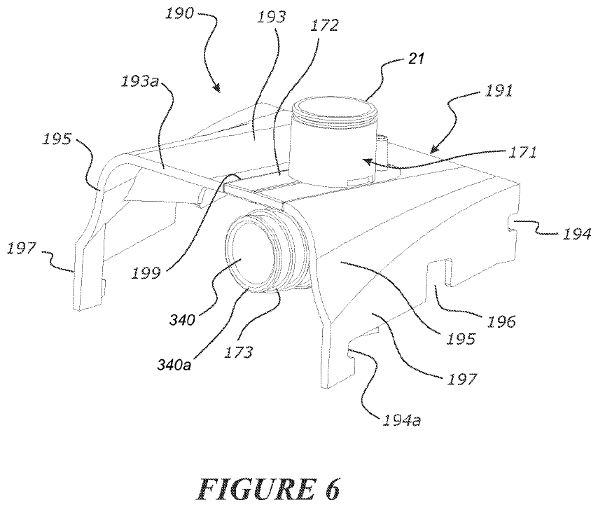

The base unit 50 of the breathing assistance apparatus 10 comprises a shroud 190 that cooperates with the housing 100 and the removable elbow 171. FIG. 6 shows the removable elbow 171 connected to the shroud 190. As shown in FIG. 5 for example, the shroud 190 serves to create a uniform upper surface of the housing 100 of the apparatus 10, with the patient outlet port 21 of the removable elbow 171 protruding upwardly through the shroud 190. The shroud 190 is configured such that it is not removable from the housing 100 in normal use of the apparatus 10.

As shown in FIG. 6, the shroud 190 comprise a body 191 having a substantially flat horizontal upper surface 193, two contoured shoulders 195 having a substantially sinuous configuration that extend downwardly and outwardly from opposite sides of the upper surface 193, and two substantially vertical downwardly extending outer side walls 197. A recess 199 extends rearwardly into the upper surface 193 from a forward edge 193a of the upper surface 193. The recess 199 is sized and configured to receive part of the removable elbow 171, to provide an unobstructed path for the removable elbow 171 to be connected to the housing 100 of the base unit 50 of the apparatus 10.

Similarly, the removable elbow 171 has a flat horizontal tab 172 extending from the elbow that has a shape that is complementary to the shape of the recess 199 in the shroud, such that when the removable elbow 171 is assembled with the apparatus 10, the flat horizontal tab 172 is received in the recess 199 to create a uniform surface. This tab 172 can additionally provide an upper surface for the conduit 16 to contact when connecting the conduit 16 to the patient outlet port 21 of the elbow.

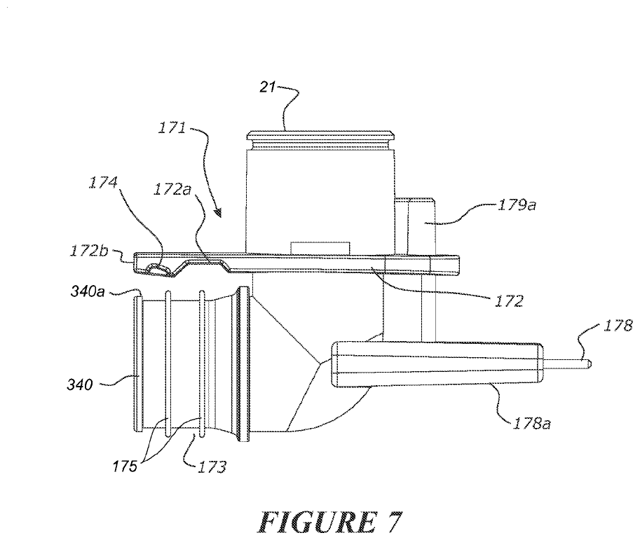

As shown in FIG. 7, the flat horizontal tab 172 can have a thinned portion 172a adjacent the terminal forward end portion 172b, located between the patient outlet port 30 of the elbow and the terminal forward end portion 172b of the tab. This allows the terminal forward end portion 172b to flex vertically relative to the rest of the elbow 171.

The flat horizontal tab 172 also has engagement features comprising two protrusions 174 extending outwardly from opposite sides of the terminal forward end portion 172b of the tab. The protrusions 174 are configured to interact with engagement features comprising complementary engagement recesses (not shown) extending outwardly from either side wall 199a of the recess 199 of the shroud of the housing, on the underside of the shroud. The removable elbow 171 is configured to connect to the housing by moving the removable elbow in a first direction relative to the housing (rearwards towards the housing). The removable elbow 171 is configured to disconnect from the housing by moving the removable elbow 171 in a second direction (forwards relative to the housing) that is opposite to the first direction. Due to the interaction of the engagement features, the removable elbow 171 is configured to inhibit movement of the removable elbow 171 in the second direction in the absence of actuating part of the removable elbow, e.g. the terminal forward end portion 172b, relative to another part of the removable elbow to flex the tab. That inhibits removal of the removable elbow 171 from the shroud 190 of the housing 100.

In order to remove the elbow 171 from the housing 100, the user would typically first press downwards on the upper surface of the terminal forward end portion 172b of the tab 172, in order to actuate that part of the tab 172 by flexing the tab and disengage the protrusions 174 from the engagement recesses. Only once the tab has been flexed can a user then pull the elbow 171 out from the housing 100. One advantage of this is that it helps prevent the elbow 171 from coming loose when the humidification chamber 300 is being removed from the apparatus by pulling the humidification chamber 300 out of the recess 108.

Temperature sensor(s) such as thermistor(s) may be provided in the removable elbow 171. The thermistors may be located in the rear vertical wall of the upstanding portion of the elbow, close to the curved transition region between the vertical and horizontal elbow portions. At this location, the thermistors are relatively shielded from the heat produced by the heater plate 140, allowing for more accurate estimations of the temperature of gases flowing through the removable elbow 171 to be made.

The elbow 171 has electrical connectors positioned in an upstanding chimney 179a, the connectors configured to provide power from a main power board of the apparatus 10 to the heater wires 16a in the conduit 16.

As mentioned above, the shroud 190 is designed to not be removed during regular use. The shroud has features that allow it to be clipped onto a screen carrier 211, which in turn is fastened to the upper chassis 102 to become part of the housing 100. The screen carrier 211 can connect to and support a display 212. In alternative configurations, the screen carrier 211 may not be provided, and the shroud 190 may clip directly to part of the housing 100, such as an upper surface or upper chassis 102 of the housing.

The shroud 190 is configured to attach to the screen carrier 211 of the housing 100 via two movements; an initial movement of the shroud in a first direction followed by a subsequent movement of the shroud in a second direction that is offset from the first direction. In one configuration, the second direction is transverse to the first direction. In the form shown, the shroud 190 is configured to initially be moved in a first downward direction, followed by a movement in a second rearward direction, relative to the screen carrier 211 and thereby the housing 100. In the form shown, the downward direction DD is vertical and the rearward direction RD is horizontal.

The shroud 190 is configured so that the shroud cannot be detached from the screen carrier 211 of the housing solely by pulling the removable elbow 171 in the second forwards direction relative to the housing 100.

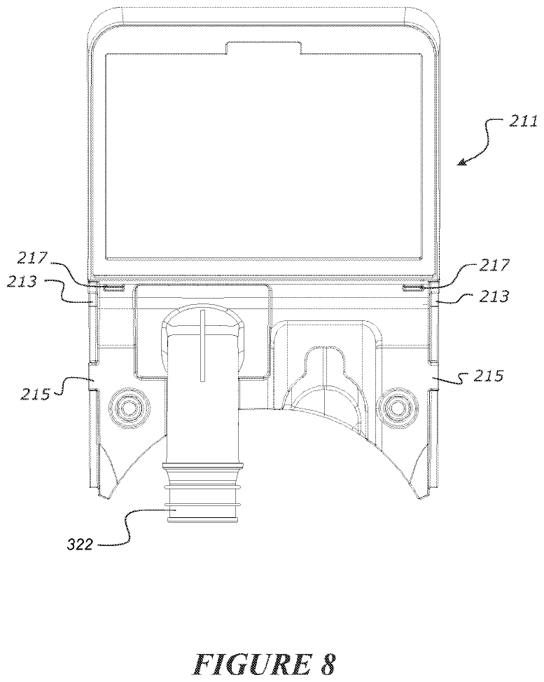

Each side 197 of the shroud 190 is shaped to be complementary to the shape of the screen carrier 211. The sides of the screen carrier 211 of the housing 100 have two forwardly directed horizontal protrusions 213 (one per side wall) that engage with complementary rearwardly open recesses 194 in rear walls on each side of the shroud 190 as the shroud is moved in the rearward direction relative to the housing (see FIG. 8). Once the shroud 190 is connected to the screen carrier 211, the horizontal protrusions 213 being received in the recesses 194 prevent vertical movement of the shroud 190.

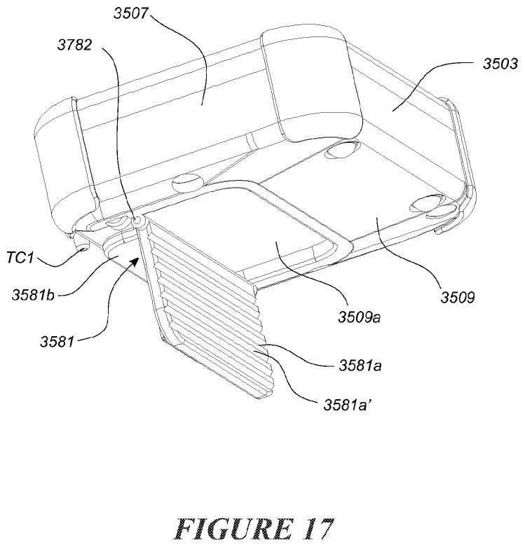

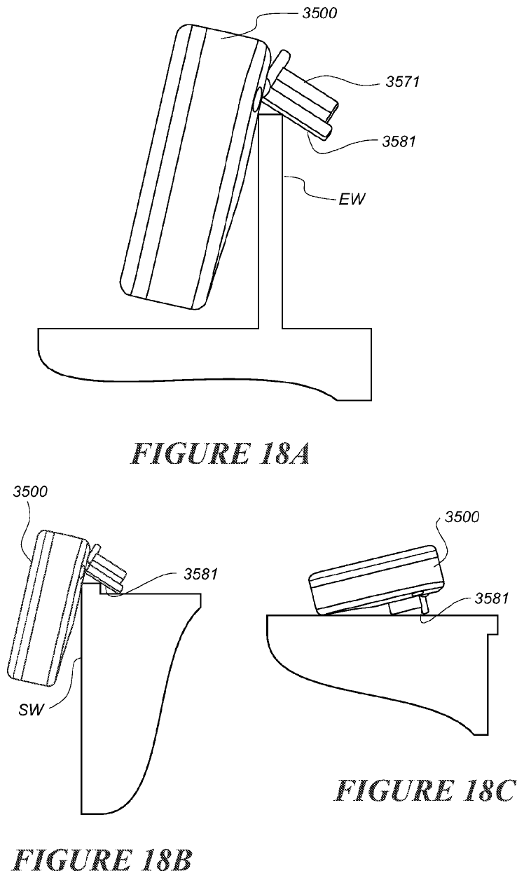

Similarly, the screen carrier 211 of the housing 100 has an upstanding vertical protrusion 215 on each side that engages with a complementary downwardly open recess 196 in the bottom of each side wall 197 of the shroud as the shroud is moved in the downward direction relative to the housing. Unlike the horizontal protrusions, the vertical protrusion 215 is narrower than the complementary recess 196 in the shroud. This allows for a small amount of horizontal movement in the rearward direction as shown in FIGS. 17 and 18. The vertical protrusion 215 and recess 196 aid in aligning the shroud 190 with the screen carrier 211 during assembly.

The shroud 190 is first placed above the screen carrier 211 and moved in the downward direction. The shroud is then slid horizontally in the rearward direction such that the horizontal protrusions 213 engage with the complementary recesses 194 in the shroud.

The shroud 190 and screen carrier 211 may have a second set of forwardly extending horizontal protrusions and rearwardly open recesses toward a forward end of the screen carrier 211 and shroud 190, to further inhibit vertical movement of the shroud 190 relative to the screen carrier 211.

The shroud 190 and screen carrier 211 of the housing 100 additionally have features to inhibit horizontal movement of the shroud relative to the screen carrier when they are fully engaged, to therefore inhibit removal of the shroud from the housing. The shroud may have one or more downwardly extending engagement protrusions (not shown) that extend downwards from the back of the lower surface of the shroud 190. The engagement protrusions are configured to engage with complementary upwardly extending engagement protrusion(s) 217 extending from an upper surface of the screen carrier 211.

The shroud can still be removed by moving the shroud 190 forwards in direction opposite to the rearward direction RD, if a large enough force is applied. However, this would only be done by a technician if some form of maintenance were being performed on the apparatus. The features above are designed such that the shroud 190 would not be removed during normal use (even accidentally).

It can be seen from FIG. 8 that the engagement features on the shroud 190 and screen carrier 211 are such that the upper surface of the screen carrier can be configured so that there are no, or a minimal number of, exposed fasteners (such as screws) on the underside of the shroud and the upper surface of the screen carrier, meaning that the shroud and upper surface of the screen carrier are easy to clean.

The removable elbow 171 is removable from the housing 100 when the shroud 100 is attached to the housing.

Referring to FIG. 7, the removable elbow 171 also includes a printed circuit board (PCB) electrical connector 178 for electrically connecting to the base unit 50 of the breathing assistance apparatus 10 to form an electrical connection with an electrical component in the main housing 100 of the base unit. The electrical connection provides an electrical link between the base unit 50 of the apparatus 10 and the temperature sensors embedded in the elbow, as well as between the base unit 50 of the apparatus 10 and the conduit 16 (when the conduit has one or more sensors and/or a heating element), via an electrical interconnecting assembly in the main housing of the base unit. The PCB electrical connector 178 electrically connects to the electrical interconnecting assembly when the removable elbow 171 is connected to the housing 100.

The PCB electrical connector 178 facilitates electrical communication of signals from the sensor in or along the conduit and/or patient interface (e.g. a patient end temperature sensor) to the controller via the main circuit board 272. The connector 178 also facilitates electrical communication of the heater wires by the controller if present in the conduit. Similarly, other sensors within the breathing assistance apparatus e.g. temperature and/or flow sensors in the elbow, are in electrical communication with the controller via the PCB electrical connector 178 and main circuit board 272.

The breathing assistance apparatus may have a controller that receives sensor readings, processes sensor readings, and/or performs other various functions e.g. calculating respiratory rate or determining oxygen concentration, controlling components of the breathing assistance apparatus, e.g. controlling operation of a blower or heater plate of the humidifier or heater wire in the conduit.

The breathing assistance apparatus may comprise an additional controller that is configured to provide control signals to components based on control signals from a main controller of the breathing assistance apparatus. For example, the components may comprise a motor of the blower, the display or portable display, and/or one or more other components.

In the configuration shown, the PCB electrical connector 178 is partly housed in a housing 178a that is integrally formed with the elbow. The PCB electrical connector projects rearwardly from the housing 178a to insert into an electrical connector on the base unit 50 of the apparatus 10 in a horizontal direction (i.e. the same rearward insertion direction in which the humidification chamber 300 connects to the housing 100 of the base unit 50 and the same rearward direction RD in which the shroud 190 connects to the screen carrier 211). The electrical connector may be part of, or in electrical communication with, an interconnect circuit board 271 of the base unit 50, which is described below.

A portable display unit 3000, 3500, 4000 may be provided for use with, or in combination with, any of the breathing assistance apparatuses described herein.

The specification discloses several configurations of a portable display unit 3000, 3500, 4000. Any one or more of the described features and functionality can be used in various combinations in any one more of the portable display units.

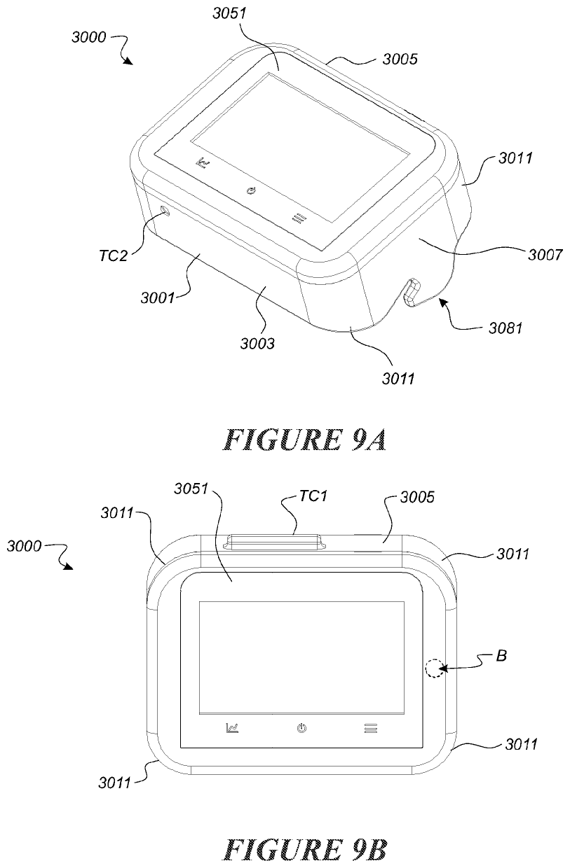

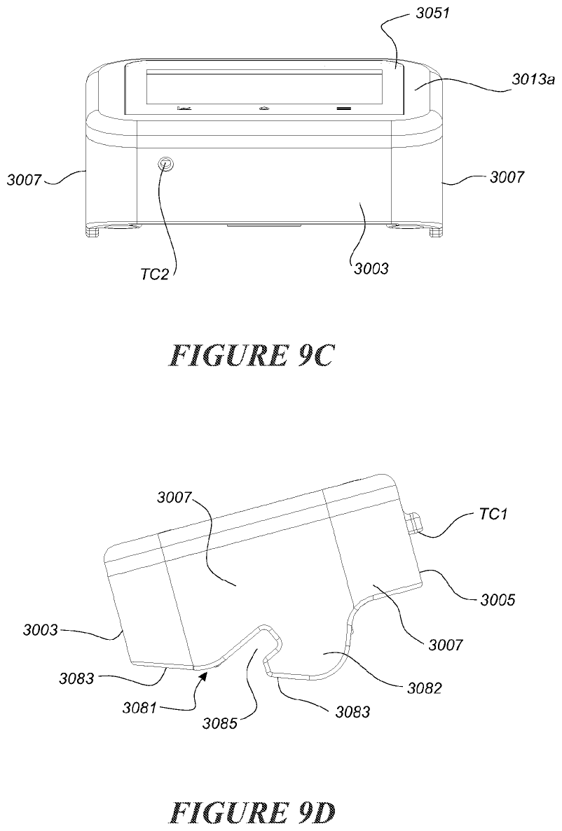

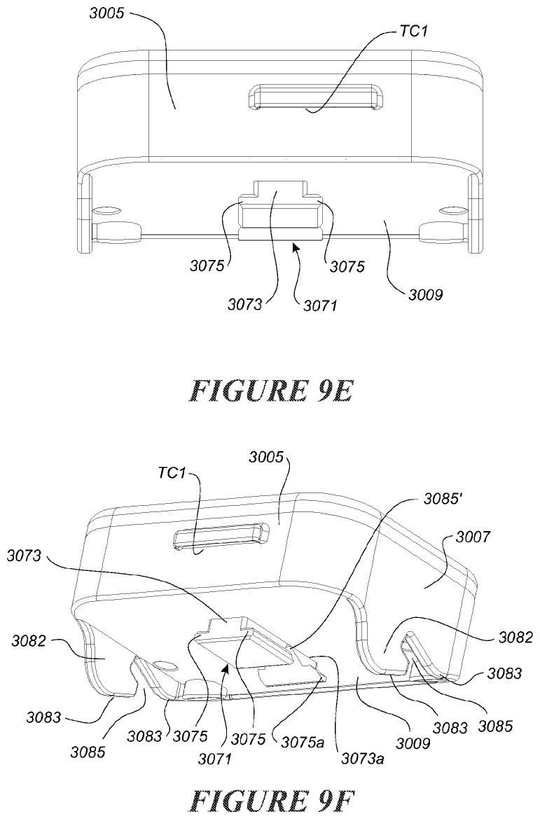

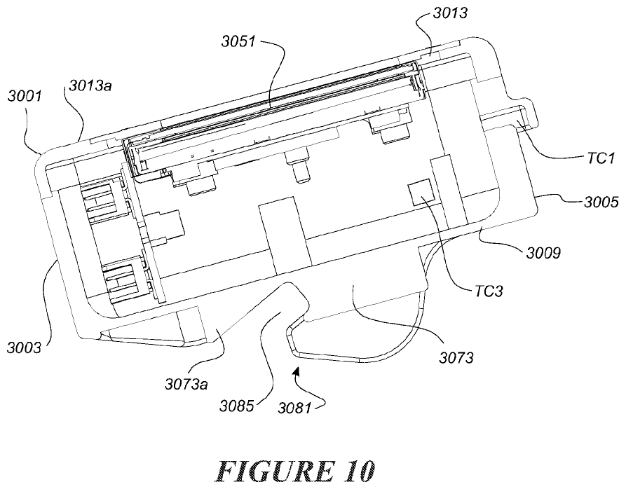

FIGS. 9A to 10 show a first configuration of the portable display unit 3000. The portable display unit 3000 comprises a display unit housing 3001, a screen 3051, and a tethering component (TC1, TC2, and/or TC3) for tethering the portable display unit 3000 with a base unit 50 of the breathing assistance apparatus.

The screen 3051 may be a touch screen to enable the user to interact with the screen to control one or more functions of the breathing assistance apparatus in addition to the screen 3051 displaying information.

The screen 3051 may be a capacitive touch screen. Alternatively, the screen 3051 may be a resistive touch screen. A resistive touch screen may provide improved performance in environments where the screen may be exposed to liquids.

Alternatively, the screen may not be a touch screen and may only be used for display purposes.

In addition to, or alternatively to, a touch screen, the portable display unit 3000 could comprise user input component(s) such as button(s) or dial(s). Examples are described below.

The display unit housing 3001 comprises a front wall 3003, a rear wall 3005, opposed side walls 3007, and a base 3009.

Curved surfaces 3011 provide smooth intersections between the front, side, and front and rear walls.

An upper surface 3013 of the display unit housing 3001 forms a screen support for receipt of the screen 3051. A portion of the screen support is recessed so the screen 3051 can sit flush with the uppermost surface 3013a of the housing. In some configurations, the screen support may be configured so that the screen 3051 is spaced rearwardly from the uppermost surface 3013a of the housing, to provide protection for the screen.

The interior of the display unit housing 3001 may be substantially hollow to provide space for housing or routing electrical and/or electronics components.

In the form shown the display unit housing 3001 has a substantially rectangular shape in plan view. It will be appreciated that the display unit housing 3001 could have any other suitable shape such as square, polymeric, elliptical, or round for example.

The tethering component TC1, TC2 may be a component that is configured for physically tethering the portable display unit 3000 with the base unit of the breathing assistance apparatus.

By physically tethering the portable display unit 3000 with the breathing assistance apparatus, the portable display unit 3000 may be moved relative to the breathing assistance apparatus but only within a limited distance. That may be of benefit to minimise the likelihood of the portable display unit 3000 being misplaced, particularly in an environment where multiple breathing assistance apparatuses and multiple display units are used.

The tethering component may comprise a cable 3061. The cable may be any suitable type of elongate flexible member including, but not limited to, a cord, a metal, polymeric, or combination cable, a rope, or a string.

The cable may be resilient and can be stretched and returned to its original unloaded length. That provides flexibility, the ability to move the portable display unit 3000 to a desired use location, and reduces load transfer on the portable display unit due to movement of the base unit 50.

The cable may be integrally formed with or coupled to the portable display unit. The cable may be separable from the portable display unit 3000 or may be permanently affixed to the portable display unit 3000.

This connection may be captive and designed only to be removed during servicing of the apparatus. For example, there may be a retaining feature that is removable by a technician by screws or otherwise, but the cable is not removable during normal use.

If the cable is separable from, or provided separately from, the portable display unit 3000, the tethering component TC1, TC2 may comprise a connector for connecting the cable 3061 to the portable display unit 3000.

Similarly, if the cable is separable from, or provided separately from, the base unit 50, the base unit 50 may a connector for connecting the cable 3061 to the base unit.

In the configuration shown, the housing 3001 of the portable display unit 3000 comprises a first tethering component TC1 comprising a first connector port for connecting to the cable 3061. The housing 3001 of the portable display unit 3000 may comprise an optional second tethering component TC2 comprising a second connector port for connecting to an optional second cable (not shown).

In the configuration shown, the first tethering component TC1 is provided in the rear wall 3005 of the housing 3001. By providing the first tethering component TC1 in the rear wall 3005, the cable 3061 is unlikely to interfere with the user's interaction with the screen 3051 or other components of the breathing assistance apparatus. However, the tethering component(s) TC1, TC2 can be provided in any suitable surface(s) of the housing 3001.

The cable 3061 may be resiliently flexible and/or may be retractable into the display unit housing 3001 or the base unit 50 of the breathing assistance apparatus. That enables the cable 3061 to lengthen as required during movement and positing of the portable display unit 3000, but to return substantially to an initial length when force is removed from the cable 3061.

In one configuration, the cable 3061 comprises a coiled arrangement that lengthens upon application of a tensile force. In another configuration, the cable 3061 comprises a length of elastomeric material that lengthens upon application of a tensile force.

Additionally or alternatively, the display unit housing 3001 or the base unit 50 may contain a retracting mechanism for retracting the cable 3061 when tensile force is removed. For example, the retracting mechanism may comprise a biased reel that the cable 3061 is wound around. The biased reel may enable a user to pull the cable 3061 off the reel to move the portable display unit 3000, and may lock the position of the reel. The user could then pull the cable to release the lock, which will cause the biased reel to retract the cable 3061 as the user moves the portable display unit 3000 to a position closer to the base unit 50 of the breathing assistance apparatus.

In some exemplary configurations, the cable length may be between about 0.5 m to 3 m, optionally between about 1.5 m and about 2.5 m. The cable length may be up to about 2 m. The cable length may be between about 1.5 m and about 2 m.

In one example, the length of the cable may be the same length or longer than the inspiratory conduit. In some configurations, the cable is longer than the inspiratory conduit. In some configurations, the cable is as long as or longer than the inspiratory conduit and the interface conduit. This allows the display unit to be moved freely. For example the display unit can be disconnected from the base unit and positioned at a location that the display unit is easy to view for example near the patient. This allows the clinician to get information from the display unit while still keeping the base unit away from the patient.

The cable 3061 may be able to be disconnected from the portable display unit 3000 or from the base unit 50.

The cable 3061 may solely provide a physical tether between the portable display unit 3000 and the base unit of the breathing assistance apparatus. Alternatively, the cable may provide electrical or electronic communication between the portable display unit and the base unit.

The cable may communicate power to the portable display unit from the base unit and/or may communicate data between the portable display unit and the base unit, both when the portable display unit is connected to the base unit and when the portable display unit is separated from the base unit. Therefore, the cable may comprise at least one wire for communicating power to the portable display unit from the base unit and/or for communicating data between the portable display unit and the base unit.

When the cable communicates power and/or data and/or control signals, the length of the cable may be selected to minimise latency. In one exemplary configuration, the cable may have a similar length to the inspiratory conduit of the breathing circuit. For example, a length of about 1.5 m. This may be useful for assisting with enabling various therapy modes.

The tethering component connector(s) TC1, TC2 on the portable display unit 3000 may comprise an electrical connector that is in communication with component(s) of the portable display unit to communicate power and/or data between the base unit and the portable display unit. The cable 3061 may communicate both data and/or power. Alternatively, the cable may communicate one of data and power, and another cable coupled to TC2 may communicate the other one of data and power.

One or both ends of the cable 3061 may comprise an electrical connector for connecting the wire(s) of the cable to respective electrical ports on the base unit 50 and portable display unit 3000.

In an alternative configuration in which the cable 3061 solely provides a physical tether between the portable display unit 3000 and the base unit 50 of the breathing assistance apparatus, a separate cable may provide power and/or data communication between the base unit and the portable display unit 3000.

The cable 3061 can be disconnected from the base unit 50 to allow the portable display unit 3000 to be completely detached from the base unit 50.

In a further alternative configuration, the cable 3061 may be co-axial with the inspiratory conduit. In a further alternative configuration, the cable may be wrapped around the inspiratory conduit in order to stow the cable when the portable display unit 3000 is disconnected. The cable being wrapped around the inspiratory conduit can tidily stow the cable and reduce the chances of free cables.

Additionally or alternatively to the tethering components TC1, TC2, the tethering component TC3 may comprise a component of a wireless communication interface for communicating control signals and/or other data between the portable display unit 3000 and the base unit.

The wireless component may comprise a wireless data transmitter and/or receiver or a transceiver to communicate with a wireless data transmitter and/or receiver of the breathing assistance apparatus to communicate data between the base unit and the portable display unit 3000.

The wireless communication may be of any suitable configuration such as, but not limited to, WIFI, near field communication (NFC), Bluetooth.

The portable display unit 3000 may use a combination of physical and wireless tethering with the base unit of the breathing assistance apparatus. For example, the portable display unit 3000 may be physically tethered by the cable 3061 and optionally may be powered by the cable 3061, but may be wirelessly tethered with the base unit for data communication. In another alternative, there may be no physical tether between the portable display unit 3000 and the base unit, but the portable display unit 3000 may be wirelessly tethered with the base unit. In that configuration, the portable display unit 3000 may be battery powered. The battery may be rechargeable, and may be powered by a removable cable, by electrically coupling the portable display unit with the base unit, or by induction charging when the portable display unit is connected to, or brought into contact with, the base unit.

The portable display unit may couple to a docking port on the base unit to provide an electrical connection between the portable display unit and base unit to charge the battery of the portable display unit.

In some configurations, the portable display unit may comprise a battery to power the portable display unit. The breathing assistance apparatus may comprise a wireless power transfer system integrated into the breathing assistance apparatus. The breathing assistance apparatus may comprise an inductive power transfer system that can be used to charge the battery of the portable display unit. For example, the base unit may comprise a charging coil and the portable display unit comprises a receiving coil. The charging coil can inductively couple to the receiving coil and induce current in the receiving coil. The battery is electrically connected to the receiving coil and can be charged by the induced current. The charging coil is supplied current by the controller of the base unit.

The battery of the portable display unit 3000 may function as a backup power source if the cable 3061 is disconnected or malfunctions, or may in some embodiments (such as when the portable display unit 3000 has wireless functionality to the base unit 50 or other medical devices) be the main power source for the portable display unit 3000.

The battery may be rechargeable. In some configurations, the battery may be charged via a charging port. The charging port may align with the tethering connector TC1, TC2 of the portable display unit.

The portable display unit 3000 may be a stand-alone unit that remains physically separate from the base unit (other than a physical tether, if any). Alternatively, the portable display unit 3000 may be physically connectable to the base unit 50. The portable display unit 3000 may be removable or separable from the base unit 50 to enable the portable display unit 3000 to be positioned in a desired remote location for use.

When the portable display unit 3000 is removably connectable to the base unit 50, the portable display unit 3000 comprises at least one connection feature 3071 for removably connecting the portable display unit 3000 to the base unit and optionally to a different support component.

The at least one connection feature 3071 may be arranged to interact with complementary connection feature(s) on the base unit and/or on the different support component.

The at least one connection feature 3071 may comprise any one or more suitable features to enable removable connection of the portable display unit 3000 with the base unit 50 and/or the different support component. Exemplary suitable connection features include, but are not limited to, adhesives, hook and loop fasteners, fasteners, threaded connections, clips, and clamps, protrusions or recesses. An alternative connection feature comprises a latch mechanism. For example, the portable display unit 3000 may comprise a pair of latches that can be used to couple to corresponding receiving features on the base unit 50 and/or on the different support component.

An additional or alternative connection feature 3071 may comprise magnet(s) and may predominantly use magnetic force for the connection or may use magnetic force to bias the connection.

For example, one of the portable display unit 3000 and the base unit 50/different support component may comprise one or more magnets, and the other of the portable display unit 3000 and the base unit 50/different support component may comprise one or more magnets or magnetisable material. The magnet(s) on the portable display unit 3000 or base unit 50/different support component may cooperate with the magnet(s) or magnetisable material on the other of the portable display unit 3000 and base unit 50/different support component.

The connection feature(s) may provide haptic or audible feedback for connection.

The connection feature 3071 on the portable display unit 3000 may comprise a first component of a sliding connector. The first component may be engageable with a second component of the sliding connector on the base unit 50 or on a different support component.

The first component and second component may be engageable with each other in a sliding action of one component relative to the other. For example, the first component may be engageable with the second component in a sliding action in a first direction of one component relative to the other.

Once engaged, the first component and second component may not be disengageable by attempted movement of the first component relative to the second component in a direction transverse to the first direction. The first component may only be disengageable from the second component by moving the first component relative to the second component in a second direction that is opposite to the first direction.

One of the first component and the second component may comprise a male component, and the other of the first component and the second component may comprise a complementary female component to receive the male component. For example, the male component may be provided on the portable display unit 3000 and the female component may be provided on the base unit 50 or on the different support component. Alternatively, the female component may be provided on the portable display unit 3000 and the male component may be provided on the base unit 50 or on the different support component.

In some configurations, the male component may comprise a tongue and the female component may comprise a receiver or cradle for the tongue.

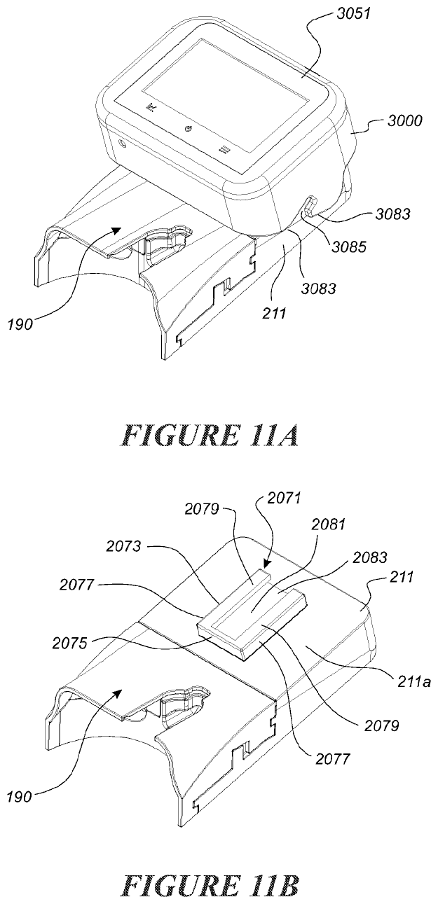

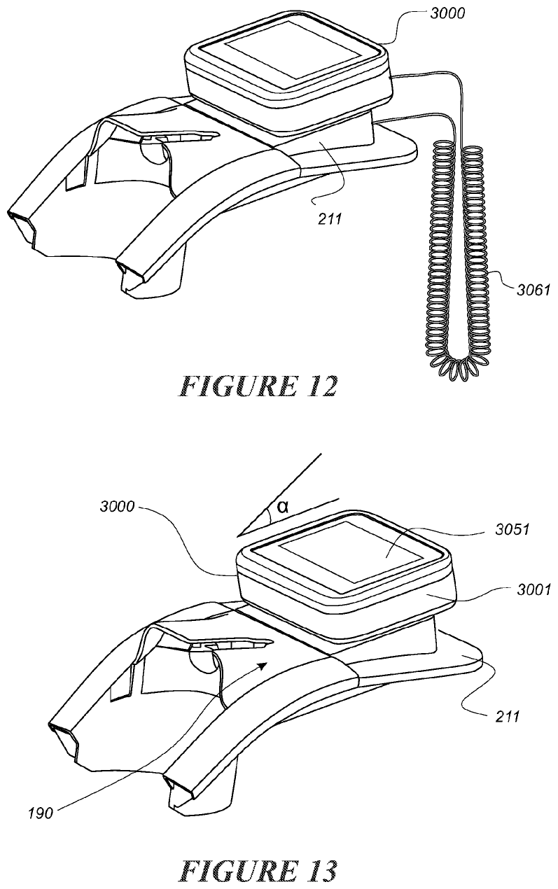

FIGS. 9E and 9F show an exemplary connection feature 3071 on the base 3009 of the display unit housing 3001 for engaging with a complementary connection feature 2071 on the base unit (FIG. 11B). The same or a similar complementary connection feature 2071 could be used on the different support component.

In the configuration shown, the complementary connection feature 2071 is provided on an upper angled surface 211a of the screen carrier 211 of the base unit.

The complementary connection feature comprises a female component.

In the configuration shown, the complementary connection feature comprises a connection housing 2073 which acts as a receiver.

The connection housing comprises a forward wall 2075, two side walls 2077, and two inwardly directed flanges 2079. The flanges 2079 are spaced apart to provide a relatively narrow spacing 2081 between the flanges. An end of the housing opposite the forward wall 2075 defines a relatively wide opening 2083 that is wider than the spacing 2081.

The connection feature 3071 comprises a male component. In the form shown, the male component comprises a tongue but could alternatively have different configurations.

The tongue comprises a relatively narrow elongate body member 3073 that extends downwardly from the base 3009 of the display unit housing 3001. The body member 3073 has a dimension that is slightly narrower than the spacing 2081. Flanges 3075 project outwardly from either side of the bottom of the body member 3073, and have a dimension that is slightly narrower than the opening 2083.

The connection feature 3071 may comprise a single elongate member, or may comprise a plurality of members. In the configuration shown, the connection feature 3071 comprises an elongate member that provides a rear portion of features 3073, 3075, and a spaced part separate forward member that provides a forward portion 3073a, 3075b of those members.

The portable display unit 3000 connects to, and separates from, the housing of the base unit 50 by a sliding action of the portable display unit relative to the base unit. In the configuration shown, the flanges 3075, 3075a are inserted into the connection housing under the flanges 2075 and slid forwardly. Because the upper surface 211a of the screen carrier is angled, gravity will assist with retaining the portable display unit 3000 in connection with the base unit 50. To separate the portable display unit 3000 from the base unit, the portable display unit is slid rearwardly relative to the screen carrier 211 until the flanges 3075 clear the flanges 2075.

Rather than being provided on the base 3009 of the portable display unit 3000 and the upper surface 211a of the screen carrier 211, the connection features 3071, 2071 could be provided elsewhere on the portable display unit 3000 and/or base unit 50, with the connection and removal directions varying accordingly.

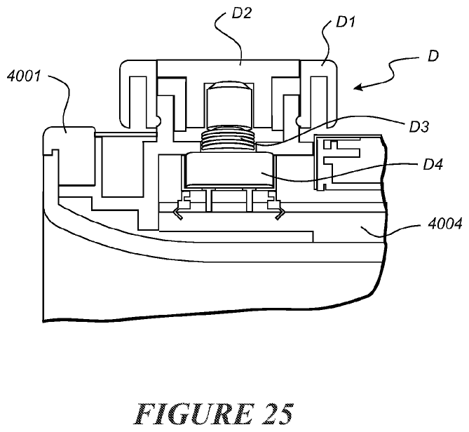

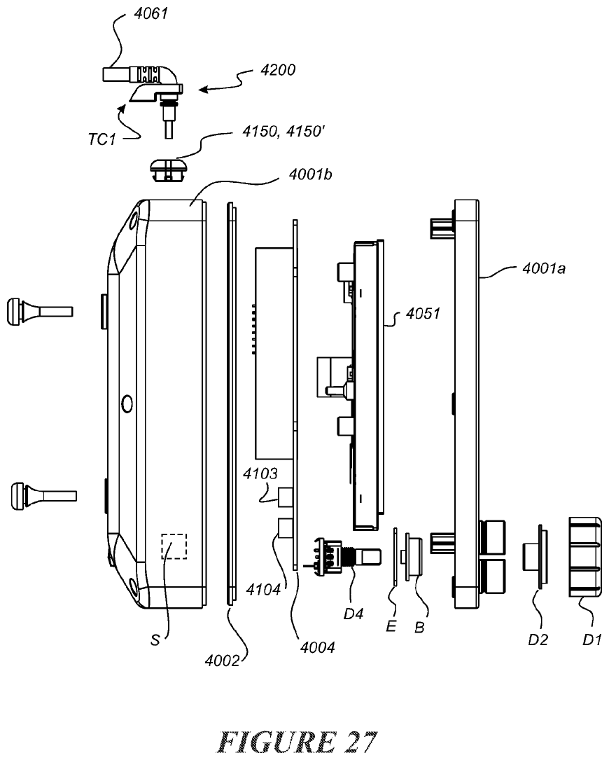

The portable display unit 3000 comprises a circuit board with appropriate electrical components. Although not shown in this configuration, a configuration of the portable display unit 4000 with the circuit board 4004 is shown in FIGS. 25 and 27.

The circuit board may comprise a printed circuit board (PCB).

The circuit board of the portable display unit 3000 comprises a controller such as a microcontroller or microprocessor that controls the operations of the display unit, processes signals received from the base unit (i.e. from the base unit microcontroller or microprocessor), and generate control signals for example in response to user input via the screen 3051 or other user input component(s) such as button(s) or dial(s). The circuit board/controller may receive data relating to respiratory parameters or other usage data from the base unit. The circuit board/controller may send control signals to the base unit. The portable display unit 3000 may further comprise a communication unit e.g. a communication chip that provides wireless communication capabilities for the portable display unit. Further the base unit also comprises a communication unit that allows the base unit to wirelessly communicate with the portable display unit.

The portable display unit comprises a communication interface for communicating data and/or control signals between the base unit and the portable display unit, both when the portable display unit is connected to the base unit and when the portable display unit is separated from the base unit. The communication interface may be provided by any of the options outlined above, e.g. the cable 3061, a different wire, and/or wireless communication.

The circuit board may be in electrical and/or electronic communication with the screen 3051, a backlight, and one or more user input components such as button(s) or dial(s). The circuit board may send power and data signals to and/or from the screen 3051, backlight, and user input components(s).

The portable display unit 3000 may optionally comprise at least one support feature for supporting the portable display unit on a support surface (that is different from the base unit 50).

The support feature(s) may be configured to support the portable display unit 3000 on one support surface, or may be configured to support the portable display unit 3000 on a plurality of different support surfaces.

The support feature(s) may be fixed relative to the housing 3001 or may be adjustable relative to the housing 3001 (e.g. one or more pivoting legs or supports).

In the configuration shown, the portable display unit 3000 comprises a support feature 3081 for supporting the portable display unit on a plurality of different support surfaces.

The support feature 3081 is arranged to support the portable display unit 3000 on a substantially horizontal support surface or on a medical pole.

In the configuration shown, the support feature comprises a projecting wall 3082 that provides a base edge 3083 for resting on a substantially horizontal support surface. The projecting wall 3082 extends downwardly from each side wall 3007 of the display unit housing, beyond the base 3009 of the base unit housing.

The base edge 3083 is at an acute angle relative to the upper surface 3013 of the housing, so that the screen is inclined toward a user when the base edge 3083 is on the support surface.

Each projecting wall 3082 optionally comprises an angled slot 3085 that extends upwardly and rearwardly from the base edge 3083 to form a spacing between forward and rearward portions of the base edge. The angled slot 3085 terminates at or adjacent the base 3009 of the housing 3001.

The slot(s) 3085 act as hanging hook(s) and enable(s) the portable display unit 3000 to be supported on a horizontally extending stud or arm extending outwardly from a medical pole for example, with the stud or arm extending through the angled slot(s) 3085.

Because the angled slots 3085 are aligned with the connection feature 3071, the connection feature 3071 may have a corresponding angled slot 3085′ between the forward 3073a, 3075a and rearward 3073, 3075 portions of the connection feature 3071 (see FIG. 9F). That may be provided in addition to, or instead of, the projecting walls 3082 and angled slots 3085. The support feature comprising the angled slot 3085′ is provided by the same structural element as the connection feature 3071. The angled slot 3085′ and the connection feature 3071 may be injection moulded as part of the same polymeric component for example.

The projecting walls 3083 provide features that enable a user to grip the unit 3000 and/or tilt the unit 3000.

As shown schematically in FIG. 13, in some configurations an angular orientation a of the screen 3051 can be adjusted relative to the display unit housing 3001. Alternatively, in some configurations an angular orientation of the display unit housing 3001 can be adjusted relative to the at least one connection feature 3071 and/or the at least one support feature 3081.

In some configurations, the screen 3051 may be rotatable around an axis that extends orthogonally through the screen 3051. The rotation may be of the screen 3051 relative to the display unit housing 3001. Alternatively, the rotation may be provided by the display unit housing 3001 being rotatable relative to the at least one connection feature 3071 and/or the at least one support feature 3081. Alternatively, the rotation may be provided between the at least one connection feature 3071 and the complementary connection feature 2071.

Accordingly, the screen 3051 may be angularly adjusted and/or rotated to an optimal viewing angle relative to the base unit 50 and/or a different support component and/or support surface.

A pivot structure may be provided between the screen 3051 and the display unit housing 3001, or between the display unit housing 3001 and the at least one connection feature 3071 and/or the at least one support feature 3081.

The pivot structure may comprise a frictional arrangement, an indexing arrangement, a locking pin arrangement, or a ratchet arrangement to enable the screen 3051 or display unit housing 3001 to be locked in a plurality of different angular positions.

The various configurations will enable an angular orientation of the screen 3051 to be adjusted to an optimal angle relative to the base unit 50 of the breathing assistance apparatus, relative to a different support component, or relative to a support surface that the portable display unit is being supported by.

By way of example, the angle of the screen may be between about 0 and about 90 degrees relative to a horizontal plane. In some configurations, the angle is between about 10 degrees and about 75 degrees. In some configurations, the angle may be between about 15 degrees and about 60 degrees.

In some configurations, the screen may be at an angle of between about 10 degrees and about 20 degrees relative to a horizontal plane, and optionally about 15 degrees relative to a horizontal plane, when the portable display unit is engaged to the base unit 50.

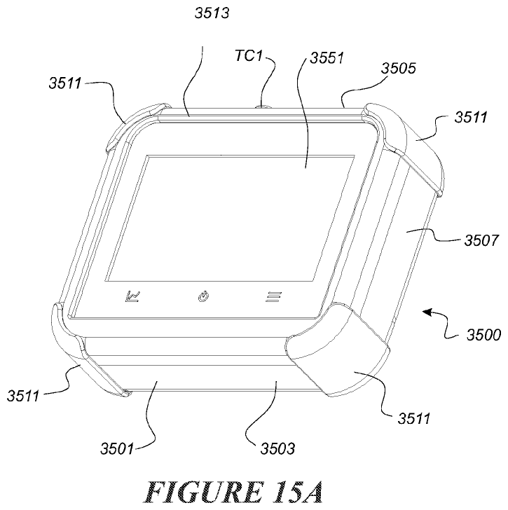

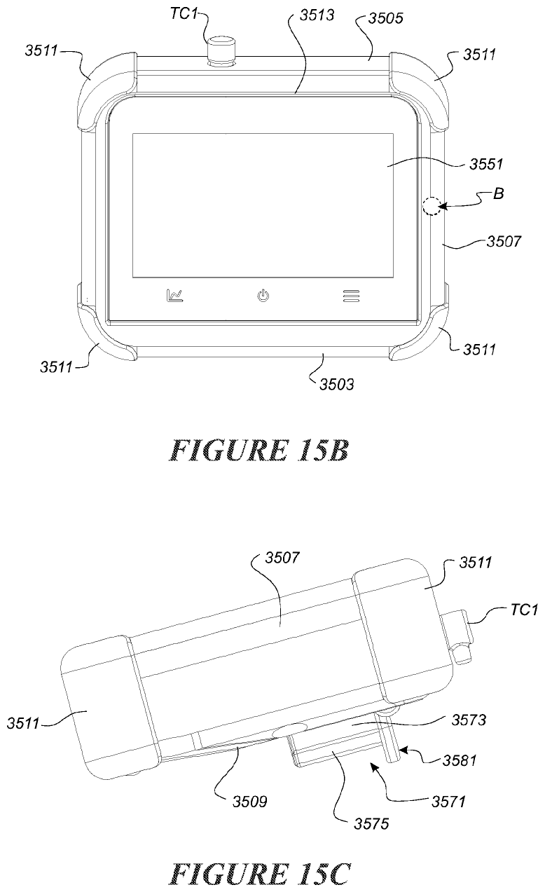

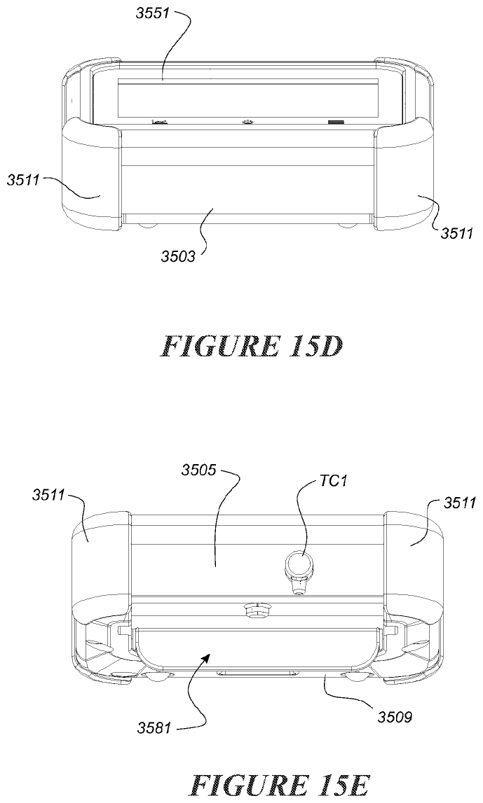

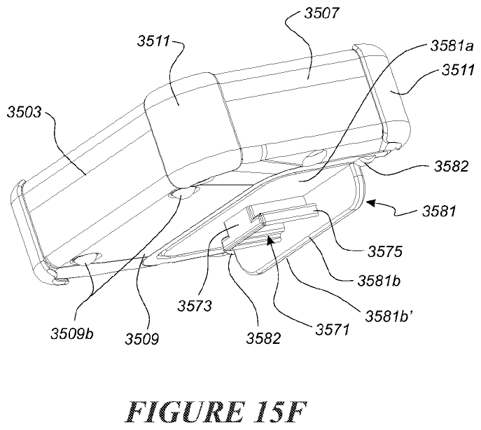

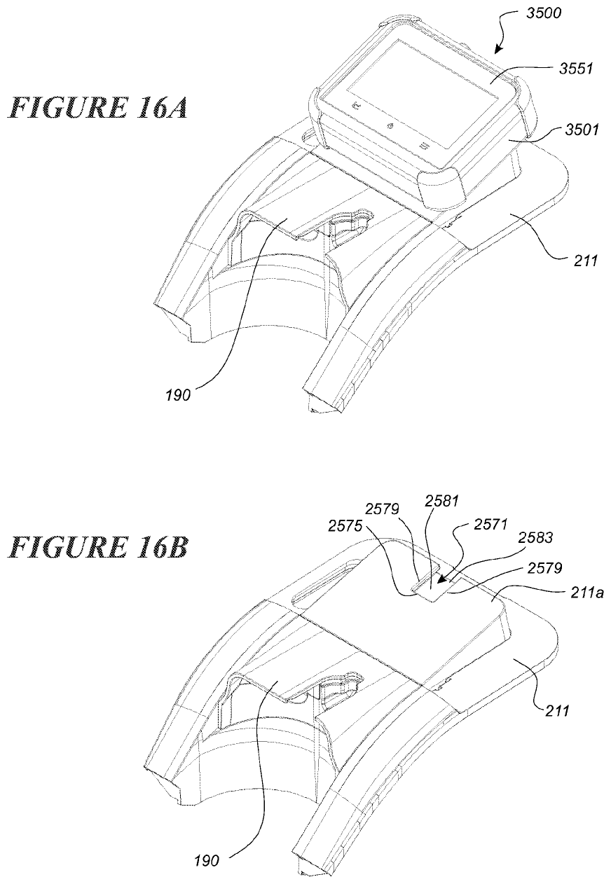

FIGS. 15A-15F show an alternative portable display unit 3500 for use with any of the breathing assistance apparatuses described herein. Unless described differently below, the features, functioning, and options are the same as for portable display unit 3000, and like reference numerals indicate like parts with the addition of 500.

As outlined above, any one or more described features and functionality can be used in various combinations in any one more of the portable display units 3000, 3500, 4000.

In this configuration, the screen 3551 is further recessed from the upper surface 3513 of the display unit housing 3501. That is, the display unit housing 3501 projects upwardly more than the screen 3551. That provides a level of drop or impact protection for the screen—if the portable display unit 3500 is dropped or experiences an impact, there is unlikely to be a direct impact on the screen 3551.

The corners of the display unit housing 3501 between the walls 3503, 3507, 3509 are covered by an impact absorbent relatively soft material 3511. The impact absorbent material 3511 may extend partly over the upper surface 3513 and base 3509. Covering the corners of the display unit housing 3501 with impact absorbent material reduces the likelihood of damage to the housing 3501 if the portable display unit 3500 is dropped or experiences an impact. The material 3511 may be integrally formed with the display unit housing 3501 or may be formed separately from, then attached to, the display unit housing 3501 The material 3511 may be removable from the display unit housing 3501.

Material selection may involve semi-rigid material which is tough enough to withstand impact. That is, the display unit housing 3501 or at least the corner sections of the display unit housing 3501 may comprise a semi-rigid material which is tough enough to withstand impact. This may be a singular piece with increased thickness at the corners 3511 of the portable display unit 3500. The material will advantageously be thin enough, and transparent, to enable actuation/use of the touchscreen.

Exemplary materials may include silicone or polyurethane. Other foam materials may be used to provide impact absorption.

In the configuration shown, the tethering component TC1 comprises a port that projects from the housing 3501 for connection to the cable 3061.

The connection feature 3571 and the support feature 3581 are provided by the same structural element.

The connection feature 3571 is substantially the same as the connection feature 3071; however, rather than being provided on the base 3059 of the housing 3501, it is provided on the same structural element that provides the support feature 3581.

The structural element that provides the support feature 3581 comprises a component that is pivotally mounted to the housing 3051 by pivot structure 3582.

In the form shown in FIGS. 15F and 17, the structural element comprises a generally L-shaped member having a relatively large body 3581a and a relatively small extension 3581b. The base 3509 of the housing 3501 comprises a recess 3509a to receive the relatively large body when the portable display unit 3500 is in the configuration shown in FIG. 15F for connecting the portable display unit 3500 to the base unit 50 of the breathing assistance apparatus.

The complementary connection feature 2571 on the base unit is substantially the same as connection feature 2071, except that the connection feature 2571 is recessed so as to not project upwardly beyond the upper surface 211a of the screen carrier 211.

When the portable display unit 3500 is separated from the breathing assistance apparatus, it can be supported on a substantially horizontal support surface on the bottom edge 3581b′ of the extension 3581b and on forward feet 3509b of the housing 3501 (FIG. 15F).

When a user wants to hang the portable display unit 3500 on a support surface, they can pivot the structural element from the position shown in FIG. 15F towards the position shown in FIG. 17, to hang the portable display unit 3500 as shown in FIGS. 18A and 18B for example.

The structural element may be pivotable through any angle up to 90 degrees, but in some configurations may be pivotable through smaller angles, such as up to 80 degrees, up to 70 degrees, up to 60 degrees, up to 50 degrees, up to 40 degrees, or up to 30 degrees for example.

The pivot structure 3582 may comprise a frictional arrangement, an indexing arrangement, a locking pin arrangement, or a ratchet arrangement to enable the structural element to be locked in a plurality of different angular positions.

As shown in FIG. 17, the relatively large body 3581a may comprise a rough surface 3581a′ to assist with gripping on to a support surface when the portable display unit 3500 is hanging. The rough surface may comprise a plurality of protrusions, ribs, ridges, channels, bumps, or the like. In the form shown, the rough surface 3581a′ comprises a plurality of transversely extending ribs.

The rough surfaces is provided on the opposite side of the relatively large body 3581a to the connection feature 3571, and is positioned in the recess 3509a when the structural element is in the position shown in FIG. 15F.

In some configurations, the screen 3551 may be at an angle of between about 20 degrees and about 60 degrees, optionally between about 25 degrees and about 45 degrees, optionally between about 25 degrees to about 35 degrees relative to a flat horizontal surface when the portable display unit has its support 3581 deployed and the portable display unit display is positioned on a flat surface e.g. a table top. In some examples, the screen may be at about 30 degrees or about 45 degrees in that scenario.

The portable display unit 3000, 3500, 4000 may be provided with handle feature(s) to improve ergonomics and the ability to be handled by a user. The handle feature(s) may comprise one or more recessed regions, shaped regions, or differing/multi-material regions. For example, the impact absorbent material 3511 regions may provide handle features, or may extend along sides of the housing 3501 to assist with gripping the portable display unit. Certain portions of the portable display unit may comprise features which enhance the grip of the screen module by a user.

The handle feature(s) may extend outwardly from the upper face of the display unit housing 3001, 3501, 4001 in the vicinity of the screen 3051, 3551, 4051. For example, the handle feature may comprise a handle above the screen, a handle to one side of the screen, or a handle on either side of the screen. As such, the handle(s) may provide additional drop or impact protection for the screen 3051, 3551, 4051.

The portable display unit 3000, 3500, 4000 may be waterproof and shockproof, and may be capable of withstanding an impact after being dropped from a certain height. In some examples, the portable display unit may be capable of withstanding drops from around 2 m or less which may occur owing to user error. Due to lack of moving parts, the ability to withstand a drop is mostly reliant on strength of materials for the housing.

Consideration of IPX3 to IPX6 protection, may involve internal seals and gaskets to prevent water ingression. In one form, water ingression may not be strictly controlled but a pathway for water to evaporate will be considered. This is owing to the humid working environment.

In a further example, the portable display unit may be hermetically sealed to prevent water and oxygen ingress into the portable display unit.

Exemplary features to provide sealing of the portable display unit 3000, 3500, 4000 are described below for portable display unit 4000. The specification discloses several configurations of a portable display unit 3000, 3500, 4000. Any one or more of the described features and functionality can be used in various combinations in any one more of the portable display units.

In a further example, the portable display unit may be modular e.g. disconnectable to allow various parts to be removed for replacement. The modular display unit allows replacement of the screen or electrical components in the circuit boards used with the display unit.

If the portable display unit 3000, 3500, 4000 will be used in elevated temperature environments, the unit 3000, 3500, 4000 may comprise a heat sink, heat spreader, or heat dissipation element.

The portable display unit 3000, 3500, 4000 may have active or passive cooling to provide heat protection.

The portable display unit 3000, 3500, 4000 may comprise an alarm which is triggered when a threshold temperature (ambient or internal) of the portable display unit 3000, 3500, 4000 is exceeded.

The alarm may be notified by the portable display unit notification light 4150, 4150′, the base unit notification light 240, speaker S, and/or via the screen 3051, 3551, 4051 described below.

The connector 3071, 3571, 4071 of the portable display unit 3000, 3500, 4000 may function as a heat sink when engaged with a complementary connector 2071, 2071′ on the base unit 50 or on a different support component such as a medical pole, incubator, or other component such as those typically used in a medical setting.

The connector 3071, 3571, 4071 and complementary connector 2071, 2071′ may comprise material(s) with high heat transfer capability to transfer heat away from the portable display unit 3000, 3500, 4000 through the connector 3071, 3571, 4071 and complementary connector 2071, 2071′.

The material(s) will typically have a high heat capacity and thermal conductivity to absorb heat energy without shifting towards a very high temperature and transmit it to the environment for efficient cooling. Exemplary materials include aluminium, aluminium alloys, and copper for example.

The display unit housing 3001, 3501, 4001 may comprise fins or similar structures to increase the surface area available for transferring heat to the environment.

The portable display unit 3000, 3500, 4000 may comprise thermal pad(s) to assist with heat sinking or transferring heat from an interior of the portable display unit to an exterior heat sink that is in contact with ambient environment. The thermal pad(s) may provide electrical isolation properties. The thermal pad(s) may comprise silicone or graphite for example.

The display unit housing 3001, 3501, 4001 may comprise cooling vent(s).

The display unit housing 3001, 3501, 4001 may comprise an inner casing and an outer casing. The inner casing may be sealed to prevent oxygen and water ingress, but may comprise sections of conductive material. The outer casing may comprise cooling vent(s) to allow heat dissipation/air flow for convective cooling.

The portable display unit may comprise active cooling. In one example, the active cooling comprises one or more fans.

The fan(s) can be driven by power from the portable display unit battery or via power from the base unit 50.

The fan(s) may be activated after a therapy session has ended, for example as part of a drying cycle. In a drying cycle, the blower of the base unit 50 is powered to a predetermined motor speed for a set time, the heater plate is run at a low power or may be switched off, and the heater wire in the breathing conduit is activated to a predetermined power e.g. full power. The drying cycle lasts for a predetermined period of time.

Similarly, when a drying cycle or a drying mode is activated, a signal may be sent to activate the fan(s) in the portable display unit for a predetermined time, and the fan(s) may be driven at a predetermined motor speed.

The portable display unit 3000, 3500, 4000 may comprise heat-reflective material(s) internally and/or externally of the portable display unit. This may help reflect internal and/or external heat.

A heat-reflective material(s) may form a radiant barrier to reflect heat radiation (radiant heat), preventing or minimising heat transfer from one side of the barrier to another due to a reflective, low emittance surface. The material(s) may comprise reflective tape or foil. The tape or foil may comprise aluminium tape or foil for example. The tape or foil may be coated for resistance to the elements or for abrasion resistance.

As described further herein, internal components of the portable display unit such as the circuit board 4004 may be surrounded by a material which has shock absorption properties. This material may also allow for waterproofing (e.g. by sealing the components) and heat dissipation (by way of its material properties).

When used for sealing, exemplary material may comprise silicone rubber or may comprise any suitable elastomer, such as polyurethane. Alternatively, exemplary material may comprise thermoplastic elastomer(s) and/or thermoplastic vulcanisate(s), particularly if the seal will be overmoulded onto the circuit board 4004.

When used for shock absorbing, exemplary material may comprise shock absorbing polymers, viscoelastic polymers, visco polymers or other polymers. Examples include rubber, neoprene, and silicone.

These materials dissipate impact energy by spreading it over a much larger surface and absorbing it at the same time. This transfers the energy from the impact directly into the material so the internal components are not damaged.

Additionally or alternatively, a potting compound, a foam, or polystyrene could be used.

The display unit housing 3001, 3501, 4001 may comprise one or more features which provide protection against electromagnetic radiation and optionally other types of radiation or interference (such as radio frequency for example). That may comprise shielding on or in the display unit housing 3001, 3501, 4001 to isolate internal components of the portable display unit 3000, 3500, 4000 from the surroundings.

Exemplary materials comprise sheet metal, a metal screen, or metal foam. For example, the material(s) include copper, brass, nickel, silver, steel, tin, aluminium foil, copper foil.

When cable(s) 3061, 3561, 4061 are used, the cable(s) may comprise shielding to isolate internal wire(s) from the external environment.

The portable display unit 3000, 3500, 4000 may be used with, or provided in combined with, an external heat shield. The external heat shield may be configured to be positioned to cover at least part of the portable display unit.

The heat shield may comprise a heat-resistant material. The heat shield may comprise any of the materials outlined above for the heat-reflective material of the portable display unit 3000, 3500, 4000. The material may be transparent.

The heat shield may be mounted to the base unit 50 or to the different support component. In some configurations, the heat shield may be removably or movably mounted to the base unit 50 or to the different support component. For example, when the portable display unit 3000, 3500, 4000 is to be used on a medical pole or medical stand, the portable display unit 3000, 3500, 4000 may be connected to the medical pole or medical stand, and the heat shield may be movably mounted to the medical pole or medical stand to surround or otherwise cover the portable display unit 3000, 3500, 4000.

In some configurations, the heat shield and the portable display unit 3000, 3500, 4000 may be mounted on a pivoting arm. The heat shield and the portable display unit 3000, 3500, 4000 may alternatively be mounted on respective pivoting arms.

The arms or other support components that the heat shield and/or portable display units may be used on may comprise multiple axis pivoting arms, allowing the portable display unit 3000, 3500, 4000 and/or the heat shield to be movably positioned by the clinician or other user.

The heat shield is able to be moved by a clinician or other user to surround or cover the portable display unit and protect it from external heat.

The heat shield may be configured to provide a pocket of air between the heat shield and portable display unit, the pocket of air assisting in insulating heat from the portable display unit 3000, 3500, 4000.

The portable display unit 3000, 3500, 4000 may comprise one or more sensors for ambient conditions; e.g. an ambient light sensor which is able to be used to adjust the brightness of the screen. The portable display unit 3000, 3500, 4000 may comprise a speaker. The speaker may be used for audible alerts.

The portable display unit 3000, 3500, 4000 may comprise or more user input components such as dials, switches or buttons B for control of the breathing assistance apparatus. The dial(s), switch(es) or button(s) B may be separate from the touchscreen. The dial(s), switch(es) or button(s) may provide haptic feedback for the initiation of therapy modes.

The dial, switch or button B can be pressed to activate a function of the base unit 50. Pressing the switch or button B can transmit a signal along the tether either wirelessly or by a wired tether to the base unit 50 to control a component of the base unit. In one example, the dial, switch or button B can be pressed to control the operation of the blower. In this example, the button is pressed and held pressed in order to cause the blower to speed up and increase flow rate delivered or pressure delivered. When the switch or button B is released the blower can be slowed down to reduce flow rate or reduce delivered pressure. Such a button function can be used in manually controlling pressure or flow delivered to the patient. Alternatively, pressing the button can switch off the heater wire or switch off the heater plate.

Because the portable display units 3000, 3500, 4000 are connected to exterior surfaces of the base units 50 of the breathing assistance apparatuses, they can be dimensioned larger than the portion of the base unit that they connect to. For example, the width of the portable display unit 3000, 3500, 4000 is not directly restricted by the width of the screen carrier 211, and can be wider than the screen carrier.

The positioning of the portable display unit 3000, 3500, 4000 when connected to the base unit 50 does not interfere with gases inputs into the base unit 50 or with the breathing conduit 16 to the patient interface.

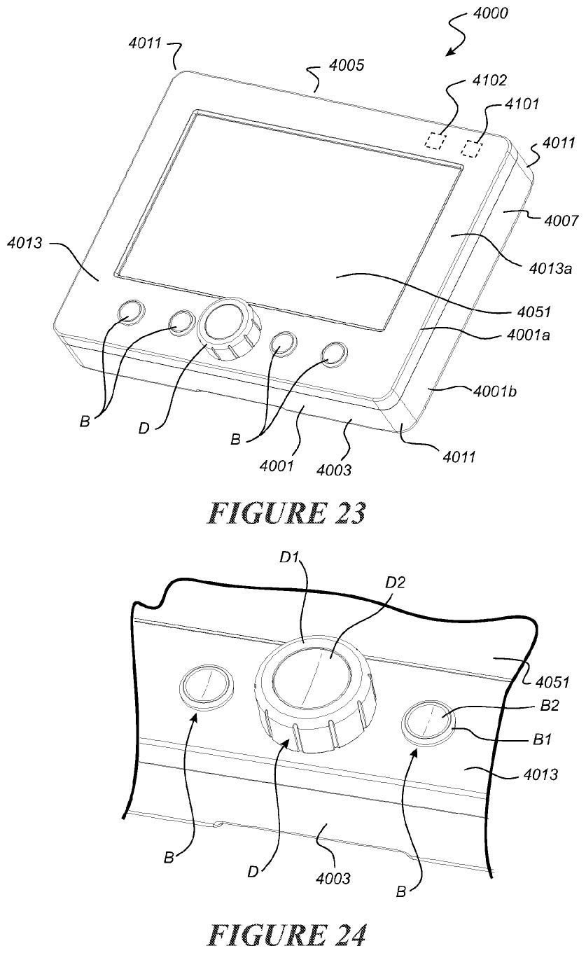

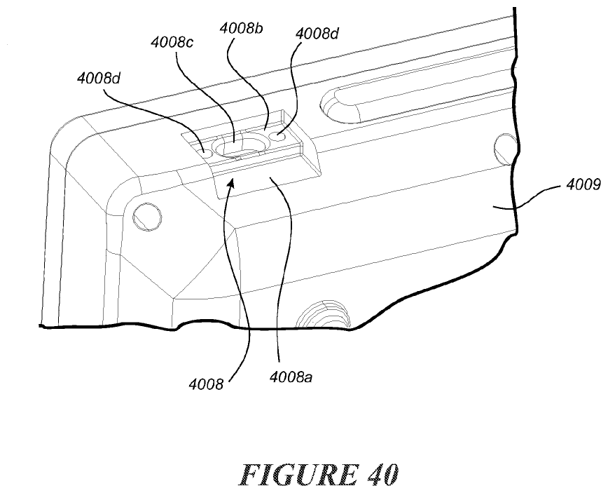

FIGS. 23-30, and 37-41 show an alternative portable display unit 4000 for use with any of the breathing assistance apparatuses described herein. Unless described below, the features, functioning, and options are the same as for portable display unit 3000 or portable display unit 3500, and like reference numerals indicate like parts with the addition of 1000 and 500 respectively.

As outlined above, any one or more described features and functionality can be used in various combinations in any one more of the portable display units 3000, 3500, 4000.

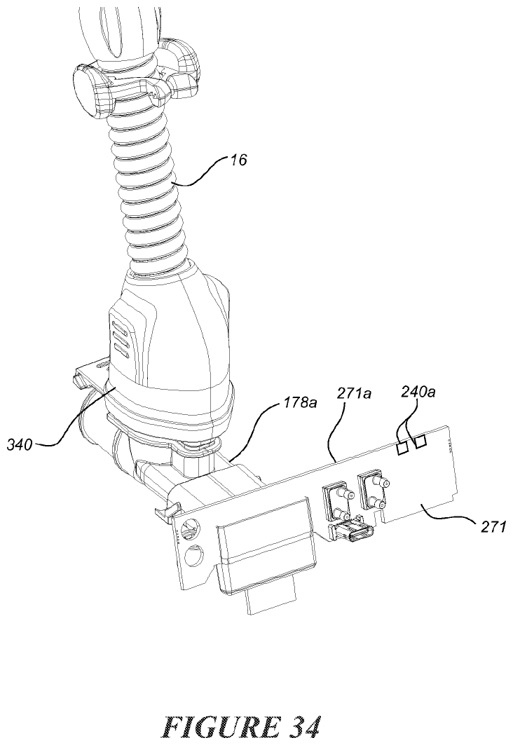

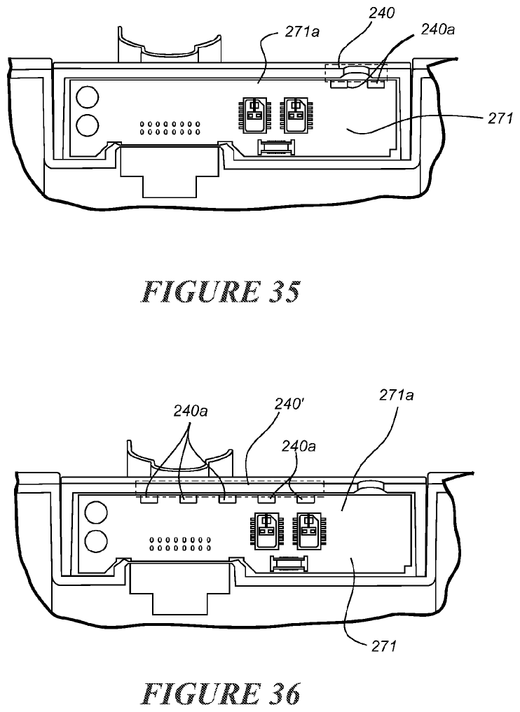

FIGS. 31-37 and 41-45 show additional details of an alternative configuration of the breathing assistance apparatus of FIGS. 5-8. Unless described differently below, the features, functioning, and options are the same as for the breathing assistance apparatus of FIGS. 5-8. Like reference numbers indicate like parts to FIGS. 5-8.

The breathing assistance apparatus of FIGS. 31-37 and 41-45 can be used with any of the portable display units 3000, 3500, 4000 disclosed herein.

In at least some exemplary configurations, a breathing assistance apparatus is provided, the breathing assistance apparatus comprising: a base unit 50; a portable display unit 4000 comprising: a display unit housing 4001; a screen 4051; and at least one connection feature 4071 for removably connecting the portable display unit 4000 to the base unit 50 and to a different support component; and a tethering component for tethering the portable display unit 4000 with the base unit 50.

The different support component may comprise one or more of the support components described with reference to FIGS. 18A-22 for example.

The removable connection of the portable display unit 4000 to the base unit 50 and the different support component may be by way of the same type of connection. Alternatively, the removable connection of the portable display unit 4000 to the base unit 50 and the different support component may be by way of different types of connection.

The tethering component may comprise a cable 4061 for physically tethering the portable display unit 4000 with the base unit 50.

The cable may be for communicating power to the portable display 4000 unit from the base unit 50 and/or for communicating data between the portable display unit 4000 and the base unit 50.

The tethering component TC1 may comprise a component of a wireless communication interface for communicating data between the portable display unit 4000 and the base unit 50.

In at least some exemplary configurations, breathing assistance apparatus comprising: a base unit 50; a portable display unit 4000 comprising: a display unit housing 4001; and a screen 4051; and a tethering component for tethering the portable display unit 4000 with the base unit 50; wherein the base unit 50 comprises at least one complementary connection feature 2071 for cooperating with at least one connection feature 4071 on the portable display unit 4000 for removably connecting the portable display unit 4000 to the base unit 50.

The tethering component may comprise a cable 4061 for physically tethering the portable display unit 4000 with the base unit 50.

The cable may be for communicating power to the portable display 4000 unit from the base unit 50 and/or for communicating data between the portable display unit 4000 and the base unit 50.

The tethering component may comprise a component of a wireless communication interface for communicating data between the portable display unit 4000 and the base unit 50.

The breathing assistance apparatus may comprise a humidifier. In an alternative configuration, the breathing assistance apparatus may not comprise a humidifier.

In the configuration shown, the base unit comprises a recess 108 for receipt of a humidification chamber 300 (not shown in these figures). The features and functionality of the humidifier may be as described herein.

The humidifier is configured to be placed below patient head height.

In some configurations, the humidifier is configured to be placed below patient level or below patient bed level.

The base unit/humidifier will be the lowest point when the breathing assistance apparatus is set up for use.

The breathing assistance apparatus may further comprise a breathing circuit in fluid communication with the humidifier, the breathing circuit comprising a patient interface to deliver gases to a patient.

The breathing circuit and humidifier are configured such that when the humidifier is placed below patient head height, any condensate which accumulates in the breathing circuit is encouraged to return to the humidifier rather than collecting at the patient interface. The breathing circuit may be any of the types described herein.

The portable display unit 4000 is configured to be placed above the base unit 50 and/or the humidifier.

The portable display unit 4000 and the humidifier are configured such that when the display unit is above humidifier, the portable display unit 4000 enables user interaction and control of the breathing assistance apparatus from an elevated position above the humidifier, optionally at or near eye level of a user.

The elevated position may be separate from the base unit 50 and vertically higher than the base unit 50 and the humidifier.

The at least one connection feature 4071 may be engageable with at least one complementary connection feature 2071 of the base unit 50.

The at least one connection feature 4071 of the portable display unit 4000 may comprises a first connection component 4071′.

The complementary connection feature 2071 of the base unit may comprise a second connection component on the base unit 50 and/or on the different support component.

The first connection component and the second connection component may be components of a sliding connector.

The sliding connector may comprise a dovetail connector.

The first component 4071′ and second component 2071 may be engageable with each other in a sliding action of one component relative to the other. For example, the first component 4071′ may be engageable with the second component 2071 in a sliding action of one component relative to the other.

In one exemplary configuration, the sliding action of the first component 4071′ may be forwardly relative to the second component 2071.

Once engaged, the first component 4071′ and second component 2071 may not be disengageable by attempted movement of the first component 4071′ relative to the second component 2071 in a direction transverse to the first direction. The first component 4071′ may only be disengageable from the second component 2071 by moving the first component 4071′ relative to the second component 2071 in a second direction that is opposite to the first direction.

In one exemplary configuration, the second direction is rearward.

One of the first component 4071′ and the second component 2071 may comprise a male component, and the other of the first component 4071′ and the second component 2071 may comprise a complementary female component to receive the male component. For example, the male component may be provided on the portable display unit 4000 and the female component may be provided on the base unit 50 or on the different support component. Alternatively, the female component may be provided on the portable display unit 4000 and the male component may be provided on the base unit 50′ or on the different support component.

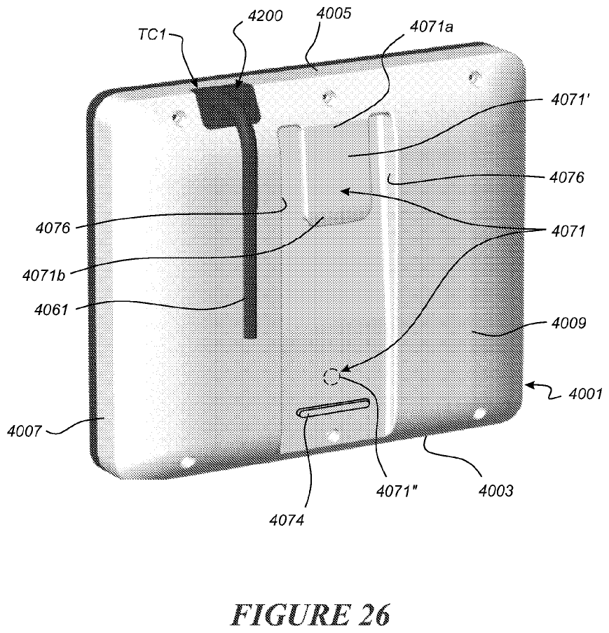

FIG. 26 shows an exemplary configuration of the first component 4071′. FIGS. 31-33 show an exemplary configuration of the second component 2071.

The first component 4071′ will typically be positioned on or in the base 4009 of the display unit housing.

The first component 4071′ could be provided in any suitable position on the display unit housing 4001. For example, towards the rear wall 4005 of the display unit, towards the front wall 4003 of the display unit, and/or towards one or other of the side walls 4007 of the display unit housing 4001.

For example, to interface with the second component 2071 shown in FIGS. 31-33, the first component 4071′ may be positioned towards the front wall 4003 of the display unit compared with what is shown in FIG. 26.

The first component 4071′ could project from the display unit housing 4001 to enable easier connection, or could be recessed in the display unit housing 4001 to provide a more slimline configuration.

The second component 2071 could project from the base unit 50 or could be recessed into the base unit 50. The second component 2071 could be provided in any suitable location on the base unit.

The same options are available for any of the other described connector component(s) and complementary connector component(s).

In the configuration shown, the first component 4071′ comprises a tongue and the second component 2071 comprises a receiver or cradle for the tongue. The receiver may act as a cradle to hold part of the tongue when the first component 4071′ is connected to the second component 2071.

The first component 4071′ (i.e. the tongue) and the second component 2071 (i.e. the receiver or cradle) may comprise components of a dovetail connector.

The tongue has a proximal portion 4071a connected to the display unit housing 4001 and a distal portion 4071b. The tongue may be connected to the display unit housing 4001 along the length of the tongue or alternatively may only have the proximal end 4071a connected to the display unit housing 4001. The tongue may be integrally formed with the display unit housing 4001 or may be fastened to the display unit housing 4001.

The receiver comprises a forward wall 2075, two side walls 2077, and two inwardly directed flanges 2079. The flanges 2079 are spaced apart to provide a relatively narrow spacing 2081 between the flanges. An end of the housing opposite the forward wall 2075 defines a relatively wide opening 2083′ that is wider than the spacing 2081.

The portable display unit 4000 connects to, and separates from, the housing of the base unit 50 by a sliding action of the portable display unit relative to the base unit. In the configuration shown, the distal portion 4071b of the first component 4071′ is inserted into the connection housing under the flanges 2079 and slid forwardly, so that the distal portion 4071b is substantially received within the flanges with the proximal portion 4071a positioned between the flanges. Because the connection feature on the base unit 50 is angled, gravity will assist with retaining the portable display unit 4000 in connection with the base unit 50. To separate the portable display unit 4000 from the base unit, the portable display unit is slid rearwardly relative to the screen carrier 211 until the first component 4071′ clears the flanges 2079.

The connection may be such that a user can lift the base unit 50 by lifting the portable display unit 4000 while it is connected to the base unit 50.

In an alternative configuration, the first component 4071′ could have the configuration shown and described for the connection feature 3071. The functionality of this embodiment is substantially the same as described.

The breathing assistance apparatus may comprise a display mounting component 2072 that comprises the complementary connection feature 2071.

The display mounting component 2072 is connectable to the base unit 50.

The display mounting component 2072 may be connectable to the screen carrier 211 of the base unit 50, or may be connectable to the main housing 100 of the base unit 50. The display mounting component 2072 may be removably connected to the main housing 100 or the screen carrier 211 of the base unit 50.

The display mounting component 2072 may connect to the base unit 50 by way of fasteners, integrated clip features, or the like.

The display mounting component 2072 may comprise a cable receptacle 2072a for receipt of the cable 4061 which enters or is otherwise secured to the base unit 50 by way of the display mounting component 2072. Removal of the display mounting component 2072 may allow the cable 4061 to be disconnected from the base unit 50 and replaced.

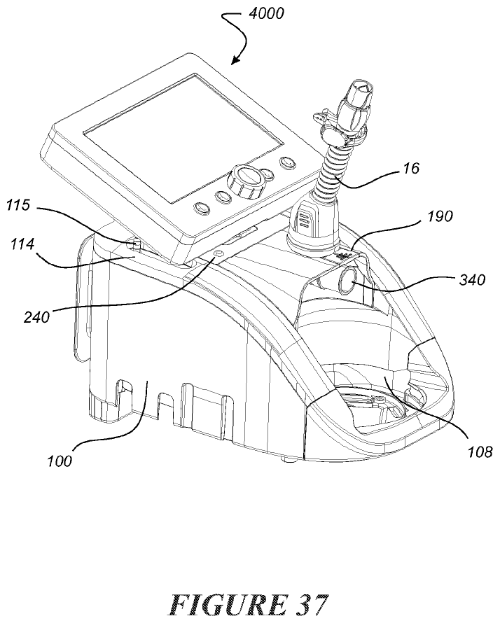

As shown in FIGS. 31 and 37, the display unit mounting component 2072 enables the upper surface 211a of the screen carrier 211 to be relatively horizontal and low profile, with the display mounting component 2072 providing height and optionally an increased angle for the portable display unit 4000 when connected to the screen mount component 2072.

When a relatively steep display mounting component 2072 is used, the first component 4071 may be provided near the front wall 4003 of the portable display unit 4000 to provide clearance. When a relatively flat display mounting component 2072 is used, the first component 4071 may be provided in the position shown in FIG. 26 or may be provided elsewhere on the portable display unit 4000.

The display mounting component 2072 and the base unit 50 (e.g. the screen carrier 211 or the main housing 100) may comprise complementary tongue and groove engagement features that engage with each other to inhibit water ingress into the main housing 100.

In an alternative configuration, the complementary connection feature 2071 may be provided as part of the screen carrier 211 or main housing 100. That is, the complementary connection feature 2071 may be integrally formed as part of the screen carrier 211 or main unit housing 100. FIG. 44 shows an exemplary configuration of that arrangement.

In this configuration, the cable 4061 may be attached to the main housing 100 or screen carrier 211.

In an alternative configuration of the portable display unit 4000, the first component 4071 may have the configuration described for the connection feature 3071 or the connection feature 3571.

The portable display unit 4000 may comprise a retention feature 4074 that is configured to positively engage with a complementary retention feature 2074 on the base unit 50 upon connection of the portable display unit 4000 with the base unit 50.

Therefore, the portable display unit 4000 and the base unit 50 may comprise complementary retention features 4074, 2074 (FIGS. 26 and 44) that positively engage with each other upon connection of the portable display unit 4000 with the base unit 50. The retention feature 4074 of the portable display unit 4000 is configured to engage with the complementary retention feature 2074 of the base unit 50 upon connection of the portable display unit 4000 with the base unit 50.

The retention features 4074, 2074 positively engage with each other.

The retention features 4074, 2074 may require disengagement to enable disconnection of the portable display unit 4000 from the base unit 50. The retention features 4074, 2074 may be selectively releasable from engagement with each other to enable disconnection of the portable display unit 4000 from the base unit 50.

In an exemplary configuration, the retention feature 4074 on the portable display unit 4000 comprises a projection or recess that is engageable with a complementary projection or recess 2074 on the base unit 50.

The retention features 2074, 4074 may comprise a clip or buckle-type structure that is engageable with a corresponding clip or buckle portion on the base unit. The retention features 2074, 4074 may be such that they require the user to physically press or engage one of the features from the other in order to releasably engage or disengage the features from one another.

The retention features could have any suitable shape, such as elongate, round, or polygonal for example.

The retention features 4074, 2074 may be provided on the connection features 4071, 2071 or may be provided adjacent the connection features 4071, 2071 or elsewhere on the portable display unit 4000 and base unit 50.

For example, one of the retention features 4074 may be provided on the tongue of the first connection feature 4071 and the other retention feature 2074 may be provided on the receiver of the second connection feature 2071.

In an alternative configuration, the first retention feature 4074 may be provided on the portable display unit housing 4001, and the second retention feature 2074 may be provided on the base unit 50 such as on the screen carrier 211 or the main housing 100 for example.

The retention feature 4074, 2074 on the portable display unit 4000 or on the base unit 50 may be provided on a selectively movable portion of the portable display unit 4000 or base unit 50, Selective movement of the selectively movable portion may disengage the retention features 4074, 2074 from each other to enable the connection feature 4071 and complementary connection feature 2071 to be disengaged from each other.

In the configuration shown in FIG. 44, the retention feature 2074 may be provided on the screen carrier 211 at or towards a forward edge thereof. Applying a downward force in direction DF at a location at or adjacent to the retention feature 2074 will disengage the retention features 4074, 2074 from each other. That will enable the connection feature 4071 and complementary connection feature 2071 to be disengaged from each other by sliding the portable display unit 4000 rearward relative to the base unit 50.

The location at which the downward force DF is to be applied will be exposed for access when the portable display unit 4000 is connected to the housing 50.

When the complementary connection feature 2071 is used on a different support component, the retention feature 2074 may be provided on that different support component.

The retention features 4074, 2074 may provide enhanced frictional engagement between the portable display unit 4000 and the base unit 50, and/or may provide haptic and/or audible feedback of full connection of the portable display unit 4000 and the base unit 50.

The same connection feature 4071 and complementary connection feature 2071 may be used for connecting the portable display unit 4000 with the base unit 50 and with the different support component. Alternatively, different connection features may be used.

As outlined above, an additional or alternative connection feature 4071 may comprise magnet(s) and may predominantly use magnetic force for the connection or may use magnetic force to bias the connection.

For example, one of the portable display unit 4000 and the base unit 50/different support component may comprise one or more magnets, and the other of the portable display unit 4000 and the base unit 50/different support component may comprise one or more magnets or magnetisable material. The magnet(s) on the portable display unit 4000 or base unit 50/different support component may cooperate with the magnet(s) or magnetisable material on the other of the portable display unit 4000 and base unit 50/different support component.

In an exemplary configuration, the at least one connection feature 4071 may further comprise a magnet 4071″.

The magnet 4071″ is configured to cooperate with a magnet or a magnetisable material 2071″ on the base unit 50 (FIG. 31) and/or on the different support component.

In an alternative configuration, the base unit 50 and/or different support component may comprise a magnet and the portable display unit 4000 may comprise a magnet or magnetisable material. This may be provided in addition to the connector 4071 and complementary connector component 2071. That may strengthen the connection provided by the connector 4071 and the complementary connector component 2071.

In some configurations, the at least one connection feature 4071 comprises a first connection feature 4071″ for removably connecting the portable display unit to the base unit 50 and comprises a second connection feature 4071′ for removably connecting the portable display unit 4000 to the different support component, wherein the first connection feature is different to the second connection feature.

In some configurations, the first connection feature 4071″ is configured to provide a relatively weak connection of the portable display unit 4000 to the base unit 50 and the second connection feature 4071′ is configured to provide a relatively strong connection of the portable display unit 4000 to the different support component.

With this configuration, the portable display unit 4000 can be easily disconnected from the base unit 50 so that lifting the portable display unit 4000 will not cause the base unit 50 to inadvertently be moved or lifted off a support surface, which may be important when therapy is being provided, as movement of the base unit may affect the therapy provided. It may also enable the portable display unit 4000 to be accessed easily and used during emergency situations, for example the relatively weak connection provided by the first connection feature 4071″ allows for the portable display unit 4000 to be quickly removed from the base unit 50 without the disengagement and/or removal of the portable display unit 4000 from the base unit 50. Additionally, the portable display unit 4000 is unlikely to inadvertently disconnect from the different support component by way of the relatively strong connection provided by the second connection feature 4071′, which reduces the likelihood of the portable display unit 4000 being disconnected or dropped from the different support component. This is particularly advantageous if the different support component is a portable component such as a medical pole for example.

For example, the first connection feature 4071″ may comprise part of the magnetic connection arrangement discussed above, and the second connection feature 4071′ may comprise a first component of a sliding connector as discussed above.

Accordingly, in an exemplary configuration the first connection feature 4071″ comprises a magnet configured to cooperate with a magnet or a magnetisable material on the base unit 50. Alternatively, the first connection feature 4071″ may comprise a magnetisable material configured to cooperate with a magnet on the base unit 50.

The second connector feature 4071′ in this configuration may comprise a first component of the sliding connector that is engageable with a second component of the sliding connector on the different support component. The different support component may comprise the second component of the sliding connector described above, such as the receiver for example.

The first connection feature 4071″ may provide an additional secondary connection for the different support component. That is, the magnetic connection may not be the primary connector on the different support component, but may add to the strength of the connection provided by the second connection feature 4071′.

Rather than being provided on the base 4009 of the portable display unit 4000 and the upper surface 211a of the screen carrier 211, the connection features 4071, 2071 could be provided elsewhere on the portable display unit 4000 and/or base unit 50, with the connection and removal directions varying accordingly.

The breathing assistance apparatus may comprise at least one alignment feature to assist with aligning the portable display unit 4000 with the base unit 50 during connection of the portable display unit 4000 to the base unit 50.

The at least one alignment feature may be provided on the base unit 50 and/or on the portable display unit 4000.

The at least one alignment feature may comprise an alignment surface on the base unit 50 that is configured to interact with a complementary alignment surface on the portable display unit 4000.

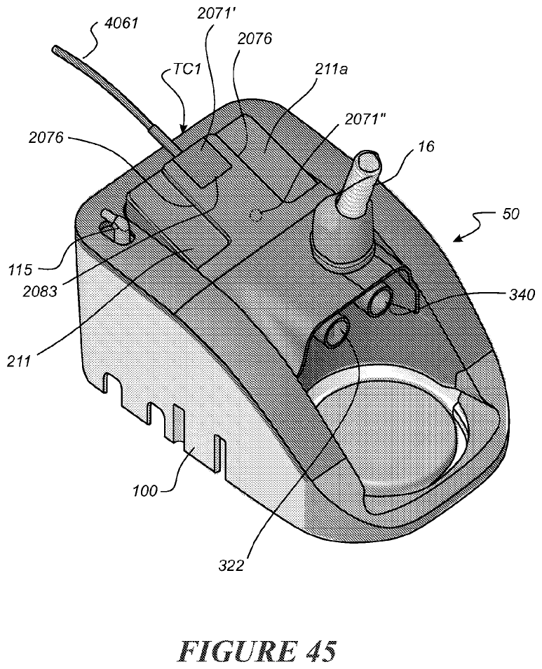

For example, in the configuration of FIGS. 26 and 45, the base unit 50, and optionally the display mounting component 2072 of the base unit 50, comprises at least one alignment surface 2076 that is configured to interact with at least one complementary alignment surface 4076 on the portable display unit 4000.

The base unit 50 and the portable display unit 4000 may each comprise one, two, three, or more complementary alignment surfaces.

When a sliding connector is used, the alignment surfaces 2076, 4076 are oriented to be parallel to a sliding connection and disconnection direction of the portable display unit with the base unit 50.

In the configuration shown, the alignment surfaces 2076 are provided by side walls of the receiver of the base unit, and the alignment surfaces 4076 are provided by walls of a channel or groove in the base 4009 of the display unit housing 1004. The alignment surfaces could alternatively be provided by other walls or members on the base unit 50 and the portable display unit 4000. For example, the alignment surfaces could be provided by protrusion(s) on the base unit 50 or portable display unit to interact with complementary protrusion(s) or recess(es) on the other of the base unit 50 and the portable display unit 4000.

Channels may be provided between the complementary alignment surfaces 4076 and the tongue of the connector 4071 to provide a space for receipt of the walls of the receiver of the base unit.

FIG. 45 shows an alternative configuration of the base unit 50. In this configuration, the receiver 2071′ is configured to receive the connector 4071. However, the receiver 2071′ does not comprise the flanges 2079 of FIG. 44. Instead, the receiver 2071′ comprises an enlarged region 2083 along its length.

The tongue of the connector 4071 is configured to be received in the receiver 2071′ of FIG. 45 when the portable display unit 4000 is connected to the base unit 50 via the magnetic arrangement comprising the magnet(s) and/or magnetisable material.

In this configuration, the receiver 2071′ act as an alignment feature/locator for the tongue of the connector 4071, but not as the primary connection feature between the portable display unit 4000 and the base unit 50.

The tongue of the connector 4071 may comprise the magnet and the receiver 2071′ may comprise the magnet or magentisable material, or vice versa.

In addition this alignment feature/locator, the base unit 50 may have one or more alignment features 2076 as discussed above.

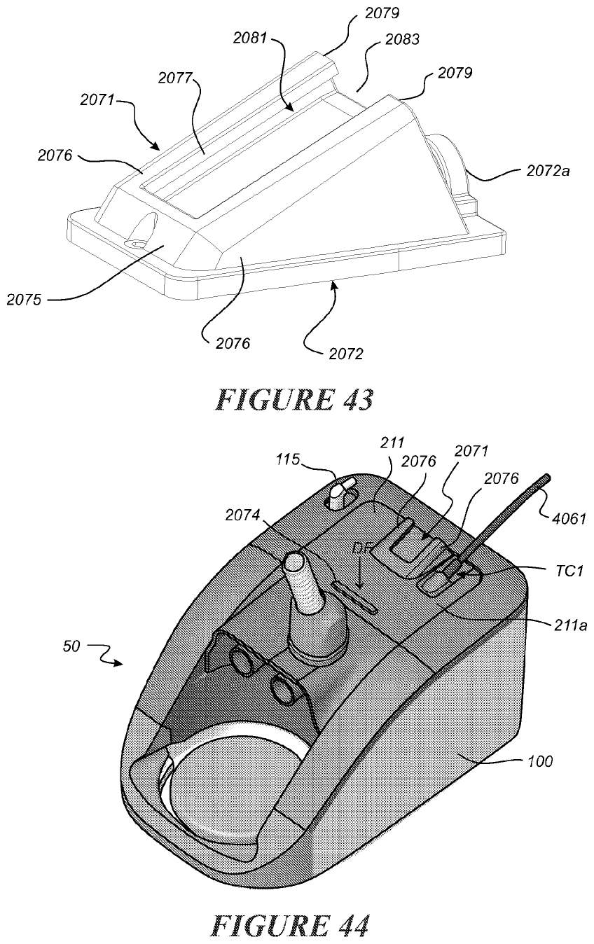

The receiver 2071 of FIG. 45 can be provided on the main housing 100, screen carrier 211, or on a display unit mounting 2072 of the type described above with reference to FIG. 43.

In the configurations shown for the base unit 50, the base unit does not have a handle portion 506 or lever 500. In alternative configurations, a handle portion or lever may be provided.

In the configurations shown for the portable display unit 4000 and base unit 50, space is maximised for the portable display unit 4000. As shown in FIG. 37 for example, the portable display unit 4000 can be at least as wide as the main housing 100 of the base unit 50 and may be large enough to project beyond a rear edge of the main housing 100 of the base unit. The positioning of the portable display unit 4000 when connected to the base unit 50 does not interfere with gases inputs into the base unit 50′ or with the patient conduit 16 to the patient interface.

The base unit 50 may comprise a filter inlet 115 for delivery of gases into a filter of the base unit. The filter inlet 115′ may be provided at or adjacent a rear corner of the main housing 100 of the base unit 50. The filter inlet 115 may be located under a portion of the portable display unit 4000 when the portable display unit 4000 is connected to the base unit 50.

In some exemplary configurations, the portable display unit 3000, 3500, 4000 may be provided with a notification light. In some configurations, a portable display unit 4000 for a breathing assistance apparatus is provided, the portable display unit comprising: a display unit housing 4001; a screen 4051; at least one connection feature 4071 for removably connecting the portable display unit 4000 to a base unit 50 of the breathing assistance apparatus; and a notification light 4150, 4150′.

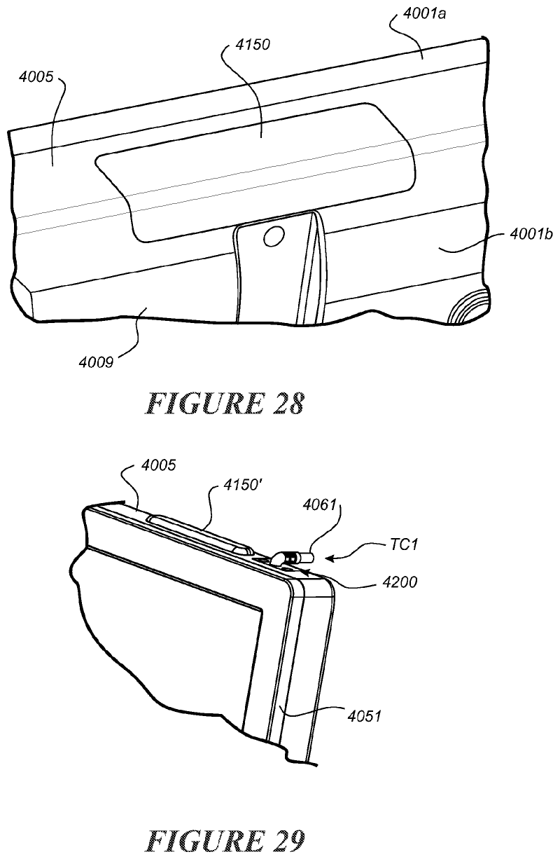

An exemplary configuration is shown in FIGS. 28-30.

The display unit housing 4001 comprises a front wall 4003, a rear wall 4005, opposed side walls 4007, and a base wall 4009.

In the configuration shown, the notification light 4150, 4150′ is provided in or on at least one external surface of the display unit housing 4001. The notification light 4150, 4150′ may project outwardly from the external surface(s) for better visibility.

The notification light could be on or in any wall/surface or any combination of walls/surfaces.

As one example, in the configuration shown in FIG. 28, the notification light 4150 is provided in a rear wall 4005 and base wall 4009 of the display unit housing 4001. As another example, in the configuration shown in FIG. 29, the notification light 4150′ is provided in the rear wall 4005 of the display unit housing 4001.

The notification light 4150, 4150′ may be visible from at least a portion of the portable display unit comprising the screen 4051. In some configurations, the display unit housing 4001 comprises a transparent portion in an upper surface 4013 of the display unit housing, and the notification light is visible through the transparent portion. The transparent portion could be in any surface or combination of surfaces.

In some configurations, the notification light 4150, 4150′ projects from a rear of the display unit housing 4001, such that the notification light 4150, 4150′ is visible from at least a rear, an upper surface, a left side, a right side, and a base of the portable display unit. The notification light could project from any surface or combination of surfaces.

In some configurations, the notification light is visible from at least a rear, an upper surface, a left side, a right side, and a base of the portable display unit.

The notification light 4150, 4150′ may be visible from substantially a 360 degree viewing angle around the portable display unit 4000.

The notification light 4150, 4150′ may be provided within the display unit housing 4001. The display unit housing 4001 may comprise a transparent portion, such that the notification light is visible through the transparent portion.

In some configurations, the display unit housing 4001 comprises at least one transparent portion, and wherein the notification light 4150 is visible through the at least one transparent portion so as to be visible from 360 degrees around the display unit housing 4001. The transparent portion(s) may protrude from the display unit housing 4001 to provide the 360 degree visibility.

The notification light 4150, 4150′ may be configured to provide a visual alert in response to an alarm condition.

The alarm condition may comprise an alarm condition of the portable display unit 4000 and/or an alarm condition of the breathing assistance apparatus.

Exemplary alarm conditions of the breathing assistance apparatus include one or more of: can't reach target oxygen, oxygen detected when therapy off, SpO2 below target, high leak, chamber leak, empty chamber, no mains power, patient end temperature sensor readings are above a threshold temperature, heater plate temperature sensor readings are above a threshold temperature.

Exemplary alarm conditions of the portable display unit include one or more of: disconnection of a cable 4061 between the portable display unit 50 and a base unit of the breathing assistance apparatus, physical or wireless communication disconnection from the breathing assistance apparatus, poor communications latency with the breathing assistance apparatus.

The notification light 4150, 4150′ may comprise one or more light elements. For example, the notification light 4150, 4150′ may comprise one or more LEDs. In an alternative configuration, the notification light 4150, 4150′ may comprise a backlight of the screen 4051 or user input components such as the button(s) B. In that configuration, the display unit housing may comprise the transparent portion such that the backlight becomes visible during a visual notification.

The notification light 4150, 4150′ may comprise a plurality of lights of different colours. For example, the notification light 4150, 4150′ may comprise a plurality of LEDs of different colours, or may comprise multicolour/RGB LEDs.

LEDs may be provided in a row such that the LEDs are able to be lit in a sequence or pattern which is used by the user or clinician to identify an alarm condition. The pattern or sequence may light the multiple LEDs in a sequence of illuminated/unilluminated to form a recognisable and/or repeated pattern, the pattern or sequence may additionally or alternatively define the lighting of the LEDs in different colours which define a pattern.

The notification light 4150, 4150′ may be configured as an elongate light bar that extends along a portion of at least one of the walls of the display unit housing 4001. In some embodiments the elongate light bar may project outwardly from the display unit housing 4001.

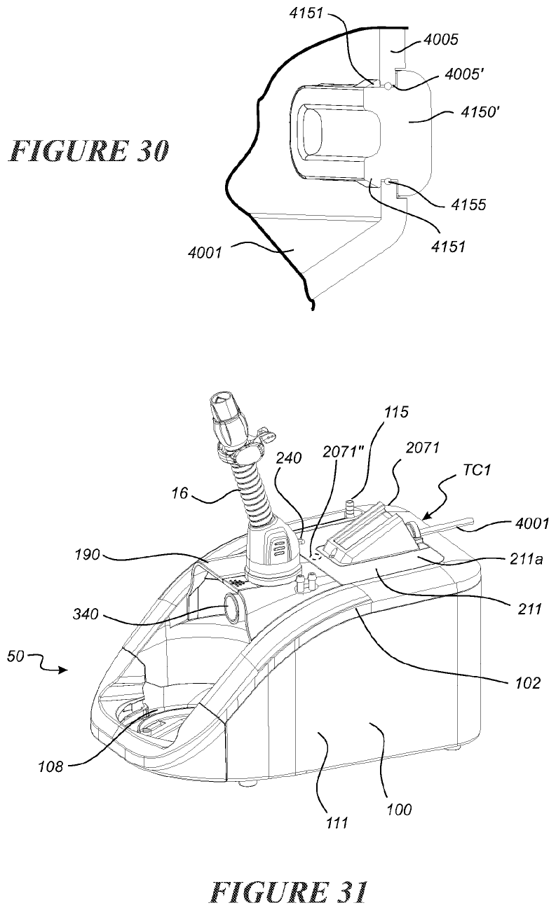

The notification light 4150, 4150′ may be connected to the display unit housing via a clip configuration, or via fasteners or adhesive for example. FIG. 30 shows an exemplary clip configuration. In that configuration, two opposed protrusions 4151 engage against wall edges 4005′ of the wall 4005 of the display unit housing 4001. The protrusions 4151 may be tapered to enable relatively easy engagement of the notification light 4150′ with the wall, but to inhibit disengagement of the notification light 4150′ from the wall.

A seal or gasket 4155 such as an o-ring for example may be provided between the notification light 4150, 4150′ and the display unit housing 4001 to assist with hermetically sealing the display unit housing against water and/or oxygen ingress.

The seal or gasket may comprise silicone or any other suitable material.

The portable display unit may further comprise a notification speaker S as shown schematically in FIG. 27.

In an alternative configuration, the portable display unit may comprise a speaker but no notification light.

The notification speaker S is configured to provide an audio alert in response to an alarm condition.

The alarm condition may comprise an alarm condition of the portable display unit 4000 and/or an alarm condition of the breathing assistance apparatus.

Exemplary alarm conditions of the breathing assistance apparatus include one or more of: can't reach target oxygen, oxygen detected when therapy off, SpO2 below target, high leak, chamber leak, empty chamber, no mains power, patient end temperature sensor readings are above a threshold temperature, heater plate temperature sensor readings are above a threshold temperature.

Exemplary alarm conditions of the portable display unit include one or more of: disconnection of a cable 4061 between the portable display unit and a base unit 50 of the breathing assistance apparatus, physical or wireless communication disconnection from the breathing assistance apparatus, poor communications latency with the breathing assistance apparatus, or that the portable display unit is no longer in close proximity with the base unit or has moved out of a predefined acceptable range from the base unit (this could be indicated by the latency between the display and the base unit exceeding a threshold for example).

In some configurations, the notification light 4150, 4150′ and the notification speaker S are configured to operate in combination to provide alerts. That is, when the notification light 4150, 4150′ issues a visual alert the notification speaker S may issue an audible alert.

The portable display unit with a notification light 4150, 4150′ and optionally with a notification speaker S may be used with, or provided in combination with, a breathing assistance apparatus having a base unit 50.

In some configurations, the combination of a breathing assistance apparatus comprising a base unit 50 with a housing 100 having a gas port 21 and the above portable display unit 4000 is provided. An exemplary configuration is shown in FIG. 31-37.

The base unit 50 may comprise a base unit notification light 240.

In the configuration shown, the base unit notification light 240 is provided in or on at least one external surface of the base unit 50.

The base unit notification light 240 may be provided within the housing 100 or screen carrier 211 or shroud 190 of the base unit 50. The housing 100 or screen carrier 211 or shroud 190 of the base unit 50 may comprise a transparent portion, and the notification light 240 may be visible through the transparent portion.

As one example, in the configuration shown, the base unit notification light 240 is provided on or in an upper wall of the base unit. For example, the base unit notification light 240 may be provided in an upper wall of the screen carrier 211. Alternatively, the base unit notification light 240 may be provided in an upper wall of the main housing 100. As another alternative, the base unit notification light 240 may be provided in a different wall of the screen carrier 211 or the main housing 100.

The base unit notification light 240 may project outwardly from the base unit.

The base unit notification light 240 may be visible from at least a top of the base unit.

As shown in FIG. 37, the base unit notification light 240 is advantageously visible from an upper forward region of the base unit 50 when the portable display unit 4000 is connected to the base unit 50.

The base unit notification light 240 may be configured to provide a visual alert in response to an alarm condition.

The alarm condition may comprise an alarm condition of the portable display unit 4000 and/or an alarm condition of the breathing assistance apparatus.

Exemplary alarm conditions include one or more of: disconnection of a cable 4061 between the portable display unit 4000 and a base unit 50 of the breathing assistance apparatus, physical or wireless communication disconnection from the breathing assistance apparatus, poor communications latency with the breathing assistance apparatus.

In some configurations, the portable display unit notification light 4150, 4150′ and the base unit notification light 240 are configured to operate in combination to provide alerts. That is, when the portable display unit notification light 4150, 4150′ issues a visual alert the base unit notification light 240 may issue a corresponding or the same visual alert. They may issue those alerts in combination with the audible alert from the notification speaker S.

The base unit notification light 240 may comprise one or more light elements 240a. For example, the base unit notification light 240 may comprise one or more LEDs.

The base unit notification light 240 may be configured as an elongate light bar that extends along or across a portion of the base unit 50; for example, across a forward portion of the screen carrier 211 of the base unit. An exemplary configuration light bar 240′ is shown in FIG. 36.

The base unit notification light 240 may be configured to indicate different colours in response to different notification conditions. The notification conditions may relate to the portable display unit 4000.

For example, the base unit notification light 240 may indicate light of a first colour (e.g. green) to indicate that the portable display unit 4000 is properly connected to the base unit 50, and may indicate light of a second colour (e.g. red) to indicate that the portable display unit 4000 is not properly connected to the base unit 50. As another example, the base unit notification light 240 may indicate lights of different colours for when there is power connection between the base unit and the portable display unit but not data connection, when there is data connection between the baes unit and the portable display unit but no power connection.

When the base unit notification light 240 comprises a plurality of light elements 240a, the light elements may have different colours from each other to indicate the different notification colours.

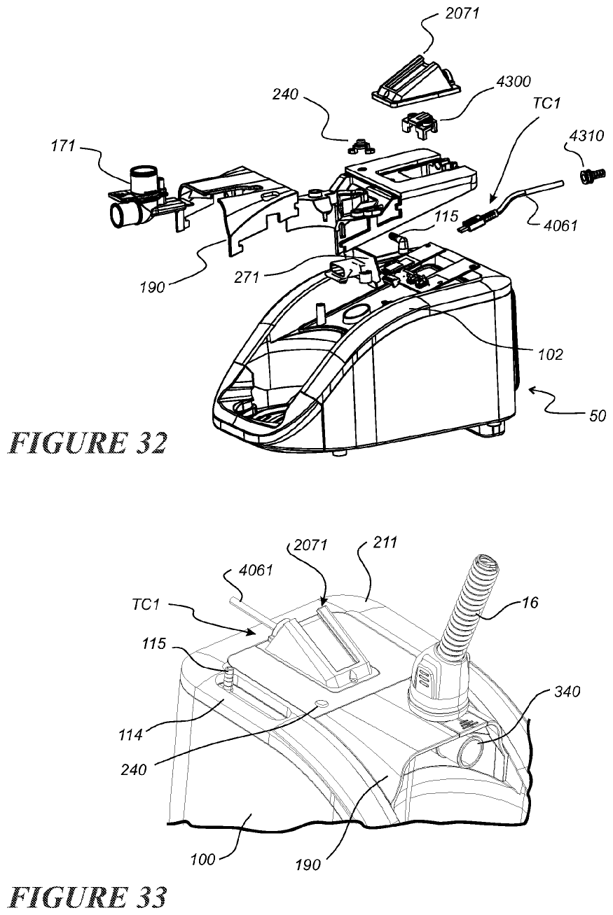

As shown in FIGS. 32 and 34-36 for example, the base unit 50 may comprise an interconnect circuit board 271 for providing data communication between the portable display unit and a main circuit board 272 (FIG. 4D) of the base unit 50. The interconnect circuit board may be a printed circuit board. The base unit notification light 240 may be coupled to the interconnect circuit board 271.

The light elements 240a may be coupled to, or provided on, the interconnect circuit board 271.

In one exemplary configuration, the light elements 240a are coupled or mounted at or adjacent a top edge 271a of the interconnect circuit board 271.

The interconnect circuit board 271 may allow for electrical and/or electronic connection with the portable display unit 4000 and the patient conduit 16. The interconnect circuit board 271 may include other electrical connectors for different conduits or connectors.

The interconnect circuit board 271 is in electrical communication with the main circuit board 272 and controls the connections to the portable display unit 4000, patient conduit 16, and other conduits or connectors as applicable.

The interconnect circuit board 271 has a cable connector 271b for receipt of the cable 4061 of the portable display unit 4000. The cable 4601 may comprise a connector 4601c to connect to the cable connector 271b. The cable connector 271b may be a power connector, a data connector, or a power and data connector. In one exemplary configuration, the cable connector 271b is a USBC connector.

In some configurations, the portable display unit 4000 may comprise a button B, and a dial D. In some configurations, a portable display unit 4000 for a breathing assistance apparatus is provided, the portable display unit 4000 comprising: a display unit housing 4001; a screen 4051; a cable physically tethering the portable display unit to a base unit of the breathing assistance apparatus; a button B; and a dial D.

In some configurations, the cable 4061 is for communicating power to the portable display unit 4000 from the base unit 50 and/or for communicating data between the portable display unit 4000 and the base unit 50.

The button B may provide a first user input component and the dial D may provide a second user input component.

The tethering component may provide a physical tether or a wireless tether of any of the types described herein.

An exemplary configuration of the portable display unit 4000 is shown in FIGS. 23-25.

The portable display unit 4000 may comprise one button B or may comprise a plurality of buttons B. In the configuration shown, the portable display unit comprises four buttons; however, the portable display unit 4000 may comprise one, two, three, four, or more buttons.

The button(s) B may be provided on any suitable surface of the portable display unit to enable user interaction with the button(s). In the configuration shown, the button(s) B are provided on the upper surface 4013 of the display unit housing 4001, adjacent to the screen 4051.

The screen 4051 may be a touch screen as outlined above, to enable the user to interact with the screen to control one or more functions of the breathing assistance apparatus.

The button(s) may be electrical push button(s). Alternatively, the button(s) B may be membrane button(s).

Membrane button(s) may help with preventing water and oxygen ingress into the interior portion of the display unit housing.

The button(s) may be backlit.

The membrane button(s) may comprise a raised edge B1 and a centre portion B2 for pressing for interaction by a user. The centre portion B2 may be recessed from the raised edge B1 to minimise the likelihood of inadvertent actuation of the button(s).

The button(s) B may provide haptic feedback. The haptic feedback may be indicative of initiation of therapy modes of the breathing assistance apparatus.

The dial D comprises a rotatable component D1 to provide a user input.

In the configuration shown, the rotatable component D1 comprises a rotatable knob.

The rotatable component D1 is configured to be actuated by a user rotating the rotatable component. The rotatable component D1 is only movable with a rotating action, and is not movable in an axial direction of the dial.

The dial D further comprise a push button D2 to provide a different user input from the rotatable component.

The push button D2 is configured to be actuated by a user pushing the push button D2 in an axial direction towards the display unit housing 4001.

The rotatable component D1 surrounds a periphery of the push button D2. A pushing surface D2′ of the push button D2 is recessed within the rotatable component D1.

As a result, the rotatable component D1 provides protection for the push button D2 by housing the push button D2 inside it, thereby providing impact protection for the push button D2 and reducing the likelihood of inadvertent actuation or damage of the push button D2 if a force is applied in an axial direction of the dial D. Forces applied generally in an axial direction of the dial D, unless specifically applied to only the push button D2, will instead be transmitted through the rotatable component D1 and then through the display unit housing 4001, rather than through the push button D2 to the circuit board 4004 in FIG. 25.

Additionally, because the pushing surface D2′ is recessed within the rotatable component, if the portable display unit 4000 is placed face-down on a surface, that will not actuate the push button D2 and will thereby inadvertent triggering or changing of settings of the breathing assistance apparatus.

In the configuration shown, the dial D projects from the upper surface 4013 of the display unit housing 4001. Alternatively, the dial D may be recessed in the display unit housing 4001.

A biasing device D3 such as a spring is provided between the push button D2 and a base D4 of the dial, to bias the push button D2 in the axial direction away from the display unit housing 4001. A user pushes the push button D2 in the axial direction towards the display unit housing to actuate the push button D2.

The base D4 of the dial may comprise a rotary encoder to convert the rotating of the rotatable component D1 to a digital signal. The base D4 may comprise a push button encoder which converts the push button D2 movement to a digital signal.

In an alternative configuration, the dial D may have a rotatable component D1 but no push button D2.

The portable display unit 4000 may comprise a proximity sensor 4101 to determine user presence.

The screen 4051 may be configured to dim or turn off if a user is not detected for a period of time, as sensed by the proximity sensor 4101.

The proximity sensor 4101 may, for example, comprise an optical sensor.

As outlined above, the portable display unit 4000 may further comprise an ambient light sensor 4102. The screen 4051 may be configured to adjust in brightness in response to ambient light changes.

The screen 4051 is recessed from the upper surface 4013 of the display unit housing 4001. That is, the display unit housing 4001 projects upwardly more than the screen 4051. That provides a level of impact protection for the screen—if the portable display unit 4000 is dropped or experiences an impact, there is unlikely to be a direct impact on the screen 4051.

As outlined above, the portable display unit may comprise at least one connection feature 4071 for removably connecting the portable display unit 4000 to the base unit 50 and to a different support component.

The at least one connection feature 4071 may comprise a first connection feature 4071″ for removably connecting the portable display unit 4000 to the base unit 50 and may comprise a second connection feature 4071′ for removably connecting the portable display unit 4000 to the different support component, wherein the first connection feature is different to the second connection feature.

As outlined above, in some configurations an angular orientation of the screen 4051 can be adjusted.

In some configurations, the angular orientation of the display unit housing 4001 can be adjusted relative to the at least one connection feature 4071 and/or relative to the base unit 50.

In some configurations, the portable display unit 4000 is hermetically sealed to prevent water and/or oxygen ingress into the portable display unit. In some configurations, a portable display unit 4000 for a breathing assistance apparatus is provided, the portable display unit comprising: a display unit housing 4001 defining an internal portion; a screen; and a tethering component 4061, TC1 for tethering the portable display unit with a base unit 50 of the breathing assistance apparatus; wherein the portable display unit 4000 is hermetically sealed to prevent water and/or oxygen ingress into the internal portion of the portable display unit.

The tethering component may comprise a cable 4061 for physically tethering the portable display unit 4000 with the base unit 50.

In some configurations, the cable 4061 is for communicating power to the portable display unit 4000 from the base unit 50 and/or for communicating data between the portable display unit 4000 and the base unit 50.

The tethering component may comprise a component of a wireless communication interface for communicating data between the portable display unit 4000 and the base unit 50.

As shown in FIG. 27 for example, the display unit housing 4001 comprises a first housing portion 4001a and a second housing portion 4001b, and a seal or gasket 4002 is provided between the first housing portion 4001a and the second housing portion 4001b.

The seal or gasket 4002 may comprise a compressible seal or compressible gasket, and the compressible seal or compressible gasket may be compressed between the first housing portion 4001a and the second housing portion 4001b in the assembled housing. The seal or gasket 4002 may comprise silicone or any other suitable material, including those described elsewhere herein for other seals or sealing functionality.

The seal or gasket 4002 may extend around the entire of the periphery of the display unit housing 4001 between the first housing portion 4001a and the second housing portion 4001b.

The portable display unit 4000 may comprise electrical and/or electronic components housed within the internal portion of the display unit housing 4001.

The electrical and/or electronics components may comprise a circuit board (PCB) 4004. The circuit board 4004 may comprise a printed circuit board (PCB).

The circuit board 4004 may be surrounded by a sealing material to prevent water and/or oxygen ingress into the circuit board.

In one exemplary configuration, the circuit board 4004 has the sealing material overmoulded onto the circuit board 4004.

The sealing material may be further configured to provide shock absorption and/or heat dissipation for the circuit board.

Suitable materials include those described elsewhere herein for shock absorption and heat dissipation functionality.

The portable display unit 4000 may further comprise a temperature sensor 4103 to determine if temperature in the internal portion of the portable display unit housing exceeds a threshold value. The temperature sensor 4103 may be provided on the circuit board 4004 or elsewhere.

The portable display unit 4000 may further comprise an oxygen sensor 4104 to determine if oxygen is detected in the internal portion of the portable display unit housing above a threshold value. The oxygen sensor 4104 may be provided on the circuit board 4004 or elsewhere.

The oxygen sensor 4104 may detect if oxygen is above a predetermined level, which may indicate a safety risk.

A seal or gasket 4155 such as an o-ring for example may be provided between the notification light 4150, 4150′ and the display unit housing 4001 to assist with hermetically sealing the display unit housing against water and/or oxygen ingress.

A seal or gasket E such as an o-ring for example may be provided between the button(s) B and the display unit housing 4001 and/or between the dial D and the display unit housing 4001 to assist with hermetically sealing the display unit housing against water and/or oxygen ingress.

The tethering component may provide a physical tether or a wireless tether of any of the types described herein.

As outlined above, the portable display unit 4000 may comprise at least one connection feature 4071 for removably connecting the portable display unit to the base unit 50 and to a different support component.

The at least one connection feature 4071 may comprise a first connection feature 4071″ for removably connecting the portable display unit 4000 to the base unit 50 and may comprise a second connection feature 4071′ for removably connecting the portable display unit 4000 to the different support component, wherein the first connection feature 4071″ is different to the second connection feature 4071′.

As outlined above, the screen 4051 may be recessed from an upper surface 4013 of the display unit housing. In some configurations, a portable display unit 4000 for a breathing assistance apparatus is provided, the portable display unit comprising: a display unit housing 4001 having an upper surface; a screen 4051; and a tethering component 4061, TC1 for tethering the portable display unit 4000 with a base unit 50 of the breathing assistance apparatus; wherein the screen 4051 is recessed from the upper surface 4103 of the display unit housing 4001.

An exemplary configuration is shown in FIG. 23.

The upper surface 4013 of the display unit housing 4001 may comprise an opening for the screen 4051. The screen 4051 may be at least partly located in and/or visible through, the opening in the upper surface 4013.

The display unit housing 4001 projects upwardly more than the screen 4051. That provides a level of impact protection for the screen 4051.

The material(s) of the display unit housing 4001 may be chosen to provide impact protection. Exemplary materials include polycarbonate, polycarbonate blends, or other plastics materials.

Due to the portable display unit 4000 having few or no moving parts, the material selection for the display unit housing 4001 may be a primary factor in resisting shock, impact, or drop damage.

The portable display unit 4000 may comprise features or functionality that indicate suitability for use following an impact or being dropped. For example, the portable display unit 4000 may be configured to withstand being dropped from a certain height, but the screen 4051 or a notification light may be configured to indicate whether an impact of more than a predetermined force has been experienced by the screen, indicating that a drop from a greater height has occurred.

The portable display unit 4000 may further comprise a sensor to detect if the portable display unit has been dropped or otherwise impacted. The sensor may comprise an accelerometer or a gyroscope for example. Additionally or alternatively, the sensor may detect the angle of orientation of the portable display unit 4000.

At least a portion of one or more corners and/or edges of the display unit housing 4001 may comprise or be integrally formed from an impact absorbent material, as described above for the corners 3511 of the display unit housing 3501.

The impact absorbent material may extend partly over the upper surface 4013 and a base 4009 of the display unit housing 4001.

In some configurations, the impact absorbent material is integrally formed with the display unit housing 4001. Alternatively, the impact absorbent material may be removable from the display unit housing 4001.

As outlined above, the display unit housing may define an internal portion, and the portable display unit may comprise electrical and/or electronic components housed within the internal portion of the display unit housing 4001.

The electrical and/or electronics components may comprise a circuit board 4004. The circuit board 4004 may comprise a printed circuit board (PCB).

The circuit board 4004 may be surrounded by a material that is configured to provide shock absorption, including those materials described elsewhere herein for shock absorption functionality. Additionally, or alternatively, the material may be a potting compound, such as a foam, or polystyrene.

In some configurations, the screen 4051 is held loosely or suspended in the display unit housing 4001. This provides for a level of shock protection for the screen 4051.

For example, the screen 4051 may be mounted in the display unit housing 4001 on resiliently deformable member(s). The member(s) may comprise bush(es) or suspension member(s). In one exemplary configuration, a suspension member may be provided at or adjacent each corner of the screen 4051 and may movably mount the screen 4051 to the display unit housing 4001.

As another example, the screen 4051 may be placed into the display unit housing 4001 and the screen 4051 may be clamped against the first housing portion 4001a. An impact absorbing material, such as a foam tape for example, are provided between the screen 4051 and the first housing portion 4001a.

The portable display unit may further comprise a sensor to detect if the portable display unit has been dropped. The sensor may comprise an accelerometer or a gyroscope for example. Additionally or alternatively, the sensor may detect the angle of orientation of the portable display unit 4000.

As outlined above, the portable display unit 4000 may comprise a notification light 4150, 4150′ and/or notification speaker S. The notification light 4150, 4150′ and/or notification sensor S may provide a visual and/or audible alert if the sensor detects that the portable display unit 4000 has been dropped or otherwise impacted.

The tethering component may provide a physical tether or a wireless tether of any of the types described herein.

As outlined above, the portable display unit 4000 may comprise at least one connection feature 4071 for removably connecting the portable display unit to the base unit 50 and to a different support component.

The at least one connection 4071 feature may comprise a first connection feature 4071″ for removably connecting the portable display unit 4000 to the base unit 50 and may comprise a second connection feature 4071′ for removably connecting the portable display unit 4000 to the different support component, wherein the first connection feature 4071″ is different to the second connection feature 4071′.

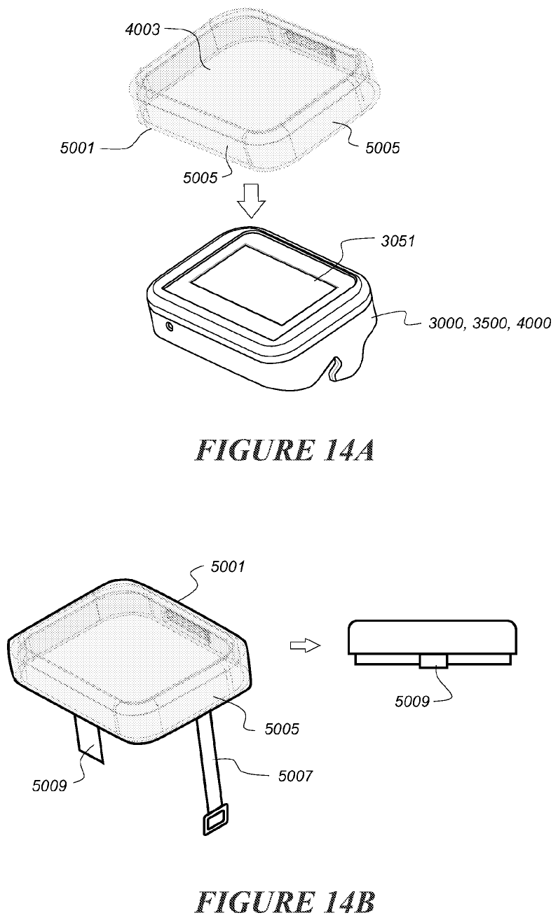

The portable display unit 3000, 3500, 4000 may be provided with a screen cover 5001. An exemplary screen cover is shown in FIGS. 14A and 14B.

The screen cover 5001 comprises a transparent flexible material, and is configured to cover at least the screen 3051, 3551, 4051 of the portable display unit 3000, 3500, 4000.

In the configuration shown, the screen cover 5001 comprises an upper surface 5003 for covering the screen 3051, 3551, 4051 and a plurality of walls 5005 for extending around the walls 3003, 3005, 3007, 3503, 3505, 3507, 4003, 4005, 4007 of the display unit housing 3001, 3501. The screen cover 5001 may also be configured to extend partly around the base 3009, 3509, 4009 of the display unit housing 3001, 3501, 4001.

The screen cover 5001 preferably comprises a resilient material and may be an interference fit so that the screen cover 5001 needs to be stretched to mount the cover onto the display unit housing 3001, 3501, 4001. That will assist with retaining the screen cover 5001 in place on the display unit housing 3001, 3501, 4501.

Additionally, or alternatively, the screen cover 5001 may comprise securing feature(s) to assist with retaining the screen in place on the display unit housing 3001, 3501, 4001.

In the form shown, exemplary securing feature(s) comprise an elongate strap 5007 extending from one side of the cover 5001 and that is configured to couple to a connector 5009 extending from another side of the screen cover, so that the strap 5007 extends at least partly across the base of the display unit housing 3001, 3501, 4001. Any other suitable configuration could be used. The strap 5007 and/or connector 5009 may comprise a buckle, hook and loop fasteners, or any other suitable coupler.

When the screen cover 5001 is in position on the portable display unit 3000, 3500, 4000, an air gap may be provided between the cover 5001 and the screen 3051, 3551, 4051. That will enable the touch screen to still be used if liquid contacts the cover 5001.

The air gap may additionally be provided between the cover 5001 and the display unit housing 3001, 3501, 4001 to provide insulation from heat.

The screen cover 5001 may be a removable screen cover. Alternatively, the screen cover 5001 may be overmoulded onto the screen. When an overmoulded screen cover is used, the screen cover will not have securing feature(s).

When the screen cover 5001 is a removable screen cover, the screen cover 5004 may be removed from the portable display unit 3000, 3500, 4000 and cleaned, sterilised, or disposed of, and may be replaced by a new cover 5001.

The screen cover 5001 may help prevent false touches on the screen 3051, 3551, 4051, and may help prevent water ingress into the portable display unit 3000, 3500, 4000.

In some configurations, the screen cover 5001 may be sized and configured only cover the screen or the screen and the upper surface of the housing, rather than other parts of the housing.

Material selection for the screen cover 5001 may involve semi-rigid material which is tough enough to withstand impact. That is, the screen cover 5001 may comprise a semi-rigid material which is tough enough to withstand impact. This may be a singular piece with increased thickness at the corners of the screen cover corresponding to the corners of the portable display unit. The material will advantageously be thin enough, and transparent, to enable actuation/use of the touchscreen.

Exemplary materials may include silicone or polyurethane. Other foam materials may be used to provide impact absorption.

When an overmoulded screen cover 5001 is provided, the screen cover 5001 may assist with preventing water and/or oxygen ingress into the portable display unit 3000, 3500, 4000.

The screen cover 5001 may provide heat sinking or heat transfer capabilities to enable the transfer of heat away from the portable display unit 3000, 3500, 4000 through thermal property selection of the material of the screen cover 5001.

Exemplary materials for the screen cover include silicone, thermally conductive silicone rubber, or any other suitable materials.

The screen cover 5001 may provide for enhanced grip of the portable display unit 3000, 3500, 4000.

The portable display unit 3000, 3500, 4000 may be provided in combination with the breathing assistance apparatus, or may be provided as an aftermarket device for use with the breathing assistance apparatus.

The portable display unit may be used with any of the types of breathing assistance apparatuses disclosed herein. The breathing assistance apparatus may, for example, be a respiratory assistance apparatus, respiratory therapy device, or any other suitable type of breathing assistance apparatus.

Any of the breathing assistance apparatuses disclosed herein may comprise complementary connection feature(s) for engaging with the connection feature(s) of the portable display unit 3000, 3500, 4000, to removably connect the display unit to the breathing assistance apparatus.

The portable display unit 3000, 3500, 4000 may be connectable to and separable from the base unit 50 of the breathing assistance apparatus.

The portable display unit 3000, 3500, 4000 may be tethered with the base unit 50. The portable display unit 3000, 3500, 4000 may be physically tethered with the base unit 50 and/or may be wirelessly tethered with the base unit 50 for communicating data between the portable display unit and the base unit.

In at least one exemplary configuration, a breathing assistance apparatus comprises: a humidifier 12, 1302a, 1302b, 300; a base unit 50; and a portable display unit 3000, 3500, 4000 comprising: a display unit housing 3001, 3501, 4001; a screen 3051, 3551, 4051; a communication interface for communicating data and/or control signals between the base unit 50 and the portable display unit 3000, 3500, 4000; and at least one connection feature 3071, 3571, 4071 for removably connecting the portable display unit to the base unit 50.

The humidifier may be provided in the base unit.

The communication interface may comprise at least one wire for communicating data and/or control signals between the portable display unit and the base unit.

The communication interface may comprise a wireless communication interface for communicating data and/or control signals between the portable display unit and the base unit.

In at least one exemplary configuration, a breathing assistance apparatus comprises: a base unit 50; a heating element 140 positioned on or within the base unit 50; a humidification chamber 300 associated with the heating element 140, such that the heating element is in thermal communication with the humidification chamber; a controller 13 in electronic communication with the heating element 140; and a display unit 3000, 3500, 4000, wherein the display unit 3000, 3500, 4000 is separable from the base unit 50, such that the display unit 3000, 3500, 4000 can be separated from the base unit and reconnected to the base unit 50, wherein the controller 13 is in electronic communication with the display unit 3000, 3500 when the display unit is connected to and separated from the base unit 50, such that data and/or control signals and/or power signals can be transmitted from the controller to the display unit and data and/or control signals can be transmitted from the display unit to the controller.

The breathing assistance apparatus may be configured to receive gases from an external source.

Alternatively, the breathing assistance apparatus may comprise a flow generator (e.g. a blower or motor module) in the base unit to generate a flow of pressurised gases.

The humidification chamber 300 may be removable from the base unit.

The display unit 3000, 3500, 4000 may be physically tethered to the base unit 50 to enable transmission of power signals from the controller to the display unit. The physical tethering may be by any of the configurations disclosed herein.

The display unit 3000, 3500, 4000 may be physically tethered to the base unit 50 to enable transmission of data from the controller to the display unit and to enable transmission of data from the display unit to the controller. The physical tethering may be by any of the configurations disclosed herein.

The display unit 3000, 3500, 4000 may be wirelessly tethered to the base unit 50 to enable transmission of data from the controller to the display unit and to enable transmission of data from the display unit to the controller. The wireless tethering may be by any of the configurations disclosed herein.

The display unit 3000, 3500, 4000 disclosed herein may be used as part of a modular breathing assistance apparatus. The breathing assistance apparatus may have a plurality of components comprising: a flow generator 11, 1301a, 1301b, 1307, 1726, 2125 to generate a flow of pressurised gases; a humidifier 12, 300, 1302a, 1302b, 2102, 2104 to humidify the gases; and a display unit 3000, 3500, 4000 including a display unit housing 3001, 3501, 4000, a screen 3051, 3551, 4051, and a tethering component (TC1, TC2 and/or TC3) for tethering the display unit 3000, 3500, 4000 with at least one of the components of the breathing assistance apparatus, wherein the components are modular such that at least one of the components is separable from at least one other of the components.

In some configurations, the display unit 3000, 3500, 4000 is separable from at least one other component of the breathing assistance apparatus.

In some configurations, the humidifier 12, 300, 1302a, 2102, 2104 and flow generator 11, 1301a, 1307, 1726, 2125 are separable from each other.

In some configurations, the humidifier 12, 1302a is in a humidifier housing, the flow generator 11, 1301a is in a separate flow generator housing, and the humidifier housing can be removably coupled to the flow generator housing.

In some configurations, the display unit 3000, 3500, 4000 is separable and removably coupled to the flow generator housing.

In some configurations, the display unit 3000, 3500, 4000 is separable and removably coupled to the humidifier housing.

In some configurations, the humidifier comprises a first display unit 3000, 3500, 4000, the flow generator comprises a second display unit 3000, 3500, 4000, and both display units are removable and portable.

In some configurations, the humidifier 300, 1302b and the flow generator 11, 1301b are in a common housing.

In some configurations, the humidifier comprises a heater plate 140 that is attached to the common housing and a humidification chamber 300 that can be removably positioned within a portion of the common housing such that the heater plate is in thermal communication with the heater plate. In some configurations, the heater plate 140 contacts the humidification chamber 300 when the humification chamber is in the common housing.

The display unit 3000, 3500, 4000 may be separable and removably couplable to the common housing.

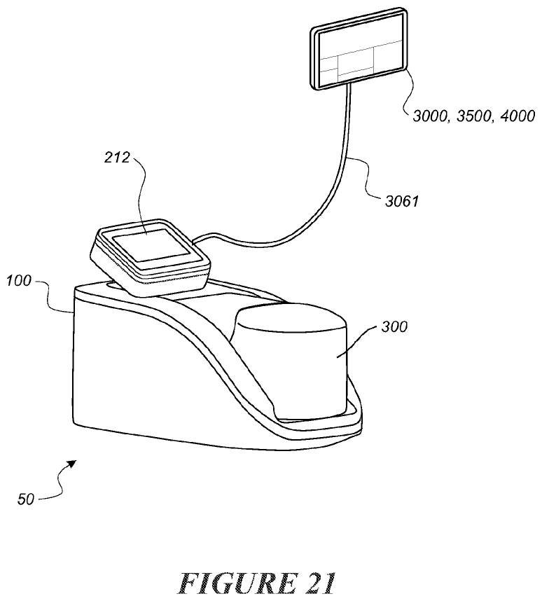

Any of the breathing assistance apparatuses described herein may comprise more than one display. As shown in FIG. 21 for example, a base unit 50 of an exemplary breathing assistance apparatus may have a first display screen 212 fixed to the housing 100 of the base unit. The portable display unit 3000, 3500, 4000 may also be provided for use with that breathing assistance apparatus. When the portable display unit 3000, 3500, 4000 is provided, that portable display unit may act as the primary screen for the breathing assistance apparatus with the display screen 212 acting as a secondary or backup screen. That is, the portable display unit 3000, 3500, 4000 may be the primary interface for the breathing assistance apparatus, and he portable display unit 3000, 3500, 4000 may be the secondary interface for the breathing assistance apparatus. In alternative configurations, the portable display unit may act as the primary screen for the breathing assistance apparatus with the display screen 212 also acting as the primary screen. In these configurations, the portable display unit and the display screen 212 may mirror each other, or otherwise allow the same functionality relating to monitoring of therapy and adjustment of therapy parameters.

In an alternative configuration, the display screen 212 may also be removable from the base unit and portable.

By providing at least one portable display unit 3000, 3500, 4000, it is possible to provide a screen for use with a breathing assistance apparatus that enables optimal placement of the screen for use (e.g. at or near eye level of the patient, user, or medical personnel), while enabling the breathing assistance apparatus base unit to be positioned elsewhere.

In at least some exemplary configurations, a portable display unit 3000, 3500, 4000 for a breathing assistance apparatus is disclosed, wherein the portable display unit can be, or is configured to be, positioned in an operative position that is separate from a base unit of the breathing assistance apparatus and vertically higher than the base unit.

For example, when the breathing assistance apparatus comprises an integrated humidifier or is a humidifier, the humidifier can be positioned below the patient while the portable display unit 3000, 3500, 4000 can be, or is configured to be, positioned in the operative position at a higher location that vertically higher than/above the base unit 50 and humidifier that is readily viewable/usable. This enables user interaction and control of the breathing assistance apparatus from an advantageous position.

The base unit 50 may comprise an integrated flow generator/blower and an integrated humidifier.

In some configurations, the breathing assistance apparatus further comprises a breathing circuit in fluid communication with the humidifier and the breathing circuit comprising a patient interface. The humidifier can be, or is configured to be, placed below patient head height. The breathing circuit and humidifier are configured such that any condensate that accumulates in the breathing circuit will be encouraged to return to the humidifier rather than collecting at the patient interface. Gravity will assist with returning the condensate to the humidifier. Additionally, if the humidifier humidification chamber is overfilled, the overfilled liquid should not collect at the patient interface.

In some configurations, the portable display unit 3000, 3500, 4000 can be positioned above the base unit 50 and the humidifier.

In some configurations, when the portable display unit 3000, 3500, 4000 is above the base unit 50 and/or the humidifier, the portable display unit enables user interaction and control of the breathing assistance apparatus from an advantageous position.

In some configurations, the humidifier may be, or is configured to be, placed under patient hip height in order to let condensate drain into the humidification chamber.

The humidifier or high flow apparatus, at least in hospital uses, are positioned below the patient's bed or clinician's hip due to the volume of water and the heater plate. This is for safety of the clinician and patient. This reduces the chance of spilling heated and/or contaminated water on the patient. However this means an integrated screen is out of visibility of the clinician. The portable display unit allows a clinician to detach the display unit and position it in a more visible position to allow a clinician to view the display from a convenient position while still keeping the base unit at a safe position.

In at least some exemplary configurations, a method of using a portable display unit 3000, 3500, 4000 of a breathing assistance apparatus is disclosed, the breathing assistance apparatus comprising a base unit 50 including a humidifier, the portable display unit 3000, 3500, 4000 being tethered to the base unit, the method comprising the steps of: disconnecting the portable display unit 3000, 3500, 4000 from the base unit, and positioning the portable display unit 3000, 3500, 4000 above the base unit 50 such that the portable display unit 3000, 3500, 4000 is visible to a user.

In some configurations, the method comprises extending the tether or manipulating the tether to position the portable display unit in a location that is at eye level of the user or adjacent a patient.

In some configurations, the breathing assistance apparatus comprises a humidifier.

In some configurations, the method comprises positioning the humidifier below patient head height and positioning the portable display unit 3000, 3500, 4000 in the operative position so as to be readily viewable/usable.

In some configurations, the breathing assistance apparatus further comprises a breathing circuit in fluid communication with the humidifier and the breathing circuit comprises a patient interface. When the when the humidifier is placed below patient head height, any condensate which accumulates in the breathing circuit is encouraged to return to the humidifier rather than collecting at the patient interface.

In some configurations, the method comprises placing the humidifier under patient hip height in order to let condensate drain into a humidification chamber of the humidifier.

In some configurations, the method comprises placing the portable display unit 3000, 3500, 4000 above the base unit and the humidifier.

In some configurations, when the portable display unit 3000 is above the base unit and/or the humidifier, the display unit enables user interaction and control of the breathing assistance apparatus from an advantageous position.

When provided, the support features 3071, 3571, 4071 of the portable display units 3000, 3500, 4000 enable the display units to be supported on and connected to a variety of different support surfaces or different support components. The support surfaces or support components will be separate from the base unit 50.

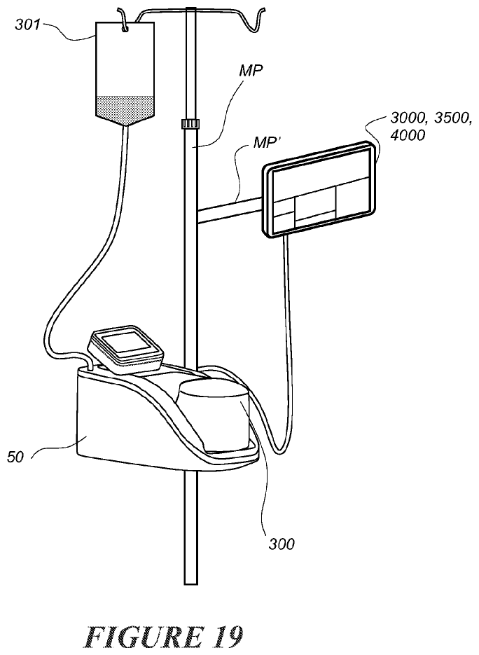

FIG. 19 shows the base unit 50 of the breathing assistance apparatus connected or mounted to a support component comprising medical pole MP with a liquid bag 301 delivering liquid to the humidifier humidification chamber 300. The portable display unit 3000, 3500, 4000 can be supported on a transverse member MP′ that extends from the medical pole, or can be supported on another support surface. The medical pole MP may be provided with one or more complementary connector features 2071, 2071′ for engagement with the connector feature 3071, 3571, 4071 on the portable display unit 3000, 3500, 4000.

As another example, FIG. 18A shows the portable display unit 3500 hanging on the end wall EW of a support component comprising a cot or bed whereas FIG. 18B shows the portable display unit 3500 supported on a side wall SW of a support component comprising a bench and FIG. 18C shows the portable display unit 3500 supported on a support component comprising a substantially horizontal support surface. The cot or bed, bench, or horizontal support surface may be provided with one or more complementary connector features 2071, 2071′ for engagement with the connector feature 3071, 3571, 4071 on the portable display unit 3000, 3500, 4000.

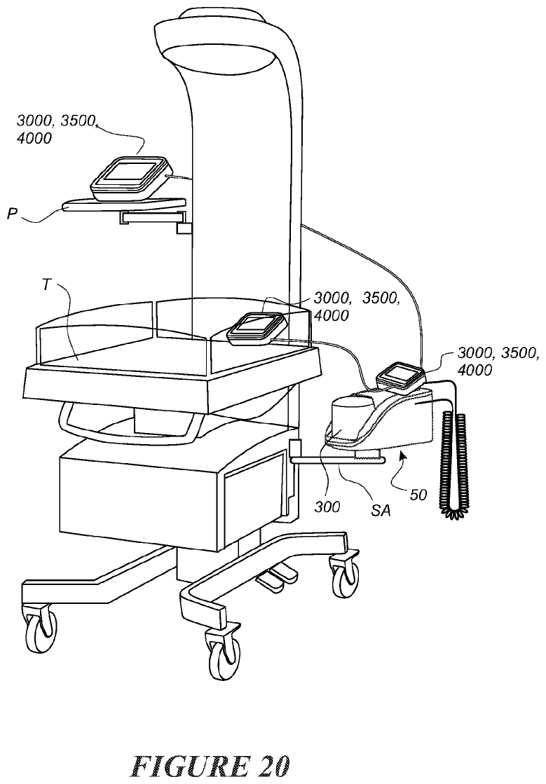

As another example, FIG. 20 shows exemplary support positions for the portable display unit 3000, 3500, 4000 on a support component comprising a medical stand, including supporting the portable display unit 3000, 3500, 4000 on a platform P, on a table, or having the base unit mounted to a support arm and the portable display unit 3000, 3500, 4000 connected to the base unit 50. Similar options are available for a warmer, an incubator, a bed mount, or other medical apparatuses. The medical stand may be provided with one or more complementary connector features 2071, 2071′ for engagement with the connector feature 3071, 3571, 4071 on the portable display unit 3000, 3500, 4000.

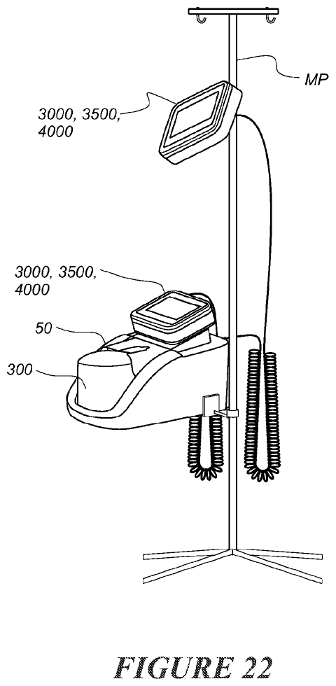

As another example, FIG. 22 shows the portable display unit 3000, 3500, 4000 either connected to the base unit 50 of the breathing assistance apparatus that is mounted to the medical pole MP, or connected to or supported on a transverse member that extends from the medical pole. The medical pole may be provided with one or more complementary connector features 2071, 2071′ for engagement with the connector feature 3071, 3571, 4071 on the portable display unit 3000, 3500, 4000.

The portable display unit 3000, 3500, 4000 may be angularly adjustable, rotatable, height adjustable, and/or laterally selectively moveable when mounted to said support component. For example, the complementary connector feature(s) 2071, 2071′ may be selectively moveable or adjustable relative to the support component, optionally while remaining connected to the support component.

Additionally or alternatively the portable display unit 3000, 3500, 4000 may be connected to a portion of the support component that is selectively moveable relative to another portion of the support component. For example, the portion of the support component may comprise an adjustable arm.

The portable display unit 4000 may be tethered to the base unit 50 by a cable. In some configurations a breathing assistance apparatus is provided, the breathing assistance apparatus comprising: a base unit 50; a portable display unit 4000 comprising: a display unit housing 4001; and a screen 4051; and a cable 4061 physically tethering the portable display unit 4000 with the base unit 50, wherein the cable 4061 is for communicating power to the portable display unit 4000 from the base unit 50 and/or for communicating data between the portable display unit 4000 and the base unit 50, wherein the cable 4061 is connected to the portable display unit 4000 by an overmoulded portion 4200 of the cable which removably connects to a portion of the display unit housing 4001 and/or is connected to the base unit 50 by a retention portion 4300 of the cable which removably connects to a portion of the base unit 50.

An exemplary configuration is shown in FIGS. 29 and 38-42.

In some configurations, the cable 4061 is connected to the portable display unit 4000 by the overmoulded portion 4200. In some configurations, the cable 4061 is connected to the base unit 50 by the retention portion 4300. In some configurations, the cable is both connected to the portable display unit 4000 by the overmoulded portion 4200 and to the base unit 50 by the retention portion 4300.

A shape of the overmoulded portion 4200 of the cable conforms or corresponds to a shape of a slot 4008 in the portion of the display unit housing 4001.

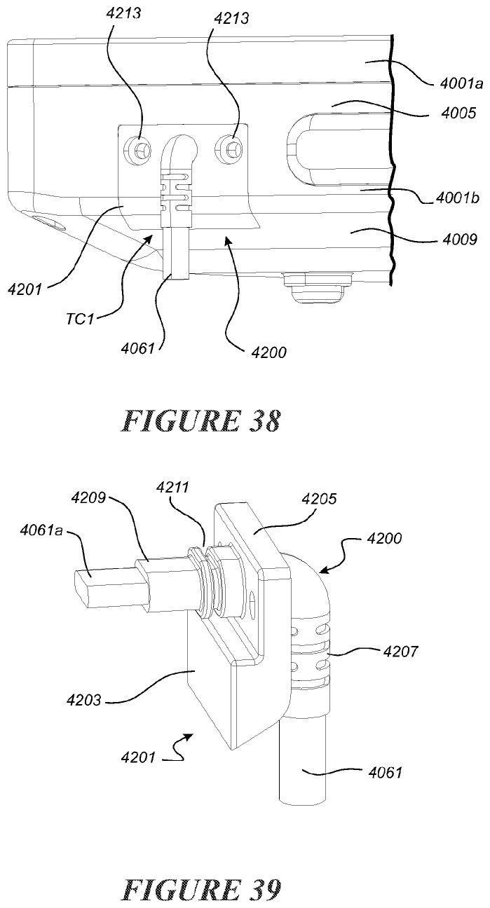

FIGS. 38-40 show exemplary configurations of the overmoulded portion 4200 and the slot 4008.

The shape of the overmoulded portion 4200 of the cable and the slot 4008 are such that the overmoulded portion 4200 of the cable, and thereby the cable 4061, can only be inserted into the slot 4008 in the portion of the display unit housing 4001 in a single orientation. The cable 4061 may only be intended for use in a certain orientation. As such, the overmoulded portion 4200 of the cable may act as polarisation feature so that a connector 4061a on the cable can only be inserted or plugged into the display unit housing 4001 in a single orientation.

The overmoulded portion 4200 of the cable comprises a shaped body portion 4201. The body portion has a shape that conforms or corresponds to the shape of the slot 4008.

In the exemplary configuration shown, the body portion 4201 comprises a base portion 4203 and an adjacent portion 4205. The base portion 4203 is proximal to the elongate portion of the cable that extends away from the display unit housing 4001 toward the base unit 50. The adjacent portion is proximal to the connector 4061a.

In the exemplary configuration shown, the slot 4008 comprises a complementary base portion 4008a and a complementary adjacent portion 4008b.

The body portion 4201 further comprises a cable sheath portion 4207 through which the cable passes.

The connector 4061a protrudes from a neck portion 4209 of the overmoulded portion 4200.

The overmoulded portion 4200 of the cable may comprise a groove 4211 for receipt of a seal or grommet to assist hermetically sealing against water and oxygen ingress into the display unit housing 4001. The seal may comprise an o-ring. The seal will seal against an aperture 4008c in the slot.

In an alternative configuration, a seal or grommet may be integrally moulded to the neck portion 4209 of the overmoulded portion 4200, in a similar position to that shown for the groove 4211.

The body portion 4201 is shaped to conform to the shape of the slot 4008 in the portion of the display unit housing. In the configuration shown, the base portion 4203 is thicker than the adjacent portion 4205. Other configurations are possible.

In the configuration shown, the base portion 4008a of the slot is deeper than the adjacent portion 4008b of the slot.

The outer surface(s) of the overmoulded portion 4200 of the cable are advantageously shaped so that the overmoulded portion 4200 conforms to the external shape of the display unit housing 4001 adjacent to the slot 4008. The outer surface(s) may sit flush with the display unit housing adjacent the slot 4008.

As shown the cable 4061 extends through the overmoulded portion 4200 of the cable such that the connector 4061a is oriented at a transverse angle relative to the cable 4061. In an alternative configuration, the overmoulded portion 4200 of the cable may be configured such that the connector 4061a is aligned with the cable 4061.

The connector 4061a of the cable 4061 extends through the aperture 4008c when the overmoulded portion 4200 of the cable is inserted in the slot 4008.

The overmoulded portion 4200 of the cable is configured to be fastened to the display unit housing 4001 by one or more fasteners to inhibit removal of the cable 4061 from the portable display unit 4000. In the configuration shown, the body portion 4201 comprises one or more fastener receiving apertures 4213 for receipt of fasteners such as screws or the like. The fastener(s) extend through the fastener receiving aperture(s) 4213 and are received in complementary aperture(s) 4008d in the slot 4008 of the display unit housing 4001.

In an alternative configuration, the overmoulded portion 4200 of the cable may be configured to be fastened to the display unit housing 4001 by one or more integral clip features, adhesive, or any other suitable technique.

By fastening the overmoulded portion 4200 of the cable to the display unit housing 4001, the connector 4061a is inhibited from being pulled out of the display unit housing or damaged if the cable 4061 is snagged.

The overmoulded 4200 of the cable provides a hermetic seal against water and oxygen ingress at the connection of the cable 4061 to the portable display unit 4000.

The cable 4061 may be removable from the portable display unit 4000 by removing the fastener(s).

In some configurations, the cable 4061 is connected to the base unit 50 by the retention portion 4300.

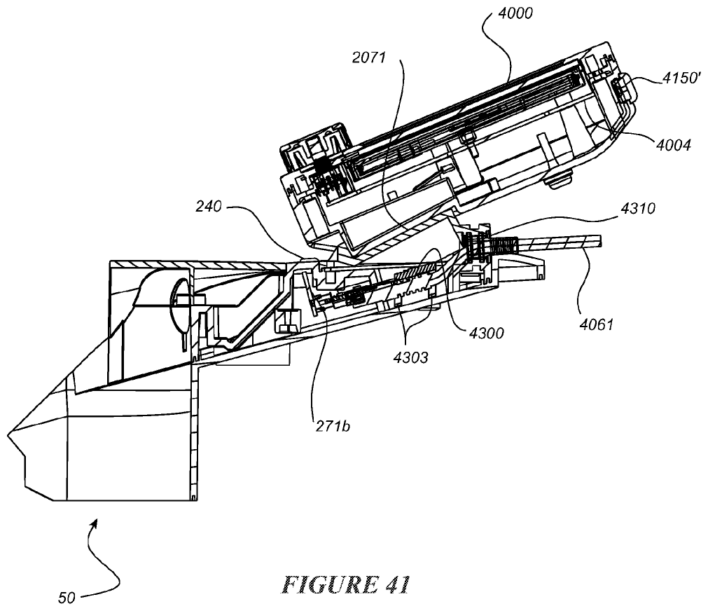

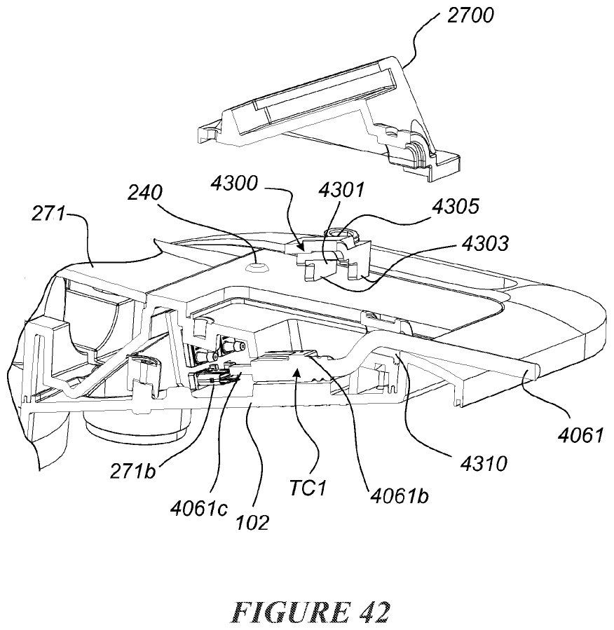

An exemplary configuration is shown in FIGS. 41 and 42.

A shape of the retention portion 4300 of the cable conforms or corresponds to a shape of a slot in the base unit 50. In the exemplary configuration shown in FIG. 42, the retention portion 4300 of the cable comprises a body portion 4301 with one or more projections 4303 configured to be received in the slot in the base unit 50.

The retention portion 4300 of the cable may also be configured to be received in a slot 4061b in the cable 4061.

The retention portion 4300 may be a separate component from the cable 4061, or may be integrally formed with the cable 4061. The retention portion 4300 may be an overmoulded portion of the cable 4061.

The retention portion 4300 of the cable is configured to be fastened to the base unit 50 by one or more fasteners to inhibit removal of the cable 4061 from the base unit 50. In the configuration shown, the body portion 4301 comprises one or more fastener receiving apertures 4305 for receipt of fasteners such as screws or the like. The fastener(s) extend through the fastener receiving aperture(s) 4305 and is/are received in complementary aperture(s) in the base unit 50.

In an alternative configuration, the retention portion 4300 of the cable may be configured to be fastened to the base unit 50 by one or more integral clip features.

In an alternative configuration, the retention portion 4300 may be configured to be fastened to the base unit 50 by way of the display unit mounting 2072 to the base unit 50. The fastening of the display unit mounting 2072 to the base unit may prevent the cable from being removed from the base unit 50.

By fastening the retention portion 4300 of the cable to the base unit 50, the connector 4061c is inhibited from being pulled out of the base unit 50 or damaged if the cable 4061 is snagged.

The cable 4061 may be removable from the base unit 50 by removing the fastener(s).

The shape of the retention portion 4300 of the cable may be such that the cable 4061 can only be inserted into the base unit 50 in a single orientation. In some configurations, the shape of the retention portion 4300 of the cable and the cable 4061 are such that the cable 4061 can only be inserted into the base unit 50 in a single orientation. For example, the retention portion 4300 may be configured to only fit with one side of the cable, so that if the cable is inverted the retention portion cannot engage with the cable. Additionally or alternatively, the retention portion 4300 of the cable, the cable 4061, and/or the base unit 50 may have one or more other features to ensure the cable can only be inserted into the base unit on a single orientation. As such, the retention portion of the cable may act as polarisation feature so that a connector 4061c on the cable can only be inserted or plug into the cable connector 271b on the interconnect circuit board 271 in a single orientation.

The breathing assistance apparatus may further comprise a sealing feature 4310 that provides a hermetic seal against water and/or oxygen ingress at the connection of the cable 4061 to the base unit 50.

In some configurations, the sealing feature 4310 comprises a seal or a gasket. In the configuration shown, the sealing feature 4310 comprises a grommet.

The grommet may be inserted onto the cable 4061 or may be integrally moulded or otherwise pre-assembled onto the cable 4061.

The grommet seals against a portion of the housing of the base unit 50.

The grommet may comprise silicone or any other suitable material.

The sealing feature 4310 may be integral with the retention portion 4300, separate from the retention portion 4300, or both integral with the retention portion 4300 and separate from the retention portion 4300 (i.e. may have a portion that is integrally formed with the retention portion 4300 and may have a portion that is separate from the retention portion 4300).

The cable 4061 may be configured to be connected to the upper housing chassis 102 or alternatively to a different part of the housing 100 of the base unit 50.

As outlined above, the cable may have a length of up to about 2 m, optionally of between about 1.5 m and about 2 m.

The cable 4061 can be replaceable on-site by a service technician if the display unit 4000 or cable 4061 gets damaged.

If the cable 3061, 3561, 4061 is separable from or provided separately from the portable display unit 3000, 3500, 4000, the portable display unit tethering component TC1 may comprise a connector for connecting the cable 3061, 3561, 4061 to the portable display unit 3000, 3500, 4000.

If the cable 3061, 3561, 4061 is separable from, or provided separately from, the base unit 50, the base unit 50 may comprise a connector for connecting the cable 3061, 3561, 4061 to the base unit 50.

The breathing assistance apparatus may comprise at least one security feature to only allow connection of a genuine portable display unit 3000, 3500, 4000 to the base unit 50.

The at least one security feature may be provided at least partly by the connection of the cable 4061 to the portable display unit 4000 and/or at the connection of the cable 4061 to the base unit 50.

The at least one security feature comprises a hardware and/or software module to determine whether the connected portable display unit 4000 is genuine.

The at least one security feature may include a separate security chip (i.e. a separate hardware module) or may be integrated into the interconnect PCB 271, or base unit main base unit circuit board 272.

The at least one security feature may comprise a software module that checks the incoming connection to the portable display unit 4000 and is able to determine if the portable display unit 4000 is allowed for use (i.e. is not a third party portable display unit). If the portable display unit 4000 is not legitimate, then the base unit 50 may not allow data and power connections to the portable display unit 4000, and/or may alert the user, or may alert the manufacturer or supplier.

The alert to the user may be through the display unit notification light 4150, 4150′, the base unit notification light 240, 240′, and/or the speaker S.

Additionally, or alternatively, the connection may be encrypted, such that the base unit 50 will only work with cables 4061 and/or portable display units 4000 which have corresponding encryption. The portable display unit 4000 may have an encryption key that enables access to portable display unit or vice versa. This similarly prevents the use of third party screen modules.

In one exemplary configuration one of the base unit 50, cable 4061, or portable display unit 4000 has a chip or integrated circuit that issues a series of numbers, and another one of the base unit 50, cable, or portable display unit 4061 has a chip or integrated circuit that checks the numbers for a match.

In some configurations, the hardware and/or software module is provided by a security circuit.

In some configurations, the security circuit comprises one or more encryption modules which allow an encrypted connection to be established through the cable if the portable display unit is genuine.

The base unit 50 may comprise a cable management arrangement to enable the cable 3061, 3561, 4061 to be coiled. The cable management arrangement may further allow other cables to be coiled, such as a power cable for the base unit, cables for one or more sensors, etc.

The cable management arrangement may comprise a cable hanger. The cable hanger enables the cable to be hung from the cable hanger. The cable hanger allows the cable 4061 to be coiled about the cable hanger.

The cable hanger projects from a portion of the housing 100 of the base unit.

In an advantageous configuration, the cable hanger projects from a portion of the housing 100 so that the cable hanger and the cable 4061 do not interfere with access to the humidification chamber bay 108 or portable display unit 4000 when the portable display unit 4000 is connected to the base unit 50.

In some configurations, the cable hanger projects from a side and/or the rear of the base unit housing 100.

The cable hanger may comprise a main support portion 4401 and a retainer portion 4403 to assist with retaining the cable 4061 on the main support portion 4401. The retainer portion 4403 may be an enlarged portion having at least one dimension larger than the main support portion 4401. In one configuration, the retainer portion 4403 comprises a flange.

As outlined above, the cable 4061 may be retractable. For example, the cable 4061 may be retractable at the portable display unit 4000 or at the base unit 50.

Any of the portable display units 3000, 3500, 4000 may have direct communications capability, i.e. not to or from the base unit 50. The direct communications capability may be wired or wireless, and may be any of the types described herein. The direct communications enable communications to and/or from the portable display unit 3000, 3500, 4000 directly, without transmitting back to the base unit 50 first.

For example, the direct communications capability might be via a nurse alarm cable that plugs into a hospital nurse alarm port, or the portable display unit may have Wi-Fi and/or a Bluetooth integrated circuit and/or a cellular communications integrated circuit for connecting to an external device. Exemplary external devices include a smartphone, laptop, tablet, desktop computer, or other medical devices.

The portable display units 3000, 3500, 4000 described herein may be used while connected to the base units 50 or while disconnected from the base units 50. In some configurations, the portable display units may be used while connected to the different support component, e.g. a medical pole or medical stand, but may be connected to the base unit for use during transport to, from, or around a medical facility or when not being used for monitoring a patient.

Although the present disclosure has been described in terms of certain embodiments, other embodiments apparent to those of ordinary skill in the art also are within the scope of this disclosure. Thus, various changes and modifications may be made without departing from the spirit and scope of the disclosure. For instance, various components may be repositioned as desired. Features from any of the described embodiments may be combined with each other and/or an apparatus may comprise one, more, or all of the features of the above described embodiments. Moreover, not all of the features, aspects and advantages are necessarily required to practice the present disclosure. Accordingly, the scope of the present disclosure is intended to be defined only by the claims that follow.

The various configurations described are exemplary configurations only. Any one or more features from any of the configurations may be used in combination with any one or more features from any of the other configurations.

The features are described with reference to a breathing assistance apparatus that can deliver heated and humidified gases to a patient or user. The apparatus may be suitable for treating chronic obstructive pulmonary disease (COPD). The apparatus may be configured to deliver gases to a patient interface at a high flow rate (high flow therapy), particularly nasal high flow therapy.

Alternatively, the features may be used with an apparatus for a different purpose. The apparatus may be a high flow therapy apparatus, or may be a low flow therapy apparatus. For example, the features may be provided in an apparatus for providing continuous positive airway pressure (CPAP), which may deliver gases (humidified or otherwise) at lower flow rates, or may be provided in a medical insufflation apparatus.

CPAP is primarily for out-of-hospital use e.g. home use. High flow is for both hospital and out-of-hospital use. Humidifiers are primarily for hospital use, but may also be used in out-of-hospital care.

The features may be used with a nasal high flow (NHF) apparatus.

The features could be used with a stand-alone humidifier. The standalone humidifier may have a housing, a recess 108 for receipt of the humidification chamber 300, and a heater plate 140, but may not have a motor unit. The standalone humidifier may receive gases from an external source.

Accordingly, an alternative form breathing assistance apparatus 10 may be a standalone humidifier apparatus comprising a base unit 50 defining a main housing and a humidifier 12.

The standalone humidifier apparatus can deliver heated and humidified gases for various medical procedures, including respiratory therapy, laparoscopy, and the like. These apparatuses can be configured to control temperature and/or humidity. The apparatuses can also include medical circuits comprising various components that can be used to transport heated and/or humidified gases to and/or from patients. For example, in some breathing circuits, gases inhaled by a patient are delivered from a heater-humidifier through an inspiratory tube or conduit. As another example, tubes can deliver humidified gas (commonly CO₂) into the abdominal cavity in insufflation circuits. This can help prevent desiccation or ‘drying out’ of the patient's internal organs, and can decrease the amount of time needed for recovery from surgery. Heater wires may extend inside of at least a portion of the tubing forming the circuit to prevent or at least reduce the likelihood of the formation of significant condensation.

A standalone humidifier apparatus would typically include a base unit 50 and a humidification chamber 300. The base unit 50 can comprise a heater plate 140. The humidification chamber 300 can be configured to hold a volume of a liquid, such as water. The heater plate can be configured to heat the volume of liquid held within the humidification chamber 300 to produce vapour.

The humidification chamber 300 is removable from the base unit to allow the humidification chamber to be more readily sterilized or disposed, or to re-fill the chamber with liquid. The body of the humidification chamber 300 can be formed from a non-conductive glass or plastics material but the humidification chamber can also include conductive components. For instance, the humidification chamber can include a highly heat-conductive base (for example, an aluminum base) contacting or associated with the heater plate on the heater base.

The base unit can also include electronic controls such as a master controller. In response to user-set humidity or temperature values input via a user interface and other inputs, the master controller determines when (or to what level) to energize the heater plate 140 to heat the liquid within the humidification chamber 300.

The standalone humidifier apparatus can include a flow generator to deliver gases to the humidification chamber. In some configurations, the flow generator can comprise a ventilator, blower, or any other suitable source of pressurized gases suitable for breathing or use in medical procedures. The flow generator may be positioned in the base unit 50.

Alternatively, the standalone humidifier apparatus may comprise just the base unit 50 and the humidification chamber 300, and may be used with a separate or remote flow generator. The base unit 50 may be configured to fluidly connect to the separate or remote flow generator.

Therefore, the flow generator that is used with a standalone humidifier apparatus may be a wall gases source, ventilator, blower, or gas tank for example.

A standalone humidifier apparatus can be used with breathing therapies, positive pressure apparatus, noninvasive ventilation, surgical procedures including but not limited to laparoscopy, and the like. Desirably, the humidifier apparatus can be adapted to supply humidity or vapour to a supply of gases. The humidifier apparatus can be used with continuous, variable, or bi-level PAP systems or other form of respiratory therapy. In some configurations, the humidifier apparatus can be integrated into a system that delivers any such types of therapy.

An exemplary standalone humidifier apparatus is described in WO 2015/038013. The contents of that specification are incorporated herein in their entirety by way of reference.

The standalone humidifier apparatus may have any one or more of the features described or shown herein.

Reference to any prior art in this specification is not, and should not be taken as, an acknowledgement or any form of suggestion that the prior art forms part of the common general knowledge in the field of endeavour in any country in the world.

Where reference is used herein to directional terms such as ‘up’, ‘down’, ‘forward’, ‘rearward’, ‘horizontal’, ‘vertical’ etc, those terms refer to when the apparatus is in a typical in-use position and/or with reference to particular orientations shown in the figures, and are used to show and/or describe relative directions or orientations.

1.-8. (canceled)

9. A portable display unit for a breathing assistance apparatus, the portable display unit comprising:
a display unit housing;
a screen; and
a cable configured for physically tethering the portable display unit with a base unit of the breathing assistance apparatus.

10. The portable display unit according to claim 9, wherein the cable is resiliently flexible and/or is retractable.

11. The portable display unit according to claim 9, wherein the cable is configured for communicating power to the portable display unit from the base unit and/or for communicating data between the portable display unit and the base unit.

12. The portable display unit according to claim 9, further comprising at least one wire for communicating power to the portable display unit from the base unit and/or for communicating data between the portable display unit and the base unit.

13. The portable display unit according to claim 9, further comprising a wireless communication interface for communicating data between the portable display unit and the base unit.

14. The portable display unit according to claim 9, further comprising at least one connection feature for removably connecting the portable display unit to the base unit.

15. A portable display unit for a breathing assistance apparatus having a base unit, the portable display unit comprising:
a display unit housing;
a screen;
a communication interface configured for communicating data between the base unit and the portable display unit; and
at least one connection feature configured for removably connecting the portable display unit to the base unit.

16. The portable display unit according to claim 15, wherein the at least one connection feature is arranged to removably connect the portable display unit to a housing of the base unit.

17. The portable display unit according to claim 16, wherein the at least one connection feature is arranged to removably connect the portable display unit to the housing of the base unit by a sliding action of the portable display unit relative to the base unit.

18. The portable display unit according to claim 15, further comprising at least one support feature for supporting the portable display unit on a support surface.

19. The portable display unit according to claim 18, wherein the at least one support feature and the at least one connection feature are provided by the same structural element.

20. The portable display unit according to claim 18, wherein the at least one support feature is arranged to support the portable display unit on a substantially horizontal support surface or on a medical pole.

21. The portable display unit according to claim 18, wherein the at least one support feature is arranged to enable an angle of the screen to be adjusted relative to the support surface.

22. The portable display unit according to claim 15, wherein an angular orientation of the screen is configured to be adjusted relative to the display unit housing or wherein the angular orientation of the screen is configured to be adjusted relative to the at least one connection feature.

23. The portable display unit according to claim 15, wherein the communication interface comprises at least one wire or wire connector for communicating the data and/or control signals between the portable display unit and the base unit.

24. The portable display unit according to claim 15, wherein the communication interface comprises a wireless communication interface for communicating the data and/or control signals between the portable display unit and the base unit.

25-108. (canceled)
